(19) United States
(12) Patent Application Publication
(10) Pub. No.: US 2022/0396801 A1
(43) Pub. Date: Dec. 15, 2022

(54) RIBOSOME TERMINATION STRUCTURES AND USE THEREOF

(71) Applicants: B. G. NEGEV TECHNOLOGIES AND APPLICATIONS LTD., AT BEN-GURION UNIVERSITY, Beer Sheva (IL); RAMOT AT TEL-AVIV UNIVERSITY LTD, Tel Aviv (IL)

(72) Inventors: Tamir TULLER, Tel Aviv (IL); Michael PEERI, Tel Aviv (IL); Yonatan CHEMLA, Beer Sheva (IL); Lital ALFONTA, Omer (IL)

(21) Appl. No.: 17/870,607

(22) Filed: Jul. 21, 2022

Related U.S. Application Data
(63) Continuation of application No. PCT/IL2021/050075, filed on Jan. 24, 2021.
(60) Provisional application No. 62/964,821, filed on Jan. 23, 2020.

(51) Int. Cl. C12N 15/67

(52) U.S. Cl. CPC C12N 15/67

(57) ABSTRACT

Nucleic acid molecule and vectors comprising regions of high or low folding energy are provided. Methods of producing coding sequences optimized for protein expression comprising introducing a mutation that increases or decreased folding energy are also provided.

### CROSS REFERENCE TO RELATED APPLICATIONS

This application is a National Phase of PCT Patent Application No. PCT/IL2021/050075 entitled “RIBOSOME TERMINATION STRUCTURES AND USE THEREOF”, having International filing date of Jan. 24, 2021, which claims the benefit of priority of U.S. Provisional Patent Application No. 62/964,821, filed Jan. 23, 2020 entitled “RIBOSOME TERMINATION SITES AND USE THEREOF”, the contents of which are all incorporated herein by reference in their entirety.

### FIELD OF INVENTION

The present invention is in the field of translational regulation.

### BACKGROUND OF THE INVENTION

To initiate protein translation, a ribosome binds and assembles an initiation complex in the area of the gene start codon. When monocistronic mRNA encoding a single gene is translated, spatial considerations that could interfere with ribosome binding are largely irrelevant. However, in bacteria, where a single mRNA transcript can contain several genes clustered into an operon, translation initiation must account for the space between genes. Specifically, how does translation initiation of a downstream operon gene occur without interference from the translating ribosome of the upstream gene? Despite a considerable understanding of protein translation in bacteria, this largely remains an unanswered question. Indeed, the mechanisms which control translation initiation in operons remain a matter of debate.

In bacterial operons, the intergenic distance between most of neighboring cistrons is shorter than 25-30 nucleotides. This distance is too small to simultaneously accommodate one ribosome terminating on the stop codon of the proximal gene and a second ribosome initiating de novo translation on the start codon of the distal gene. Translation re-initiation, a scenario whereby the terminating proximal ribosome does not dissociate from the mRNA after termination and instead re-initiates translation on the neighboring distal cistron, alleviates this problem. Presently, the mechanisms regulating translation re-initiation are not well understood. Specifically, regulators that determine whether a ribosome dissociates from or remains bound to the mRNA re-initiates translation have yet to be discovered.

Translation re-initiation affords bacteria the ability to translate operon-sequestered genes without significant interference between terminating and initiating ribosomes. However, translation re-initiation also carries risk. Uncontrolled, re-initiated translation could evoke high fitness costs due to ribosomes devoting more time to scanning than to translation or because of unintended translation re-initiation events. Indeed, as the ribosome can re-initiate in all possible frames and recognizes several start codons and alternative SD sequences (Tables 1 & 2), unintended translation re-initiation is of real concern, as demonstrated hereinbelow (FIG. 17A-D). As such, regulation of translation re-initiation is needed in nature and a better understanding of this phenomenon as well as molecules and methods of exploiting ribosome reinitiating are also needed for enhancing research as well as industry and medicine.

### SUMMARY OF THE INVENTION

The present invention provides nucleic acid molecules and vectors comprising regions of high or low folding energy. Methods of producing coding sequences optimized for protein expression comprising introducing a mutation that increases or decreased folding energy are also provided.

According to a first aspect, there is provided a nucleic acid molecule comprising:
a. at least two coding sequences, wherein a start codon of a second coding sequence is within 100 nucleotides of a stop codon of a first coding sequence; and
b. a region from 7 to 75 nucleotides downstream of the stop codon of the first coding sequence, wherein the region comprises:
   i. a fragment of a naturally occurring 3′ UTR comprising a mutation that increases folding energy of the region or of RNA encoded by the region;
   ii. at least a portion of the second coding sequence comprising at least one codon substituted to a different codon wherein the substitution increases folding energy of the region or of RNA encoded by the region; or
   iii. an artificial sequence configured such that a folding energy of the region or RNA encoded by the region is above a predetermined threshold.

According to some embodiments, the nucleic acid molecule is an RNA molecule, or wherein the nucleic acid molecule is a DNA molecule encoding a single RNA molecule comprising the at least two coding sequences.

According to some embodiments, the nucleic acid molecule of the invention is devoid of an internal ribosome entry site (IRES) between the at least two coding sequences.

According to some embodiments, the stop codon of the first coding sequence is upstream of a translational start site of the second coding sequence.

According to some embodiments, the region induces ribosome translational re-initiation at a start codon of the second coding sequence.

According to some embodiments, the region induces ribosome retention at the stop codon of the first coding sequence.

According to some embodiments, the start codon of the second coding sequence is within 50 nucleotides of the stop codon of the first coding sequence.

According to some embodiments, the region comprises a sequence selected from GCTGGX₁₂ (SEQ ID NO: 55) wherein X₁₂ is selected from C and T, ATTGAAX₁₃X₁₄ (SEQ ID NO: 56) wherein X₁₃ is A, T or C and X₁₄ is A or C, CTGX₁₅TGX₁₆ (SEQ ID NO: 57) wherein X₁₅ is A or C and X₁₆ is A, C or G, X₁₇GX₁₈X₁₉GCGX₂₀G (SEQ ID NO: 58) wherein X₁₇ is T or C, X₁₈ is T or C, X₁₉ is C or G, X₂₀ is T or C, X₂₁AX₂₂X₂₃AATX₂₄A (SEQ ID NO: 59) wherein X₂₁ is A or C, X₂₂ is A or G, X₂₃ is A or C, X₂₄ is A or G, TX₂₅GCCGC (SEQ ID NO: 60) wherein X₂₅ is C or T, X₂₆TGAAATX₂₇A (SEQ ID NO: 61) wherein X₂₆ is C or G and X₂₇ is G or A, GCCX₂₈GGC (SEQ ID NO: 62) wherein X₂₈ is T or G, TX₂₉TTTAX₃₀X₃₁G (SEQ ID NO: 63) wherein X₂₉ is T or C, X₃₀ is T or C, X₃₁ is T or C, and ATGX₃₂X₃₃TX₃₄AX₃₅ (SEQ ID NO: 64) wherein X₃₂ is A, G or T, X₃₃ is G, C or T, X₃₄ is G or A and X₃₅ is A or T.

According to some embodiments, the region comprises X₃₆GCTGGX₁₂X₃₇X₃₈ (SEQ ID NO: 65), wherein X₃₆ is C, T or G, X₁₂ is C or T, X₃₇ is G, C or A and X₃₈ is C, T, G or A.

According to another aspect, there is provided a nucleic acid molecule comprising:
a. a coding sequence comprising a stop codon; and
b. a region from 7 to 75 nucleotides downstream of the stop codon, wherein the region comprises:
   i. a fragment of a naturally occurring 3′ UTR comprising a mutation that decreases folding energy of the region or RNA encoded by the region; or
   ii. an artificial sequence configured such that a folding free energy of the region or RNA encoded by the region is below a predetermined threshold.

According to some embodiments, the region increases ribosome termination at the stop codon.

According to some embodiments, the region increases ribosome dissociation from the stop codon.

According to some embodiments, the nucleic acid molecule is an RNA molecule or a DNA molecule.

According to some embodiments, the region comprises a sequence selected from X₁X₂AAAX₃AA (SEQ ID NO: 45) wherein X₁ is selected from A and G, X₂ is selected from T and C and X₃ is selected from A and T, X₄GCGGCX₅ (SEQ ID NO: 46) wherein X₄ is G or C and X₅ is A or G, X₆X₇CGGGX₈AA (SEQ ID NO: 47) wherein X₆ is G or A, X₇ is C or G and X₈ is C or G, CTGATGACA (SEQ ID NO: 48), TGAAAAA (SEQ ID NO: 49), GGGX₉GAGGG (SEQ ID NO: 50) wherein X₉ is A, T, C or G, TGCCGGX₁₀ (SEQ ID NO: 51) wherein X₁₀ is G or A, CGCCAGC (SEQ ID NO: 52) and X₁₁CCGGCA (SEQ ID NO: 53) wherein X₁₁ is T or C.

According to some embodiments, the region comprises ATAAAAAA (SEQ ID NO: 54).

According to some embodiments, the region is from 7 to 40 nucleotides downstream of the stop codon.

According to some embodiments, the fragment is a fragment of a naturally occurring bacterial 3′ UTR.

According to some embodiments, the fragment is between 20-100 nucleotides in length.

According to some embodiments, the folding energy is local folding energy within a window of nucleotides.

According to some embodiments, the increase or decrease is an increase or decrease of at least 1 kcal/mol/40 bp.

According to some embodiments, the substitution is a synonymous substitution.

According to some embodiments, the predetermined threshold is −6 kcal/mol/40 bp.

According to some embodiments, the region is devoid of Rho-independent transcription terminators.

According to another aspect, there is provided an expression vector, comprising a nucleic acid molecule of the invention.

According to another aspect, there is provided an expression vector comprising:
a. a first region configured for insertion of a first coding sequence, or comprising a first coding sequence;
b. a second region configured for insertion of a second coding sequence, or comprising a second coding sequence, wherein a start of the second region is within 100 nucleotides from an end of the first region; and
c. a third region within 75 nucleotides downstream of the end of the first region, comprising:
   i. a fragment of a naturally occurring 3′ UTR comprising a mutation that increases folding energy of the third region or RNA encoded by the third region; or
   ii. an artificial sequence configured such that a folding energy of the third region or RNA encoded by the third region is above a predetermined threshold.

According to some embodiments, the vector is an RNA molecule, or wherein the vector is a DNA molecule encoding a single RNA molecule comprising the first coding sequence and the second coding sequence.

According to some embodiments, the vector of the invention is devoid of an internal ribosome entry site (IRES) between the at least two coding sequences.

According to some embodiments, the first region comprises a first coding sequence and a stop codon of the second region is within 100 nucleotides of the stop codon, or the second region comprises a second coding sequence and a translational start site (TSS) of the second coding sequence is within 100 nucleotides of the first region.

According to some embodiments, the third region induces ribosome translational re-initiation within the second region.

According to some embodiments, the third region induced ribosome retention at the stop codon.

According to some embodiments, the third region comprises a sequence selected from GCTGGX₁₂ (SEQ ID NO: 55) wherein X₁₂ is selected from C and T, ATTGAAX₁₃X₁₄ (SEQ ID NO: 56) wherein X₁₃ is A, T or C and X₁₄ is A or C, CTGX₁₅TGX₁₆ (SEQ ID NO: 57) wherein X₁₅ is A or C and X₁₆ is A, C or G, X₁₇GX₁₈X₁₉GCGX₂₀G (SEQ ID NO: 58) wherein X₁₇ is T or C, X₁₈ is T or C, X₁₉ is C or G, X₂₀ is T or C, X₂₁AX₂₂X₂₃AATX₂₄A (SEQ ID NO: 59) wherein X₂₁ is A or C, X₂₂ is A or G, X₂₃ is A or C, X₂₄ is A or G, TX₂₅GCCGC (SEQ ID NO: 60) wherein X₂₅ is C or T, X₂₆TGAAATX₂₇A (SEQ ID NO: 61) wherein X₂₆ is C or G and X₂₇ is G or A, GCCX₂₈GGC (SEQ ID NO: 62) wherein X₂₈ is T or G, TX₂₉TTTAX₃₀X₃₁G (SEQ ID NO: 63) wherein X₂₉ is T or C, X₃₀ is T or C, X₃₁ is T or C, and ATGX₃₂X₃₃TX₃₄AX₃₅ (SEQ ID NO: 64) wherein X₃₂ is A, G or T, X₃₃ is G, C or T, X₃₄ is G or A and X₃₅ is A or T.

According to some embodiments, the third region comprises X₃₆GCTGGX₁₂X₃₇X₃₈ (SEQ ID NO: 65), wherein X₃₆ is C, T or G, X₁₂ is C or T, X₃₇ is G, C or A and X₃₈ is C, T, G or A.

According to another aspect, there is provided an expression vector comprising:
a. a first region for insertion of a coding sequence; and
b. a second region within 100 nucleotides downstream of the first region comprising:
   i. a fragment of a naturally occurring 3′ UTR comprising a mutation that decreases folding energy of the second region or of RNA encoded by the second region; or
   ii. an artificial sequence configured such that a folding energy of the second region or RNA encoded by the second region is above a predetermined threshold.

According to some embodiments, the second region increases ribosome termination at a stop codon of the coding sequence.

According to some embodiments, the second region increases ribosome dissociation at a stop codon of the coding sequence.

According to some embodiments, the second region comprises a sequence selected from SEQ ID NO: 45-53.

According to some embodiments, the second region comprises SEQ ID NO: 54.

According to some embodiments, the vector is a DNA vector or an RNA vector.

According to some embodiments, the second region is devoid of Rho-independent transcription terminators.

According to some embodiments, the expression vector is a bacterial expression vector.

According to some embodiments, the region configured for insertion of a coding sequence is a multiple cloning site (MCS).

According to some embodiments, the fragment is a fragment of a naturally occurring bacterial 3′ UTR.

According to some embodiments, the fragment is between 20-100 nucleotides in length.

According to some embodiments, the increase or decrease is an increase or decrease of at least 1 kcal/mol/40 bp.

According to some embodiments, the predetermined threshold is −6 kcal/mol/40 bp.

According to another aspect, there is provided a method for producing a nucleic acid molecule optimized for expression of a second protein encoded by a second sequence comprising a translational start site (TSS) not more than 100 nucleotides away from a first stop codon of a first sequence encoding a first protein, the method comprising: introducing a mutation into a region from 7 to 75 nucleotides downstream of the first stop codon; wherein the mutation increases folding energy of the region or of RNA encoded by the region.

According to some embodiments, the nucleic acid molecule is an RNA molecule, or wherein the nucleic acid molecule is a DNA molecule encoding a single RNA molecule comprising the first sequence encoding the first protein and the second sequence encoding the second protein.

According to some embodiments, the nucleic acid molecule is devoid of an internal ribosome entry site (IRES) between the first sequence encoding the first protein and the second sequence encoding the second protein.

According to some embodiments, the first stop codon is upstream of the TSS of the sequence encoding the second protein.

According to some embodiments, the method of the invention is for producing a nucleic acid molecule with increased ribosome translational re-initiation at the TSS of the second sequence encoding the second protein.

According to some embodiments, the mutation is within a sequence selected from SEQ ID NO: 44-53, and wherein said mutation produces a sequence that does not comprise any of SEQ ID NO: 44-53.

According to another aspect, there is provided a method for producing a nucleic acid molecule optimized for expressing a first protein comprising a stop codon, the method comprising: introducing a mutation into a region from 7 to 75 nucleotides downstream of the stop codon; wherein the mutation decreases folding energy of the region or of an RNA encoded by the region.

According to some embodiments, the method of the invention is for producing a nucleic acid molecule with increased ribosome termination at the stop codon of a coding sequence.

According to some embodiments, the method of the invention is for producing a nucleic acid molecule with increased ribosome dissociation at a stop codon of the coding sequence.

According to some embodiments, the nucleic acid molecule is a DNA molecule or an RNA molecule.

According to some embodiments, the mutation is within a sequence selected from SEQ ID NO: 55-64 and wherein said mutation produces a sequence that does not comprise any of SEQ ID NO: 55-64.

According to some embodiments, the optimizing is optimizing expression in a bacterial cell.

According to some embodiments, the method comprises introducing a mutation into a region from 7 to 40 nucleotides downstream of the stop codon.

According to some embodiments, the nucleic acid molecule further comprises at least one regulatory region operatively linked to a first coding sequence encoding the first protein, wherein the at least one regulatory region is sufficient to drive expression of the first coding sequence.

According to some embodiments, the nucleic acid molecule is genomic DNA and the introducing a mutation comprises genome editing.

According to another aspect, there is provided a method of converting an overlapping gene pair into two non-overlapping genes, the method comprising:
a. receiving a sequence of the overlapping gene pair comprising a first coding sequences of a first gene of the gene pair and a second coding sequence of a second gene of the gene pair, wherein a start codon of the second coding sequence is within the first coding sequence;
b. inserting the second coding sequence not more than 100 nucleotides downstream of a stop codon of the first coding sequence;
c. producing between 7 to 75 nucleotides downstream of the stop codon of the first coding sequence a region, wherein the region or RNA encoded by the region comprises high folding energy;
thereby converting an overlapping gene pair into two non-overlapping genes.

According to some embodiments, the sequence is a DNA sequence or an RNA sequence.

According to some embodiments, the sequence is a DNA sequence selected from a vector sequence and a genomic sequence.

According to some embodiments, the inserting the second coding sequence comprises deleting a 3′ portion of the second coding sequence that was not overlapping with the first coding sequence.

According to some embodiments, the inserting is not more than 40 nucleotides downstream of the stop codon of the first coding sequence.

According to some embodiments, the producing comprises generating a mutation that increases folding energy of the region.

According to some embodiments, the mutation is within the inserted second coding region and the mutation is a synonymous mutation.

According to some embodiments, the mutation produces a sequence selected from SEQ ID NO: 44-53.

According to some embodiments, the producing comprises inserting a region of high folding energy.

According to some embodiments, high folding energy is folding energy above a predetermined threshold.

According to some embodiments, high folding energy is above −6 kcal/mol/40 bp.

According to another aspect, there is provided a computer program product comprising a non-transitory computer-readable storage medium having program instructions embodied therewith, the program instructions executable by at least one hardware processor configured to:
a. receive a sequence of a nucleic acid molecule comprising at least two coding sequences, wherein a start codon of a second coding sequence is proximal to a stop codon of a first coding sequence;
b. determine within a region around a stop codon of the first coding sequence at least one mutation that increases folding energy of the first region or RNA encoded by the first region; and
c. output
   i. a mutated sequence of the nucleic acid molecule comprising the at least one mutation, or
   ii. a list of possible mutations in the region that increase folding energy of the region or RNA encoded by the region.

According to another aspect, there is provided a computer program product comprising a non-transitory computer-readable storage medium having program instructions embodied therewith, the program instructions executable by at least one hardware processor configured to:
a. receive a nucleic acid molecule comprising a coding sequence;
b. determine within a region around a stop codon of the coding sequence at least one mutation that decreases folding energy of the region or RNA encoded by the region; and
c. output
   i. a mutated sequence of the nucleic acid molecule comprising the at least one mutation, or
   ii. a list of possible mutations in the region that decrease folding energy of the region or RNA encoded by the region.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-H: mRNA secondary structure (ΔG_{fold}) controls distal operon gene expression. (1A) Synthetic operon design and FACS-sorting scheme. (1B) Histograms of GFP and RFP fluorescence of 10⁵ clones. (1C) Dot plot sorting of 10⁶ cells into color-coded bins with constant RFP levels and variable GFP levels (top); Histograms of GFP distribution in 3,000 cells from each bin after sorting (bottom). (1D-F) (1D) Correlation between the population mean GFP expression levels and the weighted mean of ΔG_{fold} of 3×10³ unique sequences in each bin. The x and y axes error bars represent the 99% confidence interval and relative standard deviation, respectively. Spearman correlation was performed on the weighted averages of the six bins (n=6, p=1, p-value=0.0028). Correlation between GFP expression and ΔG_{fold} of (1E) all (n=33) isolated variants, and (1F) a subset (n=8) presenting an AUG start codon at position +3 or +4. (1G) mRNA secondary structure and ΔG_{fold} landscape of variable sequences of two distinct clones (111, 207). (1H) Schematic representation of the role of the RTS in distal operon gene translation (ribosomes are not drawn to scale).

FIGS. 2A-F: RTSs are conserved across bacterial phyla. (2A) Pipeline for genome-wide RTS analysis. ΔLFE analysis reveals that, on average, RTS is present and localized downstream of stop codons across (2B) E. coli (orange) (2C) B. subtilis (green) and 128 bacterial species examined (blue). The RTS signal is more significant in genes encoding highly abundant products in (2D) E. coli, and (2E) all bacterial species for which protein abundance data is available. (2F) ΔLFE heatmap depicting the 100 nucleotide-long region around stop codons across bacteria (warm colors: stronger folding than expected; cool colors: weaker folding than expected). The purple bar, left of each species heatmap, represents the fraction of genes in which RTS was found under the RTS statistical model described in the Material and Methods section.

FIGS. 3A-K: RTS is a translation re-initiation regulator. (3A) ΔLFE standard deviation landscape around the stop codon. (3B) E. coli gene density plot (Z-axis) versus ΔLFE (X-axis) and distance from a stop codon (Y-axis). Different colors are used for improved visualization. Inset shows gene density at position zero. Grey represents the intersection of the two groups. The RTS profile around the stop codon depends on the inter-cistronic distance before the downstream gene in (3C) E. coli and (3D) 128 bacterial species. All parameters used to calculate ΔLFE are constant across all figures and relied on a window size of 40 nucleotides. (3E) Representative anti-His-tag Western blot (top) and the mean of n=3 fluorescence measurements (error bars represent standard error; bottom) of eight AUG (+3/4) clones, with ΔG_{fold} indicated. (3F) Mass spectrometry analysis of GFP from selected library clones, with the codon and location used for re-initiation indicated. Representative cropped Western blots of seven random E. coli clones (3G) without or (3H) with stop codon reassignment, each in the presence (left) or absence (right) of RF1. (3I) Genetic constructs of operonic and monocistronic GFP. Each anti-His-tag Western blot represents a comparison, normalized to OD, between the two constructs for each of six tested clones. (3J) The mean fluorescence measurements comparing the two constructs. Error bars represent standard deviation. Significance was determined by Welch two-sample t-tests (from left to right; df=22.0, p=0.4164; df=4.5, p=0.1091; df=6.3, p-value=0.0854; df=20.9, p-value=0.0397; df=16.3, p-value=0.00061; df=4.3, p-value=0.0067). (3K) Spearman correlation (n=6, p=0.94, p-value=0.017), between the ratio of operonic to monocistronic GFP levels and ΔG_{fold} of each clone. Uncropped Western blots are available (FIG. 12A-E). Ribosomes are not drawn to scale.

FIGS. 4A-B: In all bacteria phyla, RTSs are enriched where re-initiation is deleterious and depleted where re-initiation is advantageous. (4A) RTS presence depends on operonic position in E. coli and in all operon-mapped bacterial species. The blue curves represent the average ΔLFE of first and middle operon genes, while the red curve represents terminal operon genes. (4B) RTS presence depends on downstream cistron directionality in 128 bacterial species.

FIGS. 5A-G: Flow Cytometry gating and negative control. (5A) A negative control, which consists of WT E. coli MG1655. (5B) First size gating. (5C) Second size gating. (5D) Uncropped sorting with gate and population statistics. (5E) The weighted mean of ΔG_{fold} with 99% confidence intervals of N=˜3×10³ unique sequences in each bin. Significance levels were determined by two-sided Wilcoxon test and all tested conditions were found significant. Error bars represent the 99% confidence intervals. (5F) Sorting by GFP fluorescence of the eight-clone subgroup where one of the three most abundant start codons are present in position+3 or +4 from the RFP stop codon. An increase in GFP levels in each clone population negatively correlates with the increase in the negative value of ΔG_{fold} of the intergenic region between the RFP and GFP genes. (5G) Simulated RNA folding of large representative samples (n=10⁶) from the sequence-space under constraints that were imposed on the random library (24 random followed by 13 fixed nucleotides; 24+13nt; red), under the constraints but with all 37 nucleotides randomized (37nt; blue), and unconstrained (green). The folding energies of all sample populations are gamma-distributed as expected, sample statistics are summarized in the figure table; all units are kcal mol⁻¹ window⁻¹. The statistical values are in agreement with experimental results, which show that all populations clustered around the constrained means, as detailed in the manuscript. If one considers that the FACS sorting and GFP expression of individual bacteria are both noisy, this simulation could well explain the central tendency of population distribution we observed in our study.

FIG. 6: Quantitative PCR of synthetic operon mRNA levels. mRNA abundance fold change (left) measured by two experimental repeats of qPCR, each with two or three replications of twelve select clones, including the eight clones from the subgroup described in FIG. 1F. Fold change is relative to the average mRNA abundance of all clones. No significant correlation was noted between ΔG_{fold} of the variable region in several pRNXG clones and mRNA abundance in E. coli MG1655 (scatter plots; right), error bars represent a standard deviation of the mean. This was confirmed with amplicons of regions up-stream (RFP amplicon) and down-stream (GFP amplicon) of the variable sequence region. All amplicons were normalized to 16S rRNA amplicon abundance, and the primer efficiencies were >99%. The no-template controls (NTC) quantitation cycles (CQ) were at least 15 cycles larger than samples.

FIGS. 7A-C: RFP expression from different synthetic operon clones. (7A) Mean expression levels of RFP normalized to OD₆₀₀ measured by RFP fluorescence; error bars represent standard error of experimental repeats, the number of experimental repeats for each clone is represented by the number of points scattered, but for all clones, at least three measurements were taken (n≥3). (7B) Correlation between RFP fluorescence levels and ΔG_{fold}. No significant correlation was observed (Spearman correlation=−0.19, S=7,118, n=33, p-value=0.29). (7C) Dependence between GFP and RFP expression levels of the synthetic operon. No significant correlation was observed (Spearman correlation=0.08, S=5,528 p-value=0.67).

FIGS. 8A-D: Bacterial growth rates of isolated library clones. (8A) Representative bacterial growth curves, presenting the average OD₆₀₀ over time of n=3 technical replicates, for all clones used in this study. (8B) The average maximal OD₆₀₀ achieved by each clone, error bars represent the standard error of each clone. (8C) The left panel presents the linear Fischer correlation between RFP levels, and bacterial growth was found to be significant regardless of the clone-specific genotype (n=33, F=39.11, adjusted r²=0.54, P-value=5.978e⁻⁰⁷). The right panel presents the linear Fischer correlation between GFP levels, and bacterial growth was found to be non-significant. This can be interpreted as the effect of each clone-specific genotype on GFP expression is more substantial than the contribution of bacterial density (n=33, F=0.7106, adjusted r²=−0.001, P-value=0.41). (8D) The linear Fischer correlation between bacterial growth and ΔG_{fold} of the variable sequence of each clone was found to be non-significant (n=33, F=0.04, adjusted r²=−0.03, P-value=0.8466).

FIGS. 9A-B: (9A) RTS presence across all kingdoms of life (all stop codons aggregated). Parameter sensitivity and effect of different ΔG thresholds on the number of RTS containing genes, under the RTS model (see methods), for all bacteria (N=128). The selected threshold value of −6.0 kcal mol⁻¹ window⁻¹, for the heat maps presented in FIG. 2F, and FIG. 9B is highlighted. (9B) ΔLFE landscape of in 128 bacteria, 59 archaea, and 8 eukaryotes. The ΔLFE landscape was depicted as a heatmap of 100 nucleotide-long regions around stop codons in species belonging to domains comprising the three branches of the tree of life (warm colors: stronger folding than expected; cool colors: weaker folding than expected). Using the RTS model (see Materials and Methods), we assessed the presence or absence of the RTS. The results revealed that 122/128 (95.3%) of bacteria, 12/49 (24.5%) of archaea and 2/8 (25.0%) of eukaryotes present an apparent RTS, although the sample sizes of the two latter groups are too small and the RTS signal is too weak and unreliable to draw any conclusions at this time.

FIGS. 10A-C: Densitometric analysis of Western blots (10A) Anti-His tag Western blot of random clones. For the randomly selected clones (red) and for the clones with an AUG start codon beginning at positions +3 or +4 (cyan), both (10B) the 55 kDa RFP-GFP product resulting from stop codon read-through, and (10C) the 28 kDa GFP product resulting from de novo initiation or re-initiation were measured using densitometry of the pRXNG clones in E. coli MG1655. The results were aggregated experimental repeats of each clone as a box-plot (top) and as scatterplots for correlation analyses (bottom). In the scatterplots, each data point represents one experimental anti-His tag Western blot repeat of a clone with the indicated calculated ΔG_{fold}. The 28 kDa GFP product accounts for 91% of the correlation between ΔG_{fold} and the total amount of GFP expressed by the different clones (omega squared test, ω²=0.91). Moreover, correlation with ΔG_{fold} was maintained for GFP (Spearman correlation ρ=0.80, n=58, S=6479, p-value=4.537e-14) and also, albeit to a lesser degree, with the 55 kDa read-through product (Spearman correlation ρ=0.50, n=58, S=16326, p-value=7.011e-5).

FIG. 11: Mass spectra of different clones. Five clones expressing sufficient levels of the ˜28 kDa GFP product and a representative read-through product (with the UAG stop codon mutated to encode tyrosine) were purified using nickel affinity columns and subjected to mass spectrometry to identify the start codon. These involved comparisons of calculated masses generated by the clone-specific sequence and the measured mass of the protein. Left panels depict the raw MS results, while the right panels depict de-convoluted data obtained using Promass software. In the manuscript, we report the primary product of each clone. However, we cannot exclude or accurately assess the possibility of multiple possible initiation sites with different efficiencies.

FIGS. 12A-E: Correlation between ΔG_{fold} and GFP levels without and with Release Factor 1 (RF1) (12A) Comparison of GFP expression, measured by fluorescence, between E. coli C321.ΔprfA EXP and MG1655, both transformed with the pEVOL pylRS genetic code expansion system and five pRXNG library clones with different ΔG_{fold}. Each data point represents the average of n=3 experimental replicates. (12B) Uncropped anti-His-tag Western blots presented in FIG. 3E of eight pRXNG clones with AUG start codon in the 3^{rd} of 4^{th} codon downstream from the RFP stop codon. This experiment was repeated independently with similar results 4 times (12C) Uncropped anti-His-tag Western blots presented in FIG. 3G of five pRXNG library clones with different ΔG_{fold}. This experiment was repeated independently with similar results 4 times. (12D) Uncropped gels presented in FIG. 3H. The bands below the RFP-GFP product (with a size of ˜50 kDa) are the his-tagged pyrrolysyl synthetase (pylRS) gene from the co-transformed pEVOL plasmid which is used for genetic code expansion is transformed. This experiment was repeated independently with similar results four times. (12E) The uncropped blot of FIG. 3I. This experiment was repeated independently once.

FIG. 13: Analysis of operonic position effect on RTS presence with/without a down-stream AUG start codon Left panel: Terminal operonic genes either with or without an AUG start codon in-frame of the down-stream CDS in the 50 nucleotides (nt) downstream of a stop codon. Right panel: Mid-operonic genes either with or without an AUG start codon in-frame of the down-stream CDS in the 50 nt downstream of a stop codon. We examined differences between two groups of genes, namely those assuming the last position in an operon (i.e., terminal genes) (left) versus all other operon genes (i.e., non-terminal genes) (right). Each group was further divided according to the presence of an in-frame AUG start codon within 50 nt downstream of the stop codon or the absence of a start codon. Such divisions revealed that in terminal genes, where translation insulation is expected in all cases, significant selection for an RTS was observed, regardless of the presence or absence of a down-stream start codon. Conversely, in mid-operon genes, selection for RTSs in the group with the start codon, where re-initiation is expected, is not higher than random. In the second group, where re-initiation is not desired as no in-frame AUG start codon exists, significant selection for RTSs was observed.

FIG. 14: Genomic traits explain some of the variability in selection strength for RTS between species. Correlation between three genomic traits and ΔLFE (i.e., the strength of selection for the RTS) across 128 bacterial strains. Each dot represents one bacterial strain. r values and statistical significance are calculated using the Pearson correlation (n=128).

FIG. 15: Controlling for an RTS link to transcription termination Left panel: Analysis of E. coli genes grouped by transcription termination mechanism shows that folding bias cannot be explained by the presence of rho-independent terminators. Red, genes with rho-independent terminators. Blue, genes that are last in their transcription units (TU) but do not have rho-independent terminators. Green, all other genes. Lines represent ΔLFE, computed as described in the Methods section. Annotation of rho-independent genes based on WebGesTer-DB. Annotation of TU positions based on the ODB4 database. Right panel: The RTS signal shows no change between groups of genes with short (<50 nt) or long (>50 nt) 3′ UTRs.

FIG. 16: Dot plot of the correlation between observed GFP levels and those predicted upon de novo initiation using the RBS calculator.

FIGS. 17A-D: Probability of having a start codons downstream of a stop codon without selection (17A) The probability of having at least one efficient start codon (ATG, GTG, TTG, CTG, ATA, ATT) by chance as a function of DNA length. (17B) The probability that a sequence with no efficient start codon will generate an efficient start codon after a one nucleotide mutation as a function of strand length (Juke and Cantor, one parameter mutation model). (17C) The probability of having at least one efficient start codon through consecutive mutations on a fixed, 50 base pair-long DNA stretch. (17D) Density plot of mapped E. coli 3′ UTR lengths in the RegulonDB database (470 transcriptions units).

FIG. 18: Tables of top ten putative RTS and non-RTS motifs found in E. coli. Analyses of sequences motifs in RTS regions of E. coli. Logo plots of sequence motifs detected in the RTS regions across the E. coli genome significantly enriched sequences are only 1-2 in each column. E-value represents the probability of this motif to appear by chance, and Sites represent the number of genes that harbor this motif in the expected RTS region.

### DETAILED DESCRIPTION OF THE INVENTION

The present invention, in some embodiments, provides nucleic acid molecules and vectors comprising regions of high or low folding energy. The present invention further concerns methods of producing coding sequences optimized for protein expression.

The present invention is based on the following surprising findings. Here, a stable mRNA secondary structure was identified downstream of the stop codon (termed the RTS) that controls translation re-initiation. It was revealed that robust signals corresponding to the presence of an RTS are found across the E. coli genome. It was also showed that the RTS is conserved across bacterial phyla, with an RTS signal peaking at a position that correlates with the edge of the mRNA stretch that is shielded by a terminating ribosome, alluding to a RTS-ribosome interaction. The functional analyses and experiments performed here all support the RTS acting as a translational insulator, inhibiting translation re-initiation.

Currently, two competing models explain re-initiation, namely the classic 30S-binding model, where ribosomes dissociate from polycistronic mRNA upon gene translation termination, only to immediately re-bind, like de novo initiation, and translate the downstream cistron. In this mode, the expectation will be to detect the translation of a distal cistron by both re-initiating and de novo initiating ribosomes, which will compete over the RBS. The second, which was recently demonstrated, is the 70S-scanning model, where the ribosome does not dissociate but instead scans the downstream mRNA for a re-initiation site. The results provide herein support the latter model as de novo initiation was not observed, and the observed existence of an RTS in terminal genes is more parsimonious when scanning-based re-initiation occurs.

By a first aspect, there is provided a nucleic acid molecule comprising:
a. at least two coding sequences, wherein a start codon of a second coding sequence is proximal to a stop codon of a first coding sequence; and
b. a region around the stop codon of the first coding sequence, wherein the region or RNA encoded by the region comprises high and/or increased folding energy.

By another aspect, there is provided an expression vector comprising:
a. a first region configured for insertion of a first coding sequence, or comprising a first coding sequence;
b. a second region configured for insertion of a second coding sequence, or comprising a second coding sequence, wherein a start of the second region is proximal to and end of the first region; and
c. a third region around the end of the second region, wherein the third region or RNA encoded by the third region comprises high and/or increased folding energy.

By another aspect, there is provided a nucleic acid molecule comprising:
a. at least two coding sequences, wherein a start codon of a second coding sequence is proximal to a stop codon of a first coding sequence; and
b. a region around the stop codon of the first coding sequence, wherein the region or RNA encoded by the region comprises low and/or decreased folding energy.

By another aspect, there is provided an expression vector comprising:
a. a first region configured for insertion of a first coding sequence, or comprising a first coding sequence;
b. a second region configured for insertion of a second coding sequence, or comprising a second coding sequence, wherein a start of the second region is proximal to and end of the first region; and
c. a third region around the end of the second region, wherein the third region or RNA encoded by the third region comprises low and/or decreased folding energy.

In some embodiments, the nucleic acid molecule is selected from DNA and RNA. In some embodiments, the nucleic acid molecule is RNA. In some embodiments, the nucleic acid molecule is DNA. In some embodiments, the DNA molecule encodes a single RNA molecule comprising both of the at least two coding sequences. It will be understood by a skilled artisan that the invention relates to RNA or production of RNA with at least two coding regions wherein after translational termination of the first sequence there is ribosome re-initiation at the start codon of the second sequence. Thus, either the molecule must be a single polycistronic RNA or a DNA that encodes a polycistronic RNA. In some embodiments, the region induces ribosome translational re-initiation at a start codon of the second coding sequence. In some embodiments, third region induces ribosome translational re-initiation within the second region. In some embodiments, the region induces ribosome retention at the stop codon. In some embodiments, ribosome retention at the stop codon comprises retention beyond the stop codon. In some embodiments, the region induces ribosome retention beyond the stop codon.

In some embodiments, the DNA is genomic DNA. In some embodiments, the DNA is vector DNA. In some embodiments, the DNA is cDNA. In some embodiments, the nucleic acid molecule is a vector. In some embodiments, the vector is an expression vector. In some embodiments, the expression vector is a prokaryotic expression vector. In some embodiments, the expression vector is a eukaryotic expression vector. In some embodiments, the vector is a bacterial expression vector. In some embodiments, the nucleic acid molecule is a heterologous transgene. In some embodiments, the nucleic acid molecule encodes a heterologous transgene.

In some embodiments, the nucleic acid molecule comprises at least two coding regions. In some embodiments, the nucleic acid molecule comprises at least two coding sequences. In some embodiments, the vector comprises at least two regions configured for insertion of a coding sequence. In some embodiments, at least two is a plurality. In some embodiments, at least two is at least two, at least 3, at least 4, at least 5, at least 6, at least 7, at least 8, at least 9 or at least 10. Each possibility represents a separate embodiment of the invention. In some embodiments at least two is two, three, four, five, six, seven, eight, nine or 10 coding sequences. Each possibility represents a separate embodiment of the invention. In some embodiments, at least two is two. In some embodiments, the coding sequence comprises a start codon. In some embodiments, the nucleic acid molecule comprises a stop codon. In some embodiments, the coding sequence comprises a stop codon. In some embodiments, a start codon is a translational start site. In some embodiments, a stop codon is the translational end site or the translational termination site. It will be understood by a skilled artisan that both DNA and RNA can be considered to have codons. Within a DNA molecule a codon refers to the 3 bases that will be transcribed into RNA bases that will act as a codon for recognition by a ribosome and will thus translate an amino acid. In some embodiments, the nucleic acid molecule further comprises an untranslated region (UTR). In some embodiments, the UTR is a 5′ UTR. In some embodiments, the UTR is a 3′ UTR.

As used herein, the term “coding sequence” refers to a nucleic acid sequence that when translated results in an expressed protein. In some embodiments, the coding sequence is to be used as a basis for making codon alterations. In some embodiments, the coding sequence is a gene. In some embodiments, the coding sequence is a viral gene. In some embodiments, the coding sequence is a prokaryotic gene. In some embodiments, the coding sequence is a bacterial gene. In some embodiments, the coding sequence is a eukaryotic gene. In some embodiments, the coding sequence is a mammalian gene. In some embodiments, the coding sequence is a human gene. In some embodiments, the coding sequence is a portion of one of the above listed genes. In some embodiments, the coding sequence is a heterologous transgene. In some embodiments, the above listed genes are wild type, endogenously expressed genes. In some embodiments, the above listed genes have been genetically modified or in some way altered from their endogenous formulation. These alterations may be changes to the coding region such that the protein the gene codes for is altered.

The term “heterologous transgene” as used herein refers to a gene that originated in one species and is being expressed in another. In some embodiments, the transgene is a part of a gene originating in another organism. In some embodiments, the heterologous transgene is a gene to be overexpressed. In some embodiments, expression of the heterologous transgene in a wild-type cell reduces global translation in the wild-type cell.

In some embodiments, the nucleic acid molecule or the expression vector further comprises a regulatory element. In some embodiments, regulatory element is configured to induce transcription of the coding sequence. In some embodiments, the regulatory element is a promoter. In some embodiments, the regulatory element is selected from an activator, a repressor, an enhancer, and an insulator. In some embodiments, the coding region is operably linked to the regulatory element. The term “operably linked” is intended to mean that the coding sequence is linked to the regulatory element or elements in a manner that allows for expression of a coding sequence (e.g., in an in vitro transcription/translation system or in a host cell when the vector is introduced into the host cell). In some embodiments, the promoter is a promoter specific to the expression vector. In some embodiments, the promoter is a viral promoter. In some embodiments, the promoter is a bacterial promoter. In some embodiments, the promoter is a eukaryotic promoter. In some embodiments, the promoter is an archaeal promoter.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, proximal is within 100 nucleotides. In some embodiments, proximal is within 75 nucleotides. In some embodiments, proximal is within 50 nucleotides. In some embodiments, the stop codon of the first coding sequence is upstream of the start codon of the second coding sequence. In some embodiments, the stop codon of the first coding sequence is downstream of the start codon of the second coding sequence. In some embodiments, proximal to a codon is proximal to the first base of the codon. In some embodiments, proximal to a codon is proximal to the last base of the codon.

In some embodiments, the region around the stop codon of the first coding sequence is downstream of the stop codon. In some embodiments, the region around the end of the first region is downstream of the first region. In some embodiments, the region around the end of the first region is upstream of the second region. In some embodiments, the region around the stop codon of the first coding sequence is the third region. In some embodiments, downstream is 3′ to. In some embodiments, upstream is 5′ to. In some embodiments, the end of the first coding sequence is a stop codon of the first coding sequence. In some embodiments, the end of the first coding sequence is beyond the end of a stop codon of the first coding sequence. In some embodiments, the end of the first coding sequence is a stop codon and beyond the stop codon of the first coding sequence. In some embodiments, beyond is just beyond. In some embodiments, just beyond is within 3, 5, 6, 9, 12, 15, 18, 20, 21, 24, 25, 27, 30, 33, 35, 36, 39, 40, 42, 45, 48, 50, 51, 54, 55, 57, 60, 63, 65, 66, 69, 70, 72, 75, 78, 80, 81, 84, 85, 87, 90, 93, 95, 96, 99 and 100 nucleotides. Each possibility represents a separate embodiment of the invention. In some embodiments, just beyond is within 100 nucleotides. In some embodiments, just beyond is within 70 nucleotides. In some embodiments, just beyond is within 50 nucleotides. In some embodiments, just beyond is within 40 nucleotides.

It will be understood that hereinbelow reference to “the region” refers either to embodiments in which there is only one region or to “the third region” in reference to embodiment with more than one region recited and wherein the region has increased/high folding energy or to “the second region” in reference to embodiments with more than one region recited and wherein the region has decreased/low folding energy. In some embodiments, the region is from the stop codon to 25, 30, 40, 50, 60, 70, 75, 80, 90, or 100 nucleotides downstream of the stop codon. Each possibility represents a separate embodiment of the invention. In some embodiments, the region is from the stop codon to 100 nucleotides downstream of the stop codon. In some embodiments, the region is from the stop codon to 75 nucleotides downstream of the stop codon. In some embodiments, the region is from the stop codon to 50 nucleotides downstream of the stop codon. In some embodiments, the region is from the stop codon to 40 nucleotides downstream of the stop codon. In some embodiments, the region includes the stop codon. In some embodiments, the region excludes the stop codon. It will be understood that for the purposes of numbering the third base of the stop codon will be considered base zero and so the first base after the stop codon will be considered base +1 relative to the stop codon, or base 1 downstream of the stop codon. In some embodiments, the region is from 1 to 25, 1 to 30, 1 to 40, 1 to 50, 1 to 60, 1 to 70, 1 to 75, 1 to 80, 1 to 90, or 1 to 100 nucleotides downstream of the stop codon. Each possibility represents a separate embodiment of the invention. In some embodiments, the region is from 1 to 100 nucleotides downstream of the stop codon. In some embodiments, the region is from 1 to 75 nucleotides downstream of the stop codon. In some embodiments, the region is from 1 to 50 nucleotides downstream of the stop codon. In some embodiments, the region is from 1 to 40 nucleotides downstream of the stop codon.

In some embodiments, the codons covered by the ribosome while it is reading the stop codon are not part of the region. In some embodiments, the region begins at 7 nucleotides downstream of the stop codon. It will be known by a skilled artisan that while the ribosome is reading the stop codon it will also be covering the next two codons, which is the next six nucleotides. As these nucleotides will be covered, they will not be free to interact with the region and will not be able to form secondary structure. In some embodiments, the region is from 7 to 100, 7 to 90, 7 to 80, 7 to 75, 7 to 70, 7 to 60, 7 to 50, 7 to 40, 7 to 30 or 7 to 25 nucleotides downstream of the stop codon. Each possibility represents a separate embodiment of the invention. In some embodiments, the region is from 7 to 100 nucleotides downstream of the stop codon. In some embodiments, the region is from 7 to 75 nucleotides downstream of the stop codon. In some embodiments, the region is from 7 to 50 nucleotides downstream of the stop codon. In some embodiments, the region is from 7 to 40 nucleotides downstream of the stop codon. In some embodiments, the region is from 9 to 100, 9 to 90, 9 to 80, 9 to 75, 9 to 70, 9 to 60, 9 to 50, 9 to 40, 9 to 30 or 9 to 25 nucleotides downstream of the stop codon. Each possibility represents a separate embodiment of the invention. In some embodiments, the region is from 9 to 100 nucleotides downstream of the stop codon. In some embodiments, the region is from 9 to 75 nucleotides downstream of the stop codon. In some embodiments, the region is from 9 to 50 nucleotides downstream of the stop codon. In some embodiments, the region is from 9 to 40 nucleotides downstream of the stop codon. In some embodiments, the region is from 5 to 100, 5 to 90, 5 to 80, 5 to 75, 5 to 70, 5 to 60, 5 to 50, 5 to 40, 5 to 30 or 5 to 25 nucleotides downstream of the stop codon. Each possibility represents a separate embodiment of the invention. In some embodiments, the region is from 5 to 100 nucleotides downstream of the stop codon. In some embodiments, the region is from 5 to 75 nucleotides downstream of the stop codon. In some embodiments, the region is from 5 to 50 nucleotides downstream of the stop codon. In some embodiments, the region is from 5 to 40 nucleotides downstream of the stop codon.

In some embodiments, the region comprises at least one of:
i. a fragment of a naturally occurring sequence 3′ to a stop codon comprising a mutation that increases folding energy of the region or of RNA encoded by the region;
ii. at least a portion of the second coding region comprising at least one codon substituted to a different codon, wherein the substitution increases folding energy of the region or of RNA encoded by the region; or
iii. an artificial sequence configured such that a folding energy of the region or of RNA encoded by the region is above a predetermined threshold.

In some embodiments, the region comprises a fragment of a naturally occurring sequence 3′ to a stop codon comprising a mutation that increases folding energy of the region or of RNA encoded by the region. In some embodiments, the region comprises at least a portion of the second coding region comprising at least one codon substituted to a different codon, wherein the substitution increases folding energy of the region or of RNA encoded by the region. In some embodiments, the region comprises an artificial sequence configured such that a folding energy of the region or of RNA encoded by the region is above a predetermined threshold.

In some embodiments, the region comprises at least one of:
i. a fragment of a naturally occurring sequence 3′ to a stop codon comprising a mutation that decreases folding energy of the region or of RNA encoded by the region;
ii. at least a portion of the second coding region comprising at least one codon substituted to a different codon, wherein the substitution decreases folding energy of the region or of RNA encoded by the region; or
iii. an artificial sequence configured such that a folding energy of the region or of RNA encoded by the region is below a predetermined threshold.

In some embodiments, the region comprises a fragment of a naturally occurring sequence 3′ to a stop codon comprising a mutation that decreases folding energy of the region or of RNA encoded by the region. In some embodiments, the region comprises at least a portion of the second coding region comprising at least one codon substituted to a different codon, wherein the substitution decreases folding energy of the region or of RNA encoded by the region. In some embodiments, the region comprises an artificial sequence configured such that a folding energy of the region or of RNA encoded by the region is below a predetermined threshold.

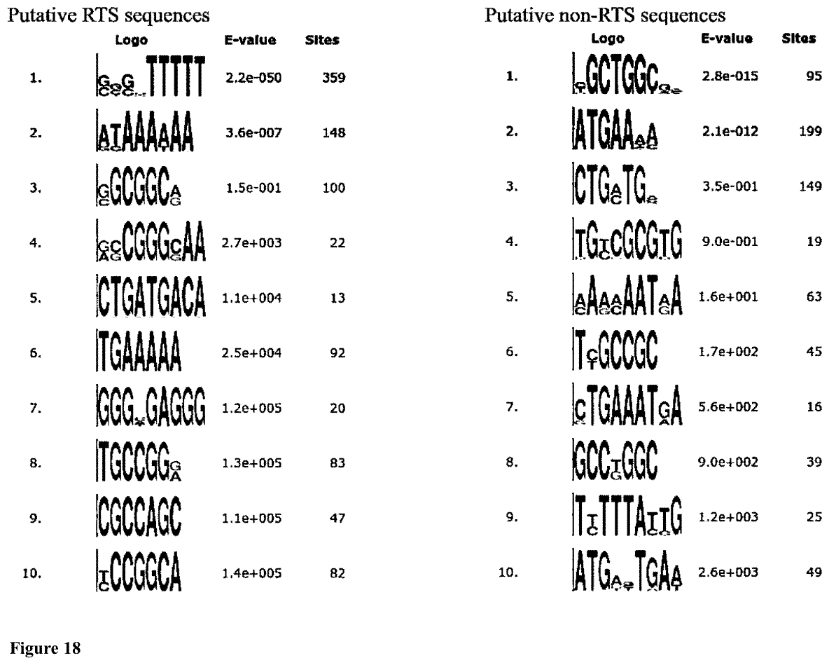

In some embodiments, a region with decreased folding energy or low folding energy comprises a ribosome termination structure (RTS). In some embodiments, an RTS is an RTS sequence. In some embodiments, an RTS sequence is provided in FIG. 18. In some embodiments, the region with decreased folding energy or low folding energy is an RTS. In some embodiments, the region comprises an RTS. In some embodiments, a region with decreased or low folding energy comprises increased secondary structure. In some embodiments, the secondary structure is an RTS. In some embodiments, the RTS is selected from TTTTT (SEQ ID NO: 44), X₃₉X₄₀X₄₁X₄₂TTTTT (SEQ ID NO: 66) wherein X₃₉ is G or C, X₄₀ is G or C, X₄₁ is G or C and X₄₂ is A, T, G, or C, X₁X₂AAAX₃AA (SEQ ID NO: 45) wherein X₁ is selected from A and G, X₂ is selected from T and C and X₃ is selected from A and T, X₄GCGGCX₅ (SEQ ID NO: 46) wherein X₄ is G or C and X₅ is A or G, X₆X₇CGGGX₈AA (SEQ ID NO: 47) wherein X₆ is G or A, X₇ is C or G and X₈ is C or G, CTGATGACA (SEQ ID NO: 48), TGAAAAA (SEQ ID NO: 49), GGGX₉GAGGG (SEQ ID NO: 50) wherein X₉ is A, T, C or G, TGCCGGX₁₀ (SEQ ID NO: 51) wherein X₁₀ is G or A, CGCCAGC (SEQ ID NO: 52) and X₁₁CCGGCA (SEQ ID NO: 53) wherein X₁₁ is T or C. In some embodiments, the RTS is SEQ ID NO: 44. In some embodiments, the RTS is SEQ ID NO: 45. In some embodiments, the RTS is SEQ ID NO: 66. In some embodiments, SEQ ID NO: 65 comprises SEQ ID NO: 44. In some embodiments, the RTS is SEQ ID NO: 46. In some embodiments, the RTS is SEQ ID NO: 47. In some embodiments, the RTS is SEQ ID NO:48. In some embodiments, the RTS is SEQ ID NO: 49. In some embodiments, the RTS is SEQ ID NO: 50. In some embodiments, the RTS is SEQ ID NO: 51. In some embodiments, the RTS is SEQ ID NO: 52. In some embodiments, the RTS is SEQ ID NO: 53. In some embodiments, the SEQ ID NO: 45 is ATAAAAAA (SEQ ID NO: 54). In some embodiments, the RTS is SEQ ID NO: 54. In some embodiments, the RTS is selected from SEQ ID NO: 45-53. In some embodiments, the mutation is within the RTS. In some embodiments, the mutation produces a sequence that is not an RTS. In some embodiments, the mutation produces a region that is devoid of an RTS. In some embodiments, the RTS is selected from SEQ ID NO: 44-45. In some embodiments, the RTS is selected from SEQ ID NO: 45 and 66. In some embodiments, the RTS is selected from SEQ ID NO: 54 and 66.

In some embodiments, a region with increased folding energy or high folding energy comprises a non-RTS. In some embodiments, a non-RTS is a non-RTS sequence. In some embodiments, a non-RTS sequence is provided in FIG. 18. In some embodiments, the region with increased folding energy or high folding energy is a non-RTS. In some embodiments, the region comprises a non-RTS. In some embodiments, a region with increased or high folding energy comprises decreased secondary structure. In some embodiments, the secondary structure is an RTS. In some embodiments, the non-RTS is selected from GCTGGX₁₂ (SEQ ID NO: 55) wherein X₁₂ is selected from C and T, ATTGAAX₁₃X₁₄ (SEQ ID NO: 56) wherein X₁₃ is A, T or C and X₁₄ is A or C, CTGX₁₅TGX₁₆ (SEQ ID NO: 57) wherein X₁₅ is A or C and X₁₆ is A, C or G, X₁₇GX₁₈X₁₉GCGX₂₀G (SEQ ID NO: 58) wherein X₁₇ is T or C, X₁₈ is T or C, X₁₉ is C or G, X₂₀ is T or C, X₂₁AX₂₂X₂₃AATX₂₄A (SEQ ID NO: 59) wherein X₂₁ is A or C, X₂₂ is A or G, X₂₃ is A or C, X₂₄ is A or G, TX₂₅GCCGC (SEQ ID NO: 60) wherein X₂₅ is C or T, X₂₆TGAAATX₂₇A (SEQ ID NO: 61) wherein X₂₆ is C or G and X₂₇ is G or A, GCCX₂₈GGC (SEQ ID NO: 62) wherein X₂₈ is T or G, TX₂₉TTTAX₃₀X₃₁G (SEQ ID NO: 63) wherein X₂₉ is T or C, X₃₀ is T or C, X₃₁ is T or C, and ATGX₃₂X₃₃TX₃₄AX₃₅ (SEQ ID NO: 64) wherein X₃₂ is A, G or T, X₃₃ is G, C or T, X₃₄ is G or A and X₃₅ is A or T. In some embodiments, the non-RTS is SEQ ID NO: 55. In some embodiments, the non-RTS is SEQ ID NO: 56. In some embodiments, the non-RTS is SEQ ID NO: 57. In some embodiments, the non-RTS is SEQ ID NO: 58. In some embodiments, the non-RTS is SEQ ID NO: 59. In some embodiments, the non-RTS is SEQ ID NO: 60. In some embodiments, the non-RTS is SEQ ID NO: 61. In some embodiments, the non-RTS is SEQ ID NO: 62. In some embodiments, the non-RTS is SEQ ID NO: 63. In some embodiments, the non-RTS is SEQ ID NO: 64. In some embodiments, SEQ ID NO: 55 is X₃₆GCTGGX₁₂X₃₇X₃₈ (SEQ ID NO: 65), wherein X₃₆ is C, T or G, X₁₂ is C or T, X₃₇ is G, C or A and X₃₈ is C, T, G or A. In some embodiments, the non-RTS is SEQ ID NO: 65. In some embodiments, the non-RTS is selected from SEQ ID NO: 55-56. In some embodiments, the non-RTS is selected from SEQ ID NO: 65-56. In some embodiments, the mutation is in a non-RTS sequence. In some embodiments, the mutation converts the non-RTS into an RTS. In some embodiments, the mutation produces a sequence devoid of a non-RTS sequence. In some embodiments, the mutation converts a non-RTS sequence into a sequence comprising secondary structure.

In some embodiments, the third region comprises at least one of:
i. a fragment of a naturally occurring sequence 3′ to a stop codon comprising a mutation that increases folding energy of the region or of RNA encoded by the region; or
ii. an artificial sequence configured such that a folding energy of the region or of RNA encoded by the region is above a predetermined threshold.

In some embodiments, the third region comprises at least one of:
i. a fragment of a naturally occurring sequence 3′ to a stop codon comprising a mutation that decreases folding energy of the region or of RNA encoded by the region; or
ii. an artificial sequence configured such that a folding energy of the region or of RNA encoded by the region is below a predetermined threshold.

In some embodiments, the third region comprises a fragment of a naturally occurring sequence 3′ to a stop codon comprising a mutation that increases folding energy of the region or of RNA encoded by the region. In some embodiments, the third region comprises an artificial sequence configured such that a folding energy of the region or of RNA encoded by the region is above a predetermined threshold. In some embodiments, the third region comprises a fragment of a naturally occurring sequence 3′ to a stop codon comprising a mutation that decreases folding energy of the region or of RNA encoded by the region. In some embodiments, the third region comprises an artificial sequence configured such that a folding energy of the region or of RNA encoded by the region is below a predetermined threshold.

Mutations that increase or decrease local folding energy are well known in the art. Whether a mutation increase or decreases local folding energy can be determined by modeling or empirically. Methods of determining local folding energy are well known in the art and any such method may be employed. Methods are also provided herein and any of these methods may be employed. In some embodiments, the method comprises determining the local folding energy for a region, generating at least one mutation in the region, determining the local folding energy in the mutated region and selecting the mutation if it increases the local folding energy. In some embodiments, the method comprises determining the local folding energy for a region, generating at least one mutation in the region, determining the local folding energy in the mutated region and selecting the mutation if it decreases the local folding energy. In some embodiments, determining local folding energy comprises inputting the sequence into a folding program. In some embodiments, a folding program is a program that predicts RNA folding. In some embodiments, a folding program is a program that models RNA folding. In some embodiments, a folding program provides a folding energy for a sequence. In some embodiments, the folding energy is local folding energy. In some embodiments, local is over a given window. In some embodiments, the window is 40 nt. In some embodiments, the sequence is the sequence of the region. Examples of folding programs are well known in the art and include for example, Mfold, RNAfold, RNA123, RNAshapes, RNAstructure, RNAstructureWeb, RNAslider and UNAFold to name but a few. In some embodiments, local folding energy is determined with RNAfold. Once the local folding energy is found for a given sequence over a given window various mutations can be tested for their effect on local folding energy. A mutation that increases folding energy or a mutation that decreases folding energy can be selected. Multiple mutations can be tested at once, or one at a time. When the folding architecture of a window is known, the mutations can be designed rationally, as generating mismatches in areas of secondary structure will reduce the secondary structure and thus increase local folding energy. Similarly, generating secondary structure where there was none will decrease local folding energy. Since the G-C bonds is stronger than the T-A bond, substituting one for the other can decrease local folding energy (T-A to G-C) or increase local folding energy (G-C to T-A). The predicted local folding energy can be compared to a null model to detect/predict meaningful levels of folding energy changes. A mutant region can also be tested empirically by methods such as are described herein. The region can be inserted into a dual reporter plasmid between the two reporters. The dual reporter may be for example GFP and RFP. Changes in expression of the downstream (e.g., RFP) and the upstream reporter (e.g., GFP) can be monitored. Increases in expression of the downstream reporter indicate that the folding energy just after the stop codon of the upstream reporter has been increased (i.e., weaker folding) leading to increased re-initiation. Decreases in expression of the downstream reporter indicate that the folding energy just after the stop codon of the upstream reporter has been decreased (i.e., stronger folding) leading to decreased re-initiation. Changes in expression of the upstream (e.g., GFP) reporter can be monitored. Increases in expression of the upstream reporter indicate that the folding energy just after the stop codon has been decreased (i.e., stronger folding) leading to better selection of the stop codon or regions upstream of it. Decreases in expression of the upstream reporter indicate that the folding energy has been increased (i.e., weaker folding) leading to worse selection of the stop codon or regions upstream of it.

In some embodiments, the region comprises a fragment of a naturally occurring sequence 3′ to a stop codon. In some embodiments, the fragment comprises an RTS. In some embodiments, the fragment comprises a non-RTS. In some embodiments, the sequence 3′ to a stop codon is a 3′ UTR. In some embodiments, the naturally occurring sequence is proximal to a stop codon. In some embodiments, the region 3′ to a stop codon comprises a start codon for another coding sequence. It will thus be understood that a sequence can be a 3′ UTR of one gene, but actually be a coding region for another gene. In some embodiments, the region comprises a fragment of a naturally occurring 3′ UTR. In some embodiments, the region consists of a fragment of a naturally occurring 3′ UTR. In some embodiments, the fragment or RNA encoded by the fragment comprises a folding energy that is above a predetermined threshold. In some embodiments, the nucleic acid molecule comprises the fragment and is devoid of the rest of the 3′ UTR. In some embodiments, the nucleic acid molecule comprises the fragment but does not comprise the entire 3′ UTR. In some embodiments, the nucleic acid molecule comprises the fragment, but does not comprise more than 50, 75, 100, 125, 150, 175, 200, 250, 300, 350, 400, 450, 500, 600, 700, 800, 900 or 1000 bp of the 3′ UTR or sequence 3′ to the stop codon. Each possibility represents a separate embodiment of the invention. In some embodiments, the fragment is from 10-50, 10-75, 10-100, 10-150, 10-200, 10-250, 10-300, 10-350, 10-400, 10-450, 10-500, 10-600, 10-700, 10-800, 10-900, 10-1000, 20-50, 20-75, 20-100, 20-150, 20-200, 20-250, 20-300, 20-350, 20-400, 20-450, 20-500, 20-600, 20-700, 20-800, 20-900, 20-1000, 25-50, 25-75, 25-100, 25-150, 25-200, 25-250, 25-300, 25-350, 25-400, 252-450, 25-500, 25-600, 25-700, 25-800, 25-900, 25-1000, 30-50, 30-75, 30-100, 30-150, 30-200, 30-250, 30-300, 30-350, 30-400, 30-450, 30-500, 30-600, 30-700, 30-800, 30-900, 30-1000, 40-50, 40-75, 40-100, 40-150, 40-200, 40-250, 40-300, 40-350, 40-400, 40-450, 40-500, 40-600, 40-700, 40-800, 40-900, 40-1000, 50-75, 50-100, 50-150, 50-200, 50-250, 50-300, 50-350, 50-400, 50-450, 50-500, 50-600, 50-700, 50-800, 50-900, or 50-1000 nucleotides in length.

In some embodiments, the UTR is a prokaryotic UTR. In some embodiments, the UTR is a bacterial UTR. In some embodiments, the UTR is a eukaryotic UTR. In some embodiments, the UTR is untranslated for a first coding sequence but contains a coding sequence for a second gene and thus is translated. In some embodiments, the fragment comprises a UTR and a 5′ end of another coding sequence.

In some embodiments, the region comprises a fragment of a naturally occurring 3′ UTR comprising a mutation that increases folding energy of the region or of RNA encoded by the region. In some embodiments, the fragment comprises a mutation that increases folding energy of the region or of RNA encoded by the region. It will be understood by a skilled artisan that RNA readily assumes a secondary structure and that the more structured the RNA the lower the folding energy. As the invention is concerned with the folding energy and secondary structure of mRNA as it is translated, the region may be considered to have a folding energy in so much as the molecule is an RNA or the region may be considered to encode an RNA with a folding energy in so much as the molecule is a DNA molecule. In some embodiments, the folding energy is Gibbs free energy. In some embodiments, the Gibbs free energy is RNA secondary structure folding Gibbs free energy. In some embodiments, increasing folding energy comprises decreasing RNA secondary structure. In some embodiments, increasing folding energy comprises decreasing RNA folding.

In some embodiments, increase is an increase of at least 1, 2, 3, 4, 5, 7, 10, 20, 25, 30, 40, 50, 60, 70, 80, 90, 100, 125, 150, 175, 200, 250, 300, 350, 400, 450, or 500% in folding energy. Each possibility represents a separate embodiment of the invention. In some embodiments, increase is an increase of at least 0.1, 0.5, 1, 1.5, 2, 2.5, 3, 3.5, 4, 4.5, 5, 5.5, 6, 6.5, 7, 7.5, 8, 8.5, 9, 9.5 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, or 35 kcal/mol or kcal/mol/40 bp. Each possibility represents a separate embodiment of the invention.

In some embodiments, a mutation is at least one mutation. In some embodiments, a mutation is at least 2, 3, 4, 5, 6, 7 8, 9, 10, 11, 12, 13, 14, 15, 16, 17, 18, 19, 20, 21, 22, 23, 24, 25, 26, 27, 28, 29, 30, 31, 32, 33, 34, or 35 mutations. Each possibility represents a separate embodiment of the invention. A mutation may alter folding by changing the base pairing that can occur between nucleotides in the region. Programs for assessing RNA folding and secondary structure are well known and any method of evaluating folding energy change may be used. Examples of such programs include, but are not limited to, RNAfold (rna.tbi.univie.ac.at/cgi-bin/RNAwebsuite/RNAfold.cgi), RNAstructureWeb (rna.urmc.rochester.edu/RNAstructureweb), and RNAslider (tbi.univie.ac.at/RNA/ViennaRNA/doc/html/group_mfe_window.html). In some embodiments, a change in folding energy is measured as the change in local folding energy (ΔLFE). In some embodiments, a change in folding energy is measured as the change in RNA secondary structure folding Gibbs free energy.

It will be understood by a skilled artisan that the measure of folding energy is generally negative, and that an area with complex secondary structure, i.e., abundant folding, will have a very low, negative folding energy. Thus, increasing folding energy is decreasing secondary structure complexity and decreasing folding. In some embodiments, the substitution or mutation increases folding energy of the region or RNA encoded by the region to above a predetermined threshold. In some embodiments, the predetermined threshold is −5 kcal/mol/40 bp. In some embodiments, the threshold is a statistically significant increase. In some embodiments, the threshold is a statistically significant decrease. In some embodiments, the threshold is a value above which the difference as compared to the already existing folding energy would be significant. In some embodiments, the threshold is a level that is statistically significant as compared to a null model for folding energy of the region.

In some embodiments, the region comprises at least a portion of a second coding sequence. In some embodiments, the region comprises at least a portion of the second coding sequence. In some embodiments, the portion is a 5′ portion. In some embodiments, the region comprises the start codon of the second coding sequence. In some embodiments, the first coding sequence and the second coding sequence are overlapping. In some embodiments, the start codon of the second sequence is 5′ to the stop codon of the first sequence. In some embodiments, the region comprises coding sequence of the second sequence.

In some embodiments, the portion of the second coding sequence within the region comprises at least one codon substituted to a different codon. In some embodiments, the substitution increases folding energy of the region or of RNA encoded by the region. In some embodiments, the mutation is a synonymous mutation. In some embodiments, the region comprises at least one, at least 2, at least 3, at least 4, at least 5, at least 6, at least 7, at least 8, at least 9 or at least 10 codons substituted. Each possibility represents a separate embodiment of the invention. In some embodiments, the region comprises 1, 2, 3, 4, 5, 6, 7, 8, 9, or 10 codons substituted. Each possibility represents a separate embodiment of the invention. In some embodiments, all codons which can be substituted to a synonymous codon that increases the folding energy of the region or of RNA encoded by the region are substituted.

In some embodiments, the another codon is a synonymous codon. In some embodiments, a codon is substituted to a synonymous codon. In some embodiments, the substitution is a silent substitution. In some embodiments, the substitution is a mutation. In some embodiments, a codon is mutated to another codon. In some embodiments, the other codon is a synonymous codon. In some embodiments, the mutation is a silent mutation.

The term “codon” refers to a sequence of three DNA or RNA nucleotides that correspond to a specific amino acid or stop signal during protein synthesis. The codon code is degenerate, in that more than one codon can code for the same amino acid. Such codons that code for the same amino acid are known as “synonymous” codons. Thus, for example, CUU, CUC, CUA, CUG, UUA, and UUG are synonymous codons that code for Leucine. Synonymous codons are not used with equal frequency. In general, the most frequently used codons in a particular cell are those for which the cognate tRNA is abundant, and the use of these codons enhances the rate of protein translation. Conversely, tRNAs for rarely used codons are found at relatively low levels, and the use of rare codons is thought to reduce translation rate. “Codon bias” as used herein refers generally to the non-equal usage of the various synonymous codons, and specifically to the relative frequency at which a given synonymous codon is used in a defined sequence or set of sequences.

As used herein, the term “silent mutation” refers to a mutation that does not affect or has little effect on protein functionality. A silent mutation can be a synonymous mutation and therefore not change the amino acids at all, or a silent mutation can change an amino acid to another amino acid with the same functionality or structure, thereby having no or a limited effect on protein functionality.

In some embodiments, the region comprises at plurality of codons substituted to another codon. In some embodiments, each substitution increases folding energy of the region or RNA encoded by the region. In some embodiments, the plurality of mutations in combination increases folding energy of the region or RNA encoded by the region.

In some embodiments, at least 1, at least 2, at least 3, at least 4, at least 5, at least 10, at least 15, at least 20, at least 25, or at least 30 codons of the region have been substituted. Each possibility represents a separate embodiment of the present invention. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of all codons in the region have been substituted. Each possibility represents a separate embodiment of the present invention. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of codons in the region that have synonymous codons that increase the folding energy of the region have been substituted. Each possibility represents a separate embodiment of the present invention.

In some embodiments, all possible codons with the region are substituted to synonymous codons that increase folding energy of the region or RNA encoded by the region. In some embodiments, codons are substituted to synonymous codons to produce a region with the highest possible folding energy while maintaining the amino acid sequence of a peptide encoded by the region. In some embodiments, all possible combinations of synonymous mutations are examined and the combination with the highest folding energy is selected. In some embodiments, the region comprise synonymous codons substituted to increase folding energy to a maximum possible for the region.

In some embodiments, the region comprises an artificial sequence. In some embodiments, the region consists of an artificial sequence. In some embodiments, an artificial sequence is a sequence which is not found in nature. In some embodiments, an artificial sequence is a sequence with less than 100, 99, 97, 95, 92, 90, 85, 80, 75, 70, 65, 60, 55 or 50% homology to a naturally occurring sequence. Each possibility represents a separate embodiment of the invention.

In some embodiments, the artificial sequence is configured such that a folding energy of the region or of RNA encoded by the region is above a predetermined threshold. In some embodiments, the predetermined threshold is the limit below which the second coding sequence is insulated from ribosome re-initiation. In some embodiments, the predetermined threshold is the limit above which ribosome re-initiation at the second coding sequence occurs. In some embodiments, the predetermined threshold is the limit above which ribosome re-initiation at the second coding sequence is induced. In some embodiments, the predetermined threshold is the limit above which ribosome re-initiation at the second coding sequence is increased. In some embodiments, the threshold is −5 kcal/mol. In some embodiments, the threshold is −6 kcal/mol. In some embodiments, the threshold is −5 kcal/mol/40 bp. In some embodiments, the threshold is −6 kcal/mol/40 bp. In some embodiments, the threshold is a level which comprises a statistically significant difference as compared to a null model for folding energy for the region. In some embodiments, an RTS is a sequence directly downstream of the stop codon and with a local folding energy of below −6 kcal/mol/40 bp. In some embodiments, increased folding energy, high folding energy and/or decreased structure is above the threshold. In some embodiments, decreased folding energy, low folding energy and/or increased structure is below the threshold. In some embodiments, increased local folding energy causes re-initiation at the second coding sequence (e.g., the second start codon). In some embodiments, decreased local folding energy inhibits re-initiation at the second coding sequence (e.g., the second start codon).

In some embodiments, the region is devoid of an internal ribosome entry site (IRES). In some embodiments, the nucleic acid molecule is devoid of an IRES between the first coding sequence and the second coding sequence. In some embodiments, the nucleic acid molecule is devoid of an IRES between the at least two coding sequences. In some embodiments, the vector is devoid of an IRES between the first and second regions.

By another aspect, there is provided a nucleic acid molecule comprising a coding sequence and a region around a stop codon of the coding sequence, wherein the region or RNA encoded by the region comprises low or decreased folding energy.

By another aspect, there is provided an expression vector comprising a first region for insertion of a coding sequence; and a second region around the end of the first region, wherein the second region or RNA encoded by the second region comprising low or decreased folding energy.

In some embodiments, the region around the stop codon of the coding sequence is downstream of the stop codon. In some embodiments, the region around the end of the first region is downstream of the first region. In some embodiments, the region around the stop codon of the first coding sequence is the second region. In some embodiments, the end is the 3′ end.

In some embodiments, the coding sequence comprises a stop codon. In some embodiments, the region around the stop codon of the coding sequence is downstream of the stop codon. In some embodiments, the region is from the stop codon to 25, 30, 40, 50, 60, 70, 75, 80, 90, or 100 nucleotides downstream of the stop codon. Each possibility represents a separate embodiment of the invention. In some embodiments, the region is from the stop codon to 100 nucleotides downstream of the stop codon. In some embodiments, the region is from the stop codon to 75 nucleotides downstream of the stop codon. In some embodiments, the region is from the stop codon to 50 nucleotides downstream of the stop codon. In some embodiments, the region is from the stop codon to 40 nucleotides downstream of the stop codon. In some embodiments, the region includes the stop codon. In some embodiments, the region excludes the stop codon. It will be understood that for the purposes of numbering the third base of the stop codon will be considered base zero and so the first base after the stop codon will be considered base +1 relative to the stop codon, or base 1 downstream of the stop codon. In some embodiments, the region is from 1 to 25, 1 to 30, 1 to 40, 1 to 50, 1 to 60, 1 to 70, 1 to 75, 1 to 80, 1 to 90, or 1 to 100 nucleotides downstream of the stop codon. Each possibility represents a separate embodiment of the invention. In some embodiments, the region is from 1 to 100 nucleotides downstream of the stop codon. In some embodiments, the region is from 1 to 75 nucleotides downstream of the stop codon. In some embodiments, the region is from 1 to 50 nucleotides downstream of the stop codon. In some embodiments, the region is from 1 to 40 nucleotides downstream of the stop codon.

In some embodiments, the codons covered by the ribosome while it is reading the stop codon are not part of the region. In some embodiments, the region begins at 7 nucleotides downstream of the stop codon. It will be known by a skilled artisan that while the ribosome is reading the stop codon it will also be covering the next two codons, which is the next six nucleotides. As these nucleotides will be covered, they will not be free to interact with the region and will not be able to form secondary structure. In some embodiments, the region is from 7 to 100, 7 to 90, 7 to 80, 7 to 75, 7 to 70, 7 to 60, 7 to 50, 7 to 40, 7 to 30 or 7 to 25 nucleotides downstream of the stop codon. Each possibility represents a separate embodiment of the invention. In some embodiments, the region is from 7 to 100 nucleotides downstream of the stop codon. In some embodiments, the region is from 7 to 75 nucleotides downstream of the stop codon. In some embodiments, the region is from 7 to 50 nucleotides downstream of the stop codon. In some embodiments, the region is from 7 to 40 nucleotides downstream of the stop codon. In some embodiments, the region is from 9 to 100, 9 to 90, 9 to 80, 9 to 75, 9 to 70, 9 to 60, 9 to 50, 9 to 40, 9 to 30 or 9 to 25 nucleotides downstream of the stop codon. Each possibility represents a separate embodiment of the invention. In some embodiments, the region is from 9 to 100 nucleotides downstream of the stop codon. In some embodiments, the region is from 9 to 75 nucleotides downstream of the stop codon. In some embodiments, the region is from 9 to 50 nucleotides downstream of the stop codon. In some embodiments, the region is from 9 to 40 nucleotides downstream of the stop codon. In some embodiments, the region is from 5 to 100, 5 to 90, 5 to 80, 5 to 75, 5 to 70, 5 to 60, 5 to 50, 5 to 40, 5 to 30 or 5 to 25 nucleotides downstream of the stop codon. Each possibility represents a separate embodiment of the invention. In some embodiments, the region is from 5 to 100 nucleotides downstream of the stop codon. In some embodiments, the region is from 5 to 75 nucleotides downstream of the stop codon. In some embodiments, the region is from 5 to 50 nucleotides downstream of the stop codon. In some embodiments, the region is from 5 to 40 nucleotides downstream of the stop codon.

In some embodiments, the region comprises:
a. a fragment of a naturally occurring sequence 3′ to a stop codon comprising a mutation that decreases folding energy of the region or RNA encoded by the region; or
b. an artificial sequence configured such that a folding free energy of the region or RNA encoded by the region is below a predetermined threshold.

In some embodiments, the region comprises:
a. a fragment of a naturally occurring sequence 3′ to a stop codon comprising a mutation that increases folding energy of the region or RNA encoded by the region; or
b. an artificial sequence configured such that a folding free energy of the region or RNA encoded by the region is above a predetermined threshold.

In some embodiments, the second region comprises:
a. a fragment of a naturally occurring sequence 3′ to a stop codon comprising a mutation that decreases folding energy of the region or RNA encoded by the region; or
b. an artificial sequence configured such that a folding free energy of the region or RNA encoded by the region is below a predetermined threshold.

In some embodiments, the second region comprises:
a. a fragment of a naturally occurring sequence 3′ to a stop codon comprising a mutation that increases folding energy of the region or RNA encoded by the region; or
b. an artificial sequence configured such that a folding free energy of the region or RNA encoded by the region is above a predetermined threshold.

In some embodiments, the region comprises a fragment of a naturally occurring sequence 3′ to a stop codon. In some embodiments, the sequence 3′ to a stop codon is a 3′ UTR. In some embodiments, the region 3′ to a stop codon comprises a start codon for another coding sequence. In some embodiments, the region comprises a fragment of a naturally occurring 3′ UTR. In some embodiments, the region consists of a fragment of a naturally occurring 3′ UTR. In some embodiments, the fragment or RNA encoded by the fragment comprises a folding energy that is below a predetermined threshold. In some embodiments, the nucleic acid molecule comprises the fragment and is devoid of the rest of the 3′ UTR. In some embodiments, the nucleic acid molecule comprises the fragment but does not comprise the entire 3′ UTR. In some embodiments, the nucleic acid molecule comprises the fragment, but does not comprise more than 50, 75, 100, 125, 150, 175, 200, 250, 300, 350, 400, 450, 500, 600, 700, 800, 900 or 1000 bp of the 3′ UTR or sequence 3′ to the stop codon. Each possibility represents a separate embodiment of the invention. In some embodiments, the fragment is from 10-50, 10-75, 10-100, 10-150, 10-200, 10-250, 10-300, 10-350, 10-400, 10-450, 10-500, 10-600, 10-700, 10-800, 10-900, 10-1000, 20-50, 20-75, 20-100, 20-150, 20-200, 20-250, 20-300, 20-350, 20-400, 20-450, 20-500, 20-600, 20-700, 20-800, 20-900, 20-1000, 25-50, 25-75, 25-100, 25-150, 25-200, 25-250, 25-300, 25-350, 25-400, 252-450, 25-500, 25-600, 25-700, 25-800, 25-900, 25-1000, 30-50, 30-75, 30-100, 30-150, 30-200, 30-250, 30-300, 30-350, 30-400, 30-450, 30-500, 30-600, 30-700, 30-800, 30-900, 30-1000, 40-50, 40-75, 40-100, 40-150, 40-200, 40-250, 40-300, 40-350, 40-400, 40-450, 40-500, 40-600, 40-700, 40-800, 40-900, 40-1000, 50-75, 50-100, 50-150, 50-200, 50-250, 50-300, 50-350, 50-400, 50-450, 50-500, 50-600, 50-700, 50-800, 50-900, or 50-1000 nucleotides in length.

In some embodiments, the region comprises a fragment of a naturally occurring 3′ UTR comprising a mutation that decreases folding energy of the region or of RNA encoded by the region. In some embodiments, the fragment comprises a mutation that decreases folding energy of the region or of RNA encoded by the region. In some embodiments, decreases folding energy comprises increasing RNA secondary structure. In some embodiments, decreases folding energy comprises increasing RNA folding.

It will be understood by a skilled artisan that the measure of folding energy is generally negative, and that an area with complex secondary structure, i.e., abundant folding, will have a very low, negative folding energy. Thus, decreasing folding energy is increasing secondary structure complexity and increasing folding. In some embodiments, the substitution or mutation decreases folding energy of the region or RNA encoded by the region to above a predetermined threshold. In some embodiments, the predetermined threshold is −5 kcal/mol/40 bp.

In some embodiments, decrease is a decrease of at least 1, 2, 3, 4, 5, 7, 10, 20, 25, 30, 40, 50, 60, 70, 80, 90, 100, 125, 150, 175, 200, 250, 300, 350, 400, 450, or 500% in folding energy. Each possibility represents a separate embodiment of the invention. In some embodiments, decrease is a decrease of at least 0.1, 0.5, 1, 1.5, 2, 2.5, 3, 3.5, 4, 4.5, 5, 5.5, 6, 6.5, 7, 7.5, 8, 8.5, 9, 9.5 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, or 35 kcal/mol or kcal/mol/40 bp. Each possibility represents a separate embodiment of the invention.

In some embodiments, the region comprises an artificial sequence. In some embodiments, the artificial sequence is configured such that a folding energy of the region or of RNA encoded by the region is below a predetermined threshold. In some embodiments, the threshold is −5 kcal/mol. In some embodiments, the threshold is −5 kcal/mol/40 bp. In some embodiments, the threshold is −6 kcal/mol. In some embodiments, the threshold is −6 kcal/mol/40 bp. In some embodiments, the region insulates against downstream ribosome re-initiation. In some embodiments, the region increases ribosome termination at the stop codon. In some embodiments, the second region increases ribosome termination at a stop codon of the inserted coding sequence. In some embodiments, the second region increases ribosome termination at the 3′ end of the first region. In some embodiments, the region increases mRNA dissociation of a ribosome at the stop codon. In some embodiments, the second region increases mRNA dissociation of a ribosome at a stop codon of the inserted coding sequence. In some embodiments, the second region increases mRNA dissociation of a ribosome at the 3′ end of the first region. In some embodiments, dissociation is from the stop codon. In some embodiments, dissociation is from the nucleic acid molecule. In some embodiments, dissociation is from an RNA encoded by the nucleic acid molecule. In some embodiments, the RNA is an mRNA.

In some embodiments, the region or the second region is devoid of Rho-independent transcriptional terminators. In some embodiments, the region or the second region is devoid of Rho-independent transcription terminators. In some embodiments, the nucleic acid molecule is devoid of a Rho-independent transcriptional terminator. In some embodiments, the nucleic acid molecule is devoid of a Rho-independent transcriptional terminator after the coding sequence. In some embodiments, the nucleic acid molecule is devoid of a Rho-independent transcriptional terminator proximal to the coding sequence. In some embodiments, the vector is devoid of a Rho-independent transcriptional terminator. In some embodiments, the vector is devoid of a Rho-independent transcriptional terminator after the first region. In some embodiments, the vector is devoid of a Rho-independent transcriptional terminator proximal to the first region. In some embodiments, the Rho-independent transcriptional terminator comprises SEQ ID NO: 44. In some embodiments, the Rho-independent transcriptional terminator consists of SEQ ID NO: 44. In some embodiments, the Rho-independent transcriptional terminator is SEQ ID NO: 44.

In some embodiments, the first region comprises a first coding sequence. In some embodiments, the first coding sequence comprises a stop codon. In some embodiments, the second region is proximal to the stop codon. In some embodiments, the second region comprises a second coding sequence. In some embodiments, the second coding sequence comprises a translational start site (TSS). In some embodiments, the TSS is a start codon. In some embodiments, the TSS of the second coding sequence is proximal to the first region. In some embodiments, the TSS of the second coding sequence is proximal to an end of the first region. In some embodiments, the end is the 3′ end. In some embodiments, the end is a 5′ end.

In some embodiments, a region configured for insertion of a coding sequence is a multiple cloning site (MCS). MCSs are region with sequences that can be cleaved by restriction enzymes. MCSs contain multiple such sequences, that can be cleaved by different restriction enzymes. This allows for insertion of sequences that have also been cut by these, or compatible restriction enzymes. MCSs are well known in the art and any sequence of a multiple cloning site may be used.

By another aspect, there is provided an expression vector comprising a nucleic acid molecule of the invention.

By another aspect, there is provided a method for producing a nucleic acid molecule optimized for expression of a protein encoded by a second coding sequence proximal to a stop codon of a first coding sequence, the method comprising: generating a region around the stop codon of the first coding sequence, wherein the region or RNA encoded by the region has increased or high folding energy.

In some embodiments, the nucleic acid molecule is an RNA molecule and comprises both coding sequences. In some embodiments, the nucleic acid molecule is a DNA molecule encoding a single RNA molecule comprising both coding sequences. In some embodiments, the first coding sequence encodes a protein. In some embodiments, the second coding sequence encodes a protein. In some embodiments, the first coding sequence encodes a first protein, and the second coding sequence encodes a second protein. In some embodiments, the nucleic acid molecule is devoid of an IRES between the first sequence encoding a first protein and the second sequence encoding the second protein.

In some embodiments, the TSS or the start codon of the second coding sequence is proximal to the stop codon of the first coding sequence. In some embodiments, the TSS or the start codon of the second coding sequence is proximal to the 3′ end of the first coding sequence. In some embodiments, the region is a region such as is described hereinabove. In some embodiments, the region comprises at least a portion of the second coding sequence. In some embodiments, the method is for optimizing production of the second protein without a mutation in its amino acid sequence and the region comprises synonymous mutations of the second coding region.

In some embodiments, generating a region comprises inserting the region around the stop codon. In some embodiments, generating a region comprises introducing a mutation. In some embodiments, generating a region comprises intruding a mutation into a region around the stop codon.

In some embodiments, the method is for producing a nucleic acid molecule with increased ribosome translational re-initiation at the second coding region. In some embodiments, the method is for producing a nucleic acid molecule with increased ribosome translational re-initiation at a TSS or start codon of the second coding region.

By another aspect, there is provided a method for producing a nucleic acid molecule optimized for expressing a first protein, the method comprising, generating a region around a stop codon of a coding sequence encoding the first protein, wherein the region or RNA encoded by the region comprises decreased or low folding energy.

In some embodiments, generating a region comprises inserting the region around the stop codon. In some embodiments, generating a region comprises introducing a mutation. In some embodiments, generating a region comprises intruding a mutation into a region around the stop codon.

In some embodiments, the method is for producing a nucleic acid molecule with increased ribosome termination at the stop codon of a coding sequence. In some embodiments, the method is for producing a nucleic acid molecule with increased mRNA dissociation of a ribosome at the stop codon of a coding sequence. In some embodiments, the method is for producing a nucleic acid molecule with increased ribosome termination at the stop codon of a coding sequence encoding the first protein. In some embodiments, the method is for producing a nucleic acid molecule with increased mRNA dissociation of a ribosome at the stop codon of a coding sequence encoding the first protein. In some embodiments, dissociation is from the stop codon. In some embodiments, dissociation is from the nucleic acid molecule. In some embodiments, dissociation is from an RNA encoded by the nucleic acid molecule. In some embodiments, the RNA is an mRNA.

In some embodiments, optimizing is optimizing expression. In some embodiments, optimizing is optimizing protein expression. In some embodiments, optimizing is optimizing translation. In some embodiments, optimizing is optimizing in a target cell. In some embodiments, the target cell is a prokaryotic cell. In some embodiments, the target cell is a bacterial cell. In some embodiments, the target cell is a eukaryotic cell. In some embodiments, the eukaryote is a mammal. In some embodiments, the mammal is a human.

In some embodiments, the nucleic acid molecule is a vector. In some embodiments, the vector is an expression vector. In some embodiments, the nucleic acid molecule further comprises at least one regulatory element. In some embodiments, the at least one regulatory element is operatively linked to the first coding sequence encoding the first protein. In some embodiments, the at least one regulatory element is operatively linked to the second coding sequence encoding the second protein. In some embodiments, the at least one regulatory element is operatively linked to the first coding region and not the second coding region, wherein translation and/or transcription of the first coding sequence causes translation and/or transcription of the second coding sequence.

In some embodiments, the nucleic acid molecule is genomic DNA the introducing a mutation comprises genome editing. In some embodiments, the introducing a mutation is site-directed mutagenesis. In some embodiments, introducing a mutation is generating a sequence with the mutation. In some embodiments, introducing a mutation is providing a list of mutations within the region that increase or decrease the folding energy.

Methods of genome editing include, but are not limited to CRISPR, TALEN, Meganucleases and Zinc finger domain proteins. Any method of genome editing may be employed. Methods of nucleic acid mutagenesis are also well known, and any such method may be employed. It may be that rather than mutagenizing a molecule, a new molecule may be synthesized de novo that includes the mutation. Thus, introduction of the mutation is into a sequence and need not actually comprise producing the nucleic acid molecule.

By another aspect, there is provided a method of converting an overlapping gene pair into two non-overlapping gene, the method comprising:
a. receiving a sequence of the overlapping gene pair comprising a first coding sequence of a first gene of the gene pair and a second coding sequence of a second gene of the gene pair, wherein a start codon of the second coding sequence is within the first coding sequence;
b. inserting the second coding sequence proximal to, and not overlapping with, a stop codon of the first coding sequence;
c. producing around the stop codon of the first coding sequence a region, wherein the region or RNA encoded by the region comprises higher or increased folding energy;
   thereby converting an overlapping gene pair into two non-overlapping genes.

In some embodiments, the overlapping gene pair comprises a portion of the second coding sequence within the first coding sequence. In some embodiments, the overlapping gene pair comprises a portion of the second coding sequence that is outside of the first coding sequence. In some embodiments, the portion of the second coding sequence that is outside the first coding sequence is downstream from the first coding sequence. In some embodiments, the portion of the second coding sequence that is outside the first coding sequence is 3′ to the first coding sequence.

In some embodiments, inserting the second coding sequence comprises inserting the second coding sequence downstream to the first coding sequence. In some embodiments, inserting the second coding sequence comprises removing the portion of the second coding sequence that was outside of the first coding sequence. In some embodiments, the portion of the second coding sequence outside of the first coding sequence is replaced by the full second coding sequence that is inserted. In some embodiments, the start codon of the inserted second coding sequence is inserted proximal to the 3′ end or stop codon of the first coding sequence.

In some embodiments, producing the region comprises at least one of:
i. inserting a fragment of a naturally occurring sequence 3′ to a stop codon comprising a mutation that increases folding energy of the region or of RNA encoded by the region;
ii. mutating at least one codon of the inserted second coding region to a different codon, wherein the substitution increases folding energy of the region or of RNA encoded by the region; or
iii. inserting an artificial sequence configured such that a folding energy of the region or of RNA encoded by the region is above a predetermined threshold.

In some embodiments, the mutation is a synonymous mutation. In some embodiments, the mutation within the second coding region is a synonymous mutation. In some embodiments, the inserted coding region encodes the same amino acid sequence of the second coding region as part of the overlapping gene pair. In some embodiments, producing is inserting the region. In some embodiments, producing comprises mutating an already existing sequence.

According to another aspect, there is provided a computer program product comprising a non-transitory computer-readable storage medium having program instructions embodied therewith, the program instructions executable by at least one hardware processor configured to perform a method of the invention.

According to another aspect, there is provided a computer program product comprising a non-transitory computer-readable storage medium having program instructions embodied therewith, the program instructions executable by at least one hardware processor configured to:
a. receive a sequence of a nucleic acid molecule comprising at least two coding sequences, wherein a start codon of a second coding sequence is proximal to a stop codon of a first coding sequence;
b. determine within a region around a stop codon of the first coding sequence at least one mutation that increases folding energy of the first region or RNA encoded by the first region; and
c. output a mutated sequence of the nucleic acid molecule comprising the at least one mutation, or a list of possible mutations in the region that increase folding energy of the region or RNA encoded by the region.

According to another aspect, there is provided a computer program product comprising a non-transitory computer-readable storage medium having program instructions embodied therewith, the program instructions executable by at least one hardware processor to execute a genetic-type machine learning algorithm configured to:
a. receive a nucleic acid molecule comprising a coding sequence;
b. determine within a region around a stop codon of the coding sequence at least one mutation that decreases folding energy of the region or RNA encoded by the region; and
c. output a mutated sequence of the nucleic acid molecule comprising the at least one mutation, or a list of possible mutations in the region that decrease folding energy of the region or RNA encoded by the region.

In some embodiments, the computer program product optimizes the region for expression of a protein encoded by the second coding sequence. In some embodiments, the computer program product optimizes the region for expression of a protein encoded by the first coding sequence. In some embodiments, the computer program product determines the combination of mutations that increases folding energy to a maximum while retaining the amino acid sequence of the encoded by the region. In some embodiments, the computer program product determines the combination of mutations that decreases folding energy to a minimum while retaining the amino acid sequence of the encoded by the region.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention may be described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Experimental Methods

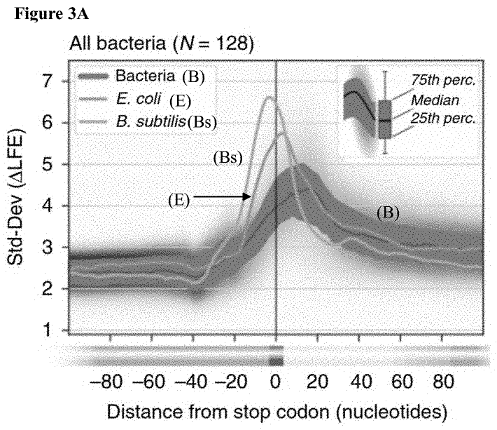

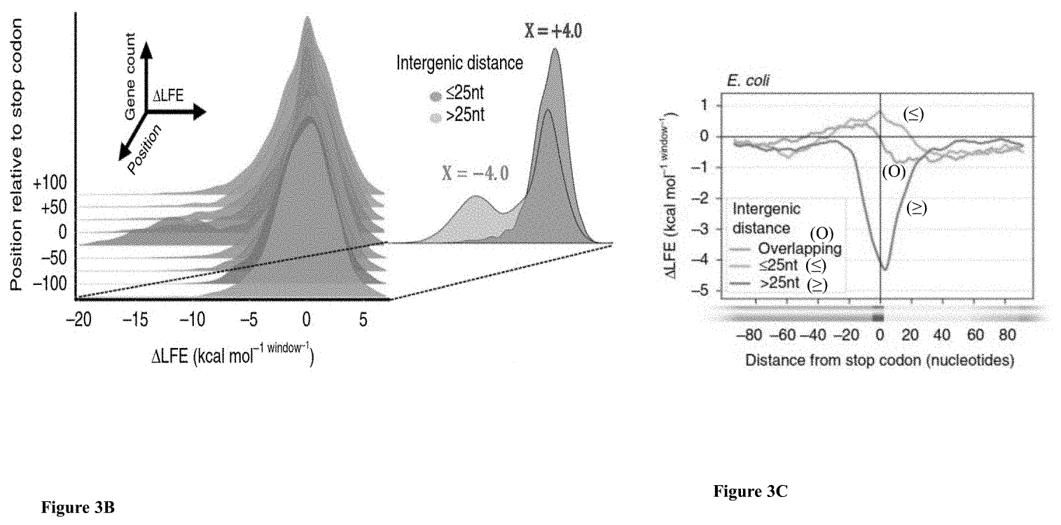

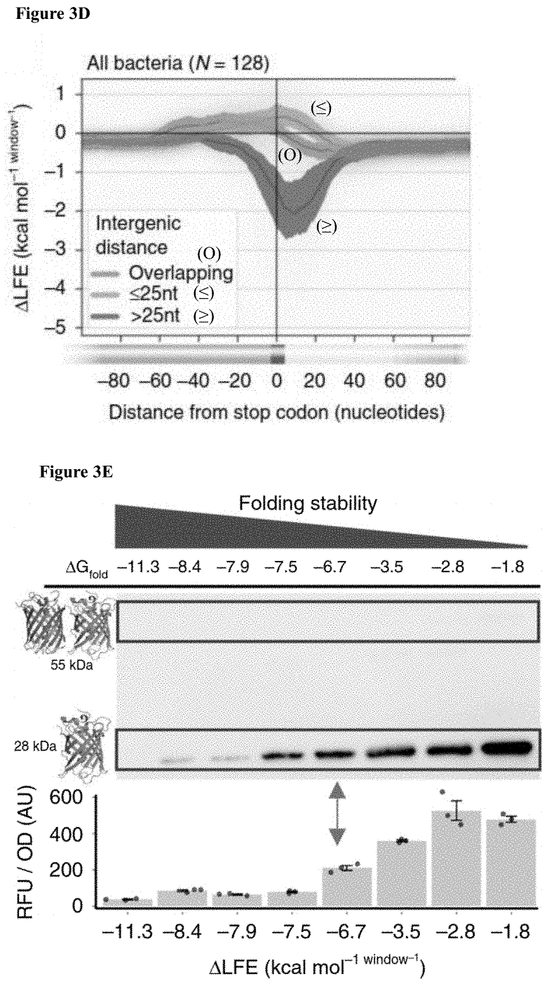

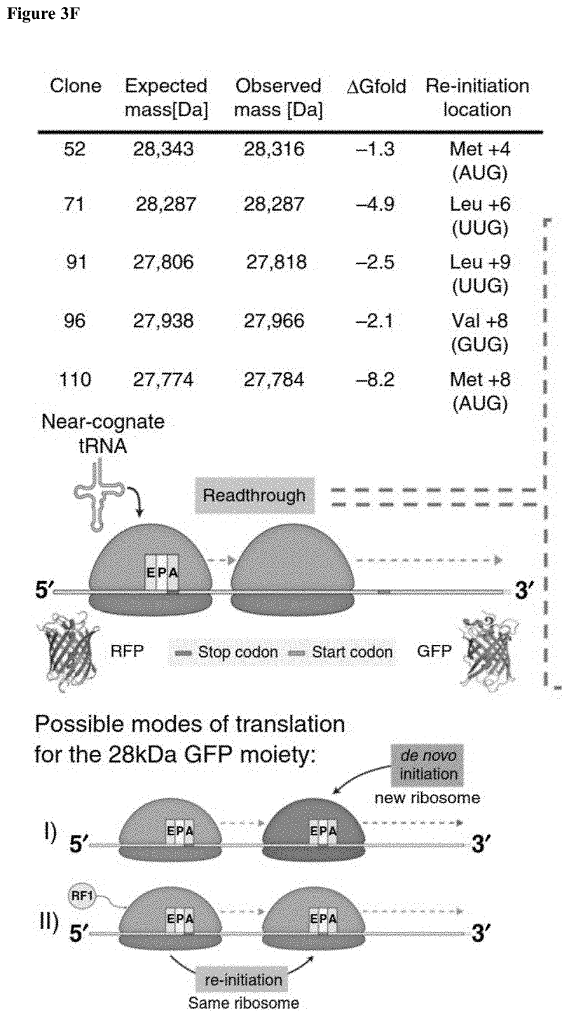

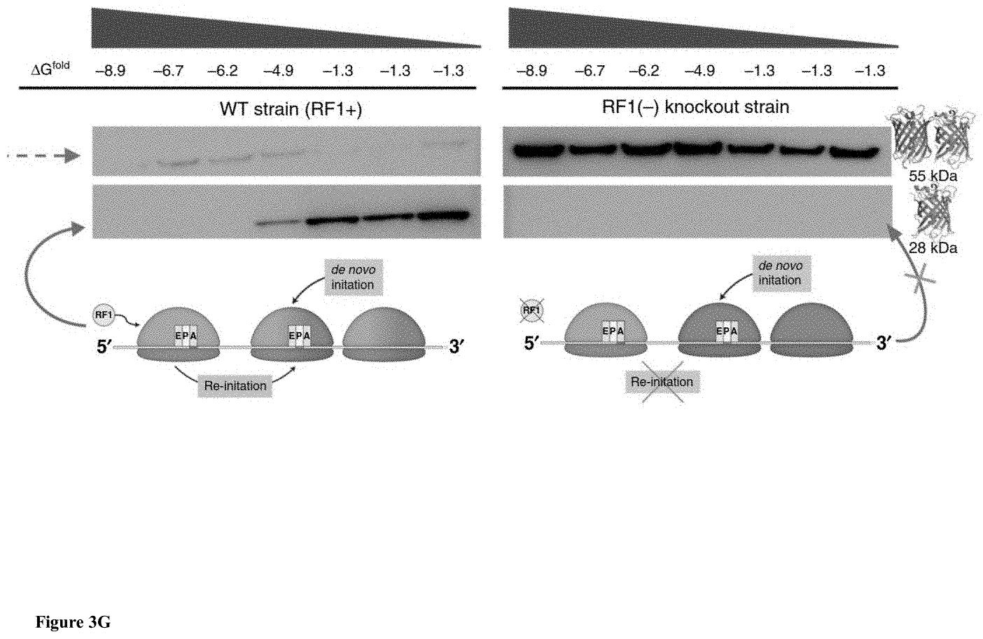

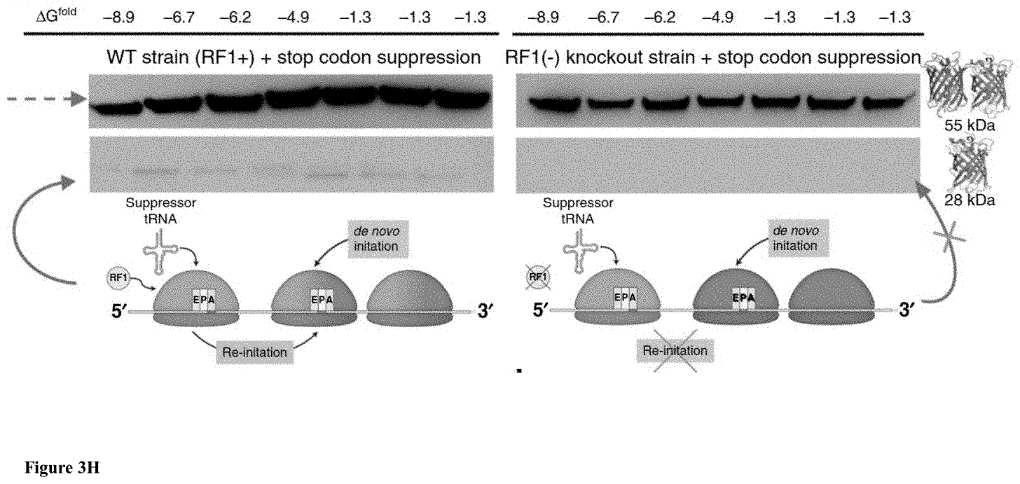

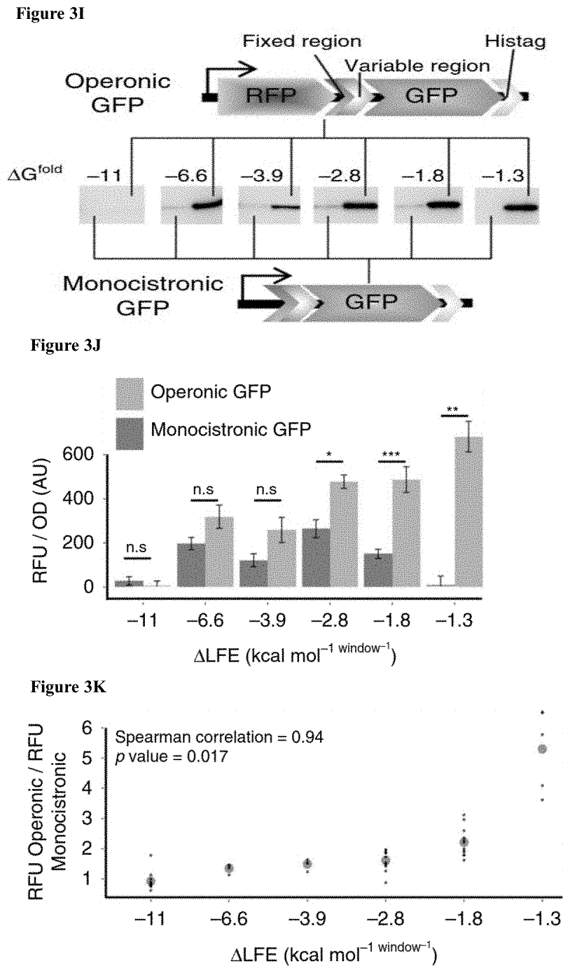

Strains and plasmids: The bacterial strains used in this study were Escherichia coli K-12 MG1655 and E. coli C321.ΔprfA EXP (Addgene #48998). For genetic code expansion, experimental strains were transformed with a pEVOL plasmid harboring the Methanosarcina mazei (Mm) orthogonal pair of Mm-PylRS/Mm-tRNA_{CUA}^{PrK} (Pyl-OTS). The dual reporter system plasmid was adapted from the pRXG plasmid, and the random sequence was inserted using random primer amplification followed by Gibson assembly. The expression of the synthetic operon was controlled by the Lac operator as to not affect bacterial fitness by the variability of the random sequence, which is only expressed when IPTG is added. To control for known stop codon context effects, the first six nucleotides in this variable region (ACUAGU) were fixed. After assembly, the library was transformed into E. coli DH5α, where library complexity was measured to be ˜10⁴ by counting colony-forming units. The library was then purified using a Miniprep kit [Promega] and transformed into the E. coli MG1655 and C321 strains mentioned above. All E. coli MG1655 clones were subjected to fluorescence-activated cell sorting (FACS) [FACSAria, BD Biosciences]. In addition, individual clones were isolated using agar plating, and their plasmids isolated and sequenced (Table 2 and 4). Each variable sequence that did not present an additional stop codon in the variable region was named pRXNG and given a running number name [i.e. pRXNG 60 is clone #60] and its RFP and GFP expression levels were measured. Deletion of the RFP gene for the experiments detailed in FIG. 3I-J was achieved by Gibson assembly using the following primers, forward: ATAACAATTTCACACAGAAACAGAAGCTGGTTCTGGCGAATAGACTAG (SEQ ID NO: 1), reverse: (TTCTGTTTCTGTGTGAAATTGTTATCCG (SEQ ID NO: 2).

Fluorescence-activated cell sorting (FACS): Bacterial cells were grown overnight induced with 1 mM IPTG, washed with PBS and sorted by using FACS [FACSAria, BD Biosciences]. The entire cell population was sorted into 8 bins based on constant mRFP1 fluorescence and varying Superfolder GFP (sfGFP) fluorescence, thereby normalizing sfGFP levels to those of mRFP1. Each bin accounted for ˜12.5% of the entire population, using an 85-micron nozzle at minimal flow. The 8 sorted bins were re-run to map sorting accuracy, which was found to be high (˜90% of cells were distributed within 3 bins around any selected bin). Controls consisted of bacterial cells that did not harbor the synthetic operon plasmid. Analysis was performed, and figures were created using FlowJo software. The gating strategy was as follows: The preliminary FSC-A/SSC-A gates were 630-17,000 and 60-3,000, respectively, the SSC-W/SSC-H gates were 0-110,000 and 450-45,000, respectively, and the FSC-W/FSC-H gates were 12,000-62,000 and 200-4,000, respectively. Cells that expressed RFP, which served as the positive and normalizing control with levels between 3,500-15,000, were further gated. Next, the resulting population (49.7% of the total population) was gated into 8 equal groups divided and defined by GFP expression. Each group was intended to represent ˜12.5% of the parent population.

Library construction, next-generation sequencing and data analysis: Isolated bacteria from each bin were transferred to LB media and grown for 8 h at 37° C. Cell were harvested and subjected to plasmid extraction using a Miniprep kit [Promega]. Library construction for Illumina MiSeq next-generation sequencing was done under the Illumina metagenomic protocol. In each bin, a 118 bp synthetic operon amplicon, which includes the variable region, was PCR-amplified. In two rounds of amplification, the Illumina primer sequence, unique hepta-nucleotide indexes and adaptors were added to each amplicon library. The libraries were then sequenced using the Illumina V2 (300 cycles) kit. The resulting sequencing data was processed and parsed with the DADA2 package for R. All identical sequence reads in each bin were aggregated, and the 10,000 most abundant sequences of each bin were obtained. In the eight bins, the minimal sequence depth was 2-10 reads. From the 10,000 sequences of each bin, all sequences which contained an additional stop codon in the variable region were removed and the remaining sequences were filtered to include only sequences with one of the three efficient start codons (ATG, GTG, TTG) in any in-frame position of the variable region. This process resulted in 2,580-2,694 unique sequences in each bin. The mean ΔG_{fold} and the 99% confidence interval were calculated for each bin (see computational method for calculation) and the statistical significance comparing each pair of consecutive bins was done using a two-tail Wilcoxon rank test.

RFP and GFP expression from the dual reporter with the random library: Measurements from triplicate bacterial growth cultures in a 96-well plate [Thermo Scientific] covered with Breathe-Easy seals [Diversified Biotech] were recorded overnight using a 37° C. incubated plate reader [Tecan]. RFP (excitation: 584 nm; emission: 607 nm) and GFP (excitation: 488 nm; emission: 507 nm) expression levels and OD₆₀₀ were measured every 15 minutes. The values presented the plateau value of each clone, which was measured in at least 5 experimental repeats (n>3). We reasoned a priori that normalizing fluorescence levels to OD was appropriate, as over-expression of the reporters between clones could have led to changes in total protein amounts among clones. Normalizing to OD, as a proxy for cell number per well, was more relevant for comparing GFP expression and for comparison between the Western blots and fluorescent measurement, which were also normalized to OD.

Western blots: Bacterial cultures were normalized to the same OD₆₀₀, after which 10 μL aliquots were mixed with 10 μL MOPS buffer and 5 μL SDS buffer and incubated for 10 min at 70° C. Samples were loaded onto a 4-20% SDS gel [Genscript] and transferred to a PVDF membrane [Bio-Rad] using an E-blot protein transfer apparatus [Genscript]. After transfer, anti-His tag antibodies were used to probe the transferred proteins. Antibody binding was visualized using an ImageQuant LAS 4000 imager [Fujifilm]. Densitometry analysis was performed using the gel tool in ImageJ V1.52a software.

Stop codon suppression by genetic code expansion: Genetic code expansion by stop codon suppression was introduced to suppress the UAG stop codon in E. coli MG1655, where the unnatural amino acid N-propargyl-1-lysine (1 mM final concentration in culture) was incorporated in response to the UAG stop codon at the end of the RFP gene using the Mm pyrrolysine tRNACUApyl and pyrrolysyl-tRNA synthetase orthogonal pair, expressed from the pEVOL plasmid. Induction of PylRS was performed by adding 0.5% L-arabinose [Sigma-Aldrich] to the growth medium.

Quantitative PCR: Quantitative PCR was performed according to MIQE guidelines. E. coli MG1655 cells were transformed with the pRXNG clones and grown to logarithmic phase (OD₆₀₀ of 0.4-0.5), harvested, and extracted with a GeneJET RNA purification kit [Thermo Scientific] for total RNA extraction, yielding 50 μL of RNA with a concentration of ˜400 ng μL⁻¹ and of high purify (A₂₆₀/A₂₈₀=2.1). This step was followed by DNase (RNase free) [Thermo Scientific] digestion using the kit protocol and guidelines. RNA was immediately reverse-transcribed into cDNA with an iScript cDNA Synthesis kit [Biorad], under kit guidelines with 1 μg RNA. Real-time PCR was performed using a KAPA SYBR FAST qPCR reagent [Sigma] in a CFX qPCR instrument [Bio Rad], with duplicates of 10 μL reactions containing 1.2 μL of cDNA in each well of a qPCR 384 well-plate [Bio Rad]. The thermocycler parameters were set to 94° C. for 2 min, 40 cycles of 94° C. for 15 sec, 59° C. for 25 sec, and 72° C. 30 sec. Two synthetic operon sample amplicons were targeted: 1) an RFP target, upstream of the variable region, between positions 394-528 with a length of 135 bases; forward primer: GACGGTCCGGTTATGCAGAA (SEQ ID NO: 3), reverse primer: TTCAGCGTCGTAGTGACCAC (SEQ ID NO: 4); 2) a GFP target, downstream of the variable region, between positions 873-1008 with a length of 136 bases; forward primer: CAAGCTCCCAGTACCATGGC (SEQ ID NO: 5), reverse primer: GCGCTCTTGTACATAGCCCT (SEQ ID NO: 6). In addition, a normalizing gene (16S rRNA) was used with primers 1369F-CGGTGAATACGTTCYCGG (SEQ ID NO: 7) and 1492R-GGTTACCTTGTTACGACTT (SEQ ID NO: 8). Both melt curves and agarose gel electrophoresis were used to confirm primer specificity. For all primers, only one amplicon of the correct size was detected. Sample primer pair calibration curves presented r² values of 0.991 and 0.998 for primers 1 and 2, respectively, with a dynamic range between Cq 3 and 18, while the LOD was Cq 14.18. The normalizing gene primer calibration curve presented an r² value of 0.996 with a dynamic range between Cq 15 and Cq 23, while the LOD was Cq 14.56. Data analysis was manually performed using Bio-Rad CFX Manager V3.1 software.

Protein purification and mass spectrometry analysis: Proteins were fused to a 6×His tag and purified by nickel resin affinity chromatography. Purified protein samples were analyzed by LC-MS [Finnigan Surveyor/LCQ Fleet, Thermo Scientific].

Calculation of ΔG_{fold} for synthetic operon clones: All calculations were made using the Vienna package (default settings), with the extracted mRNA sequence window upon which the ΔG_{fold} calculation was made for each clone obeying the two following constraints: First, the start of the window was +9 nucleotides from the first nucleotide of the UAG stop codon. This was done to simulate mRNA secondary structure which exists outside the ribosomal entry tunnel. Second, the window size used was experimentally determined, with a threshold requirement, namely correlation between ΔG_{fold} and GFP expression should be robust using window sizes ranging from 30 to 50 nts (length of the random region of interest =24 nt). Optimal correlation was found with a window size of 37 nt. As such, this window size was used for the results presented.

Simulation of theoretical ΔG_{fold} of random library clones. Each set of 10⁶ random sequences was sampled from a population of uniform nucleotide distribution and filtered as follows. i) 37nt sample: Include random sequences of length 37nt containing in-frame one of the start codons (AUG, GUG, UUG) and not containing one of the stop codons (UGA, UAG, UAA). ii) 24+13 sample: this sample is mimicking the sequences of the random library used herein. It includes random sequences of length 24nt containing in-frame one of the start codons (AUG, GUG, UUG) and not containing one of the stop codons (UGA, UAG, UAA), and concatenated with the suffix [AAGGGCGAGGAGC] (giving a total length of 37nt). iii) Unconstrained sample: Include random sequences of length 37nt.

Species selection: Species were chosen for taxonomic diversity and overlap with public datasets (N=183), with emphasis on bacteria (N=128) and archaea (N=49). Genomic sequences and annotations were obtained from the Ensembl database.

ΔLFE (folding bias) calculations: To estimate the tendency of short-range interactions within the mRNA strand to form stable secondary structures (i.e., Local Fold Energy [LFE]), sequences were broken into 40 nt-long windows and the minimum folding energy was calculated using RNAfold from the Vienna package (using default settings). To identify regions where strong or weak secondary structure may be functional, rather than a side effect of selection acting on amino acid sequence, or nucleotide or codon composition (see Randomization, below), the influence of these factors was controlled by comparing LFE of the native sequence to a set of randomized sequences maintaining these factors. The difference between the LFE of the native and randomized sequences is denoted as ΔLFE or local folding bias. If only the amino acid sequence, nucleotide composition, and codon composition are under selection at a given position, one expects ΔLFE to be close to 0. Any statistically significant deviation from this value indicates that additional factors maintained under selection are needed to explain the measured native LFE value.

Since this study focused on mRNA, only those regions surrounding protein-coding genes are included; genes shorter than 40 nt were excluded. Genes with a length that is not a multiple of 3, those containing an internal stop codon or where the last codon is not a stop codon were also excluded. To identify features related to translation termination, ΔLFE for all included genes from a given species was averaged at each position, relative to the stop codon.

Randomization: The randomized sequences were sampled from the distribution representing the null hypothesis, namely that only the amino acid sequence, and nucleotide and codon composition (see below) are under selection at a given position in the coding sequence, and only the nucleotide composition is under selection in a given UTR. To produce random sequences maintaining these properties, synonymous codons within each coding sequence were randomly permutated, and the nucleotides of each UTR were randomly permutated. Regions overlapping multiple coding sequences were maintained without permutations. Codons containing one or more ambiguous nucleotides (‘N’ bases) were likewise maintained without permutations. Synonymous codons were identified according to the gene translation table for each species. Randomization of the non-coding UTR regions were randomized by permutating only the nucleotide composition.

RTS model: To estimate the number of genes within each species likely to present an RTS after its stop codon, each gene in all species were examined. The RTS was defined and deemed present if three conditions were met: 1. The gene is separated from its successor by an annotated intergenic region of 25 nucleotides or more, or the next gene is on the opposite DNA strand; 2. At least five consecutive windows opening in the range of −10 to +20 nucleotides (meaning that the windows cover the region of between the −10 to +59 nucleotides, as the window size is 40, relative to the end of the stop codon), and that the ΔLFE is negative; and 3. A threshold of ΔG_{fold}<−6 kcal mol⁻¹ window⁻¹ must be crossed in at least one of the five or more negative ΔLFE windows. If all conditions are met, the longest consecutive stretch of windows (5 or more) would be defined as a putative RTS, and the gene will be counted as being followed by an RTS. By repeating this process for all annotated genes of a given species, the fraction of genes followed by an RTS can be calculated. All parameter values used to define an RTS in this model are preliminary, but the parameter sensitivity of the model is low, and the results are robust in large parameter space.

Plotting: Distributions of multiple genes or averages for multiple species are presented using the statistics commonly used for boxplots, as follows. The shaded region spans the 25th and 75th percentiles, with the median plotted as a darker line. Elements outside this region are presented by their density (blue shading in the background). Densities are shown as kernel density estimates (KDEs), computed separately at each position, using a Gaussian kernel with a bandwidth of 0.5. Plots were created using Scikit Learn and Matplotlib. Taxonomic trees are based on NCBI taxonomy and were plotted using the ete toolkit.

Statistical analysis: All statistical analysis was performed under the guidelines of the tests described in-text. The minimal p-value noted in the text was selected to be 10⁻³⁰. In all cases where the precise p-value calculated was smaller (i.e., more significant), the test-statistic score is given. To test whether ΔLFE values for a one-sample group of genes are statistically different, as compared to a reference value (e.g., for the RTS model), the Wilcoxon signed-ranks test was used on the ΔLFE (randomized AG-native AG) values for all genes (20 randomization repetitions for each gene). To test whether ΔLFE values for two-sample groups of genes are statistically different from each other, the Mann-Whitney U test was used on the ΔLFE (randomized AG-native AG) values for all genes (with 20 randomization repetitions for each gene). As such, the test N was 20 times the number of data points of the original sample. The p-values and test statistics are reported for the position of the most extreme test-statistic, whereas the surrounding regions showed consistent and significant results.

Additional data sources: Experimentally determined operonic positions were obtained from ODB4. Protein-abundance data was obtained from PaxDb. Experimentally determined 3′-UTR lengths were obtained from regulondb. Termination type data for E. coli genes were obtained from WebGesTer.

TABLE 3

| | | SEQ |
|---|---|---|
| | | ID |
| Clone | Sequence | NO: |
| 29 | TAGACTAGTTTACTTCCCTCCTCTATTCTATCAAA | 9 |
| | GGGCGAGGAGCTCTTTACTGGCGTAGTACCAATT | |
| 33 | TAGACTAGTGGCCCGTCAACTTGTGTGGTTTATAA | 10 |
| | GGGCGAGGAGCTCTTTACTGGCGTAGTACCAATT | |
| 52 | TAGACTAGTTGGGAGATGAATTTAAACCGGAACAA | 11 |
| | GGGCGAGGAGCTCTTTACTGGCGTAGTACCAATT | |
| 56 | TAGACTAGTCCAACACTGGTGTTTCGCGGATGGAA | 12 |
| | GGGCGAGGAGCTCTTTACTGGCGTAGTACCAATT | |
| 57 | TAGACTAGTTCCCCTGAACCTATATTGCTTGCTAA | 13 |
| | GGGCGAGGAGCTCTTTACTGGCGTAGTACCAATT | |
| 62 | TAGACTAGTCTAACTGTACAACTCTTACTGTCGAA | 14 |
| | GGGCGAGGAGCTCTTTACTGGCGTAGTACCAATT | |
| 71 | TAGACTAGTCAAATTGTTTTGGATCGGAGGAGGAA | 15 |
| | GGGCGAGGAGCTCTTTACTGGCGTAGTACCAATT | |
| 91 | TAGACTAGTGGTTTTAGGGCGGATCAATTGTTAAA | 16 |
| | GGGCGAGGAGCTCTTTACTGGCGTAGTACCAATT | |
| 96 | TAGACTAGTCGGGGAAAAAGGGCGGTGCGATGTAA | 17 |
| | GGGCGAGGAGCTCTTTACTGGCGTAGTACCAATT | |
| 101 | TAGACTAGTATCCGTATATTGTTATTGGTCCTGAA | 18 |
| | GGGCGAGGAGCTCTTTACTGGCGTAGTACCAATT | |
| 110 | TAGACTAGTGGCGCGCCTCTTAATATGGGTCGTAA | 19 |
| | GGGCGAGGAGCTCTTTACTGGCGTAGTACCAATT | |
| 111 | TAGACTAGTGCGTCTATTCCGCCGCCCAGCCGTAA | 20 |
| | GGGCGAGGAGCTCTTTACTGGCGTAGTACCAATT | |
| 202 | TAGACTAGTCCAGTGGCTTCAAGCTCACTGCCTAA | 21 |
| | GGGCGAGGAGCTCTTTACTGGCGTAGTACCAATT | |
| 203 | TAGACTAGTGTATGTGAAGCCTTGGCGACGTATAA | 22 |
| | GGGCGAGGAGCTCTTTACTGGCGTAGTACCAATT | |
| 207 | TAGACTAGTATGATTTCTACAGTCAAAAGGGATAA | 23 |
| | GGGCGAGGAGCTCTTTACTGGCGTAGTACCAATT | |
| 208 | TAGACTAGTGGCAGACACTGTATGTATATATTGAA | 24 |
| | GGGCSAGGAGCTCTTTACTGGCGTASWACCAATT | |
| 209 | TAGACTAGTGAGGCACTGATAATGTGTTTGGACAA | 25 |
| | GGGCGAGGAGCTCTTTACTGGCGTAGTACCAATT | |
| 212 | TAGACTAGTCGTAAACGAATGATGTCGTGGCGTAA | 26 |
| | GGGCGAGGAGCTCTTTACTGGCGTAGTACCAATT | |
| 214 | TAGACTAGTATGTTGTGTTCAAACGAAATCCAGAA | 27 |
| | GGGCGAGGAGCTCTTTACTGGCGTAGTACCAATT | |
| 216 | TAGACTAGTAAAAAAATGTGGCGGCAAAATGGAAA | 28 |
| | GGGCGAGGAGCTCTTTACTGGCGTAGTACCAATT | |
| 220 | TAGACTAGTTGGGTATCAATGGCAATTTCTCTTAA | 29 |
| | GGGCGAGGAGCTCTTTACTGGCGTAGTACCAATT | |
| 222 | TAGACTAGTATGGCTAGGTTAATGGCTGGCAACAA | 30 |
| | GGGCGAGGAGCTCTTTACTGGCGTAGTACCAATT | |
| 225 | TAGACTAGTTTGCTTTCGTTCAATTTAAACTATAA | 31 |
| | GGGCGAGGAGCTCTTTACTGGCGTAGTACCAATT | |
| 226 | TAGACTAGTTGGCCCTTGATTTCACCTATGTTAAA | 32 |
| | GGGCGAGGAGCTYTTTACTGGCGTAGTACCAATT | |
| 230 | TAGACTAGTCGGTCGATTAGTTGGATGTATGCTAA | 33 |
| | GGGGCGAGGAGCTCTTTACTGCGTAGTACCAATT | |
| 232 | TAGACTAGTGTAAATTTAATGAGTTCTCGTGAGAA | 34 |
| | GGGCGAGGAGCTCTTTACTGGCGTAGTACCAATT | |
| 233 | TAGACTAGTTCAGCACATTTAGGTGTGCCGTACAA | 35 |
| | GGGCGAGGAGCTCTTTACTGGCGTAGTACCAATT | |
| 235 | TAGACTAGTTCTCACCTGGAACCGAATAATGGGAA | 36 |
| | GGGCGAGGAGCTCTTTACTGGCGTAGTACCAATT | |
| 236 | TAGACTAGTTTGCTTTGGTGTGCGAAGGTCCCGAA | 37 |
| | GGGCGAGGAGCTCTTTACTGGCGTAGTACCAATT | |
| 238 | TAGACTAGTCCCGTGCCATGTAGAAAGAATCAGAA | 38 |
| | GGGCGAGGAGCTCTTTACTGGCGTAGTACCAATT | |
| 244 | TAGACTAGTAAGATGAACCTAAAAATGTCTCCAAA | 39 |
| | GGGCGAGGAGCTCTTTACTGGCGTAGTACCAATT | |
| 245 | TAGACTAGTGAGGCACTGCGAATGTGTTTGAACAA | 40 |
| | GGGCGAGGAGCTCTTTACTGGCGTAGTACCAATT | |
| 249 | TAGACTAGTGGCAGACACTGTATGTATATATTGAA | 41 |
| | GGGCGAGGAGCTCTTTACTGGCGTAGTACCWATT | |

Synthetic Operon Sequence: The RFP stop codon is followed by the fixed 6-nucleotides and the 24-nucleotides random sequence, which vary between clones. The sequence used for the synthetic operon is provide in SEQ ID NO: 42.

Monocistronic GFP Sequence (ΔRFP): The Lac operator, 18 bases from the RFP gene that were left-in, followed by the fixed 6-nucleotides and the 24-nucleotides random sequence, which vary between clones. The sequence of the monocistronic GFP is provided in SEQ ID NO: 43.

### Example 1: mRNA Structure Drives Distal Gene Expression in a Synthetic Operon

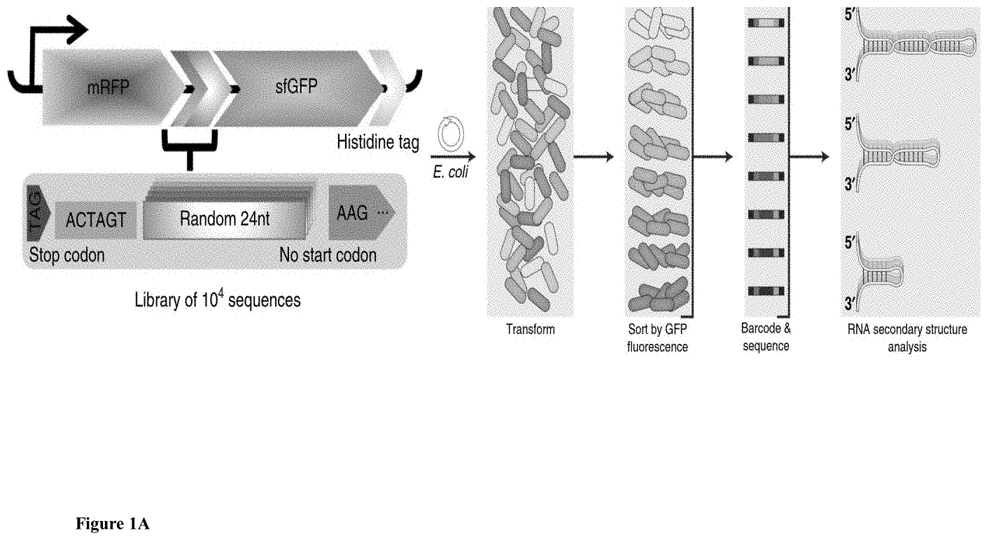

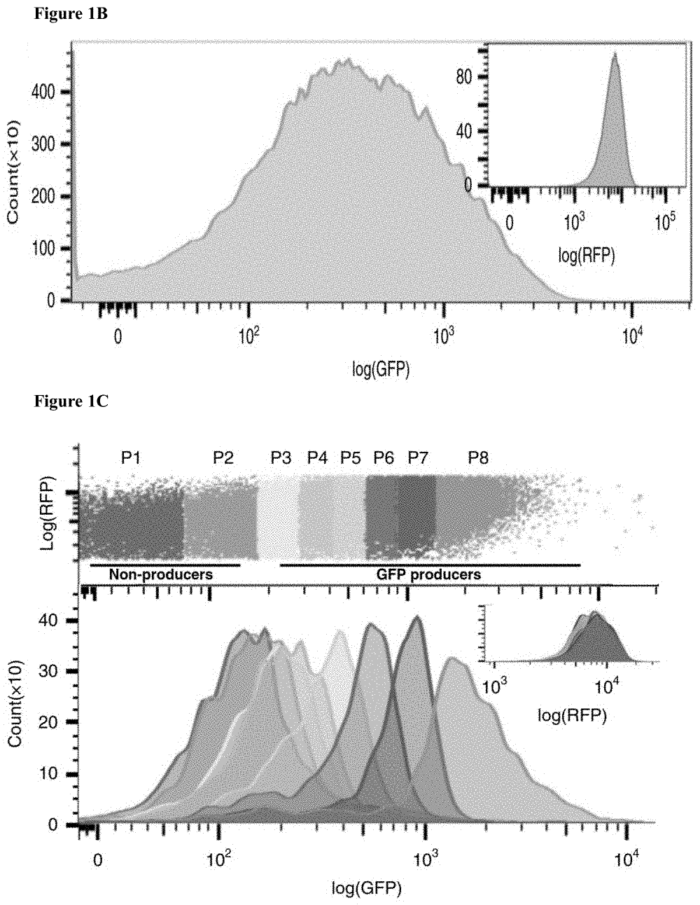

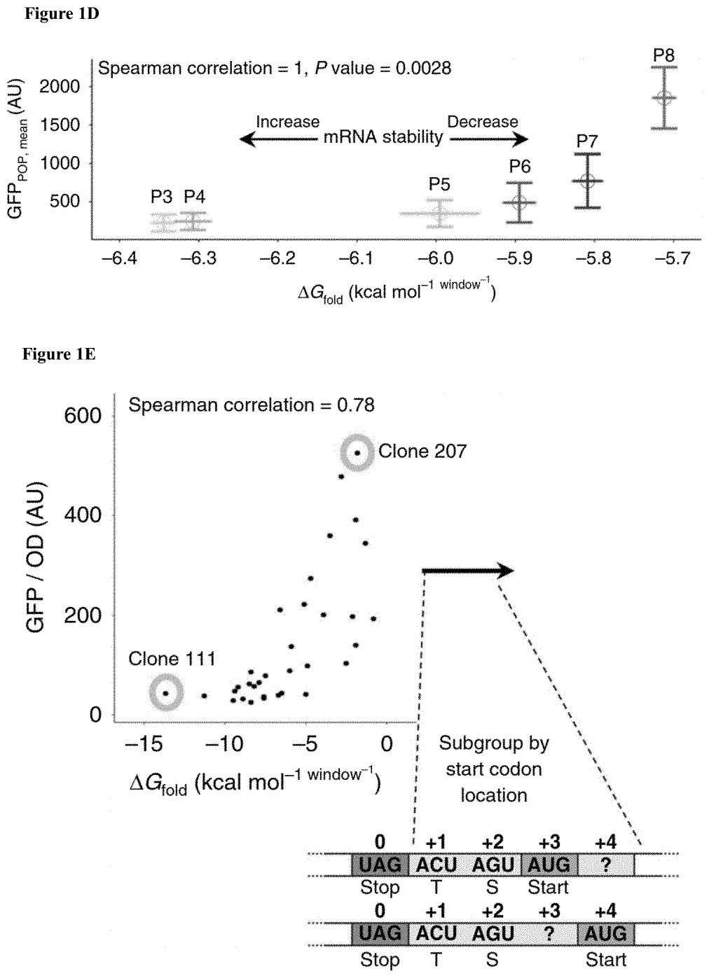

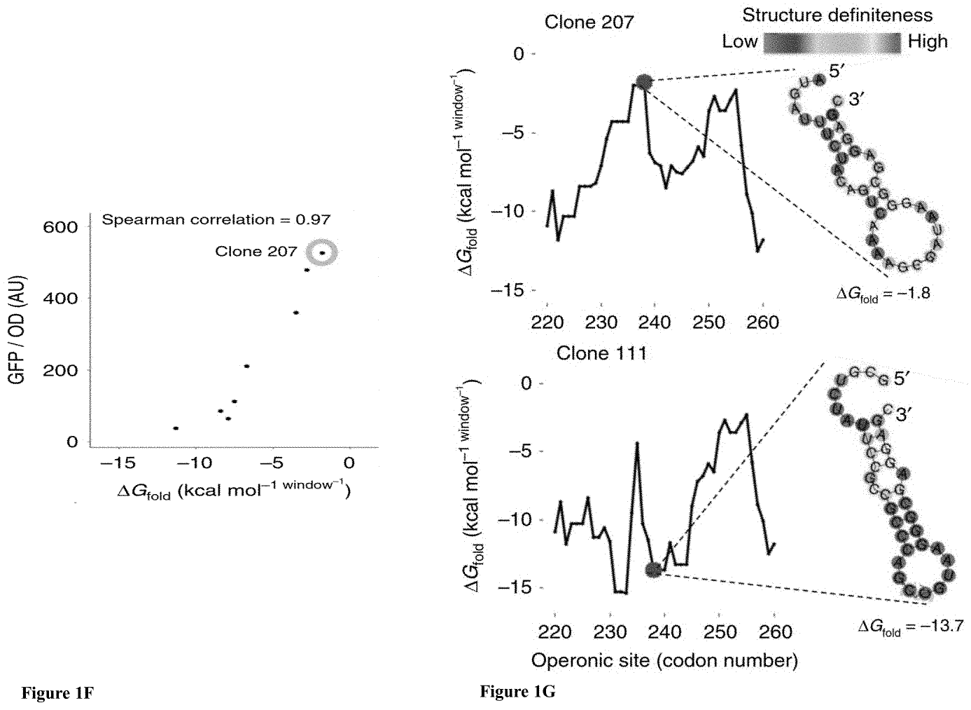

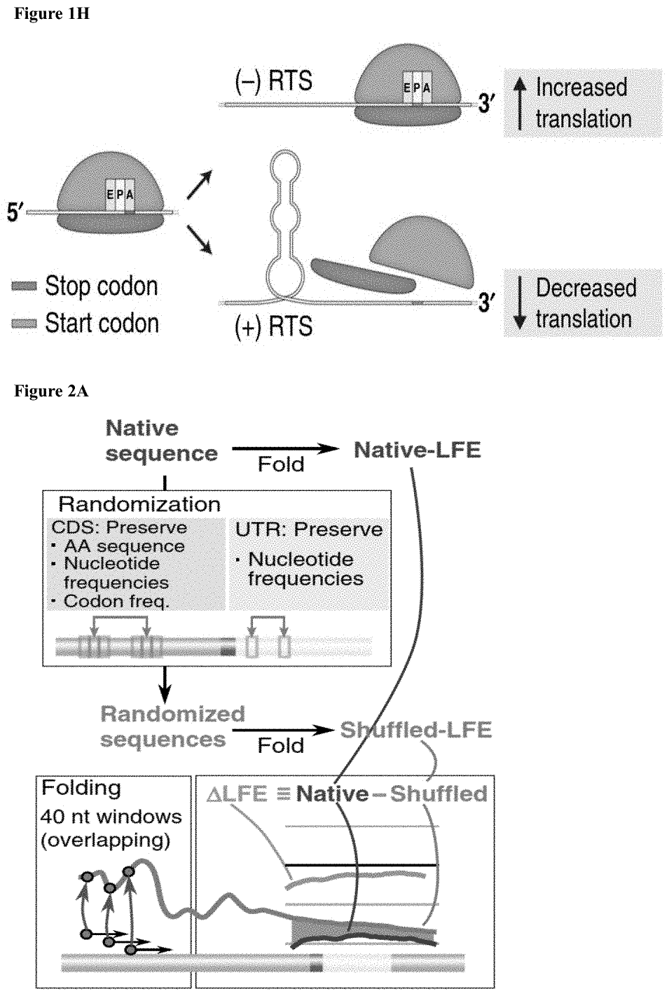

To test the relation between mRNA secondary structure and translation re-initiation, a library of operons based on the pRXG plasmid was assembled (FIG. 1A). These synthetic operons comprise a proximal gene encoding red fluorescent protein (RFP) and a distal gene encoding polyhistidine-tagged green fluorescent protein (GFP), separated by a stretch of 24 random nucleotides in the inter-cistronic region, downstream of the RFP stop codon. The library was transformed into Escherichia coli MG1655 cells and sorted according to GFP expression levels into eight binds spanning three orders of magnitude (FIG. 1B), using flow cytometry (FIG. 1C). Each bin was barcoded, sequenced, and the weighted Gibbs free energy average (ΔG_{fold}) of mRNA secondary structure in the variable sequence region in that bin was calculated.

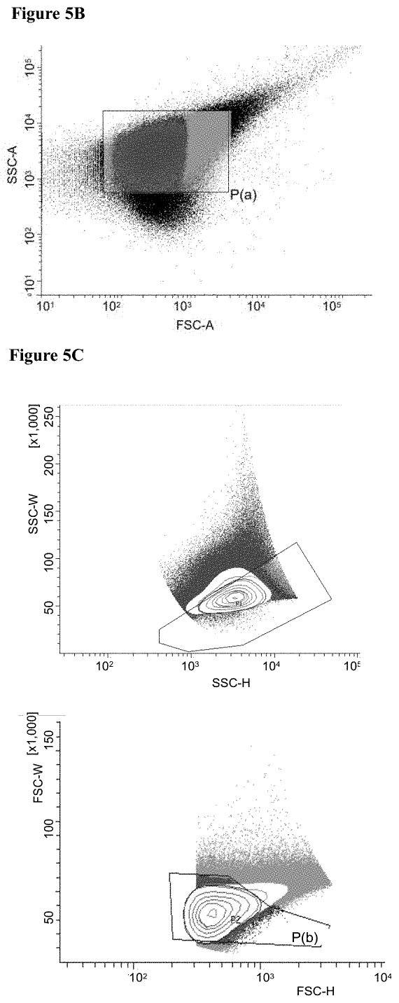

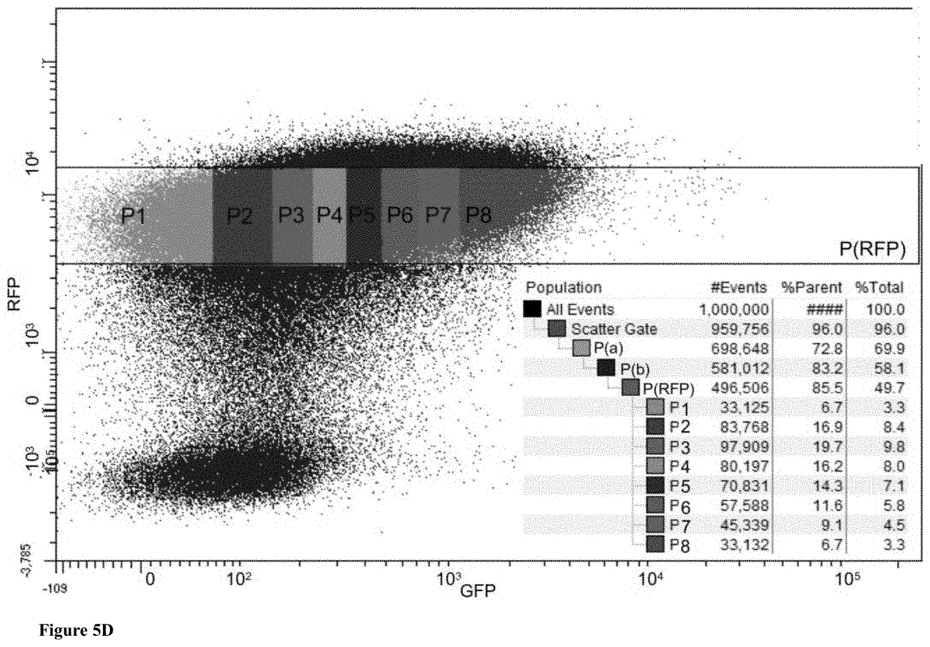

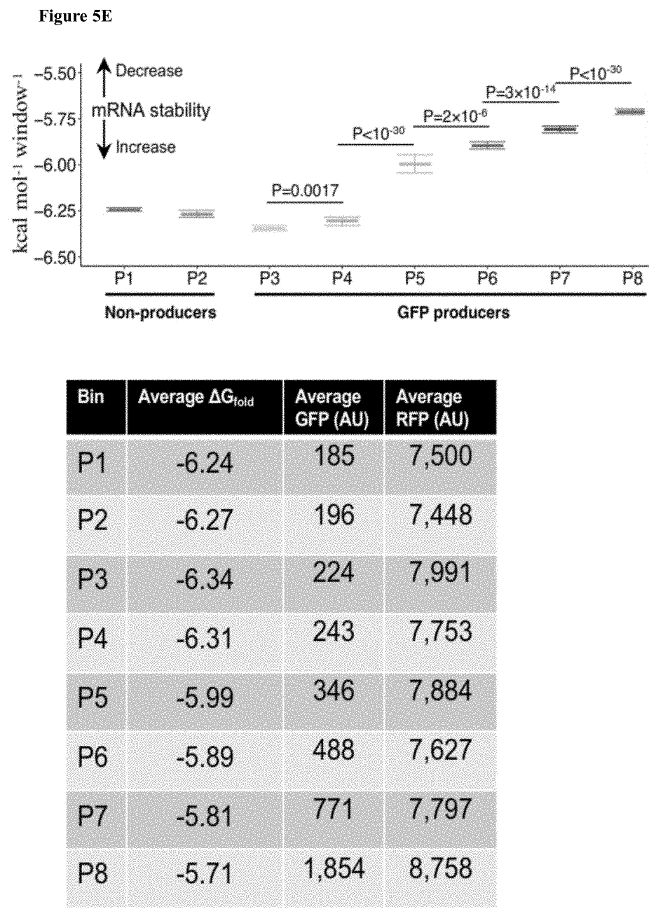

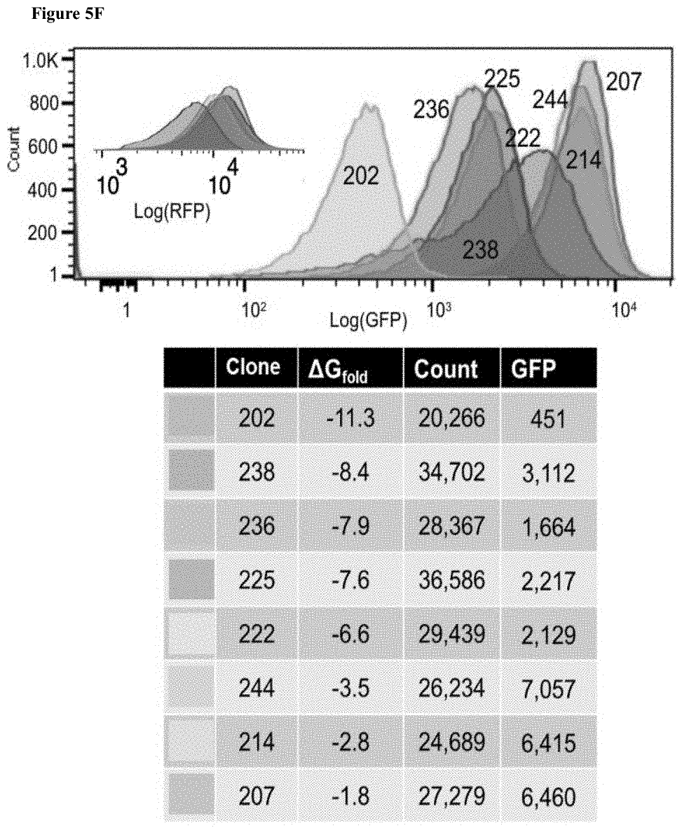

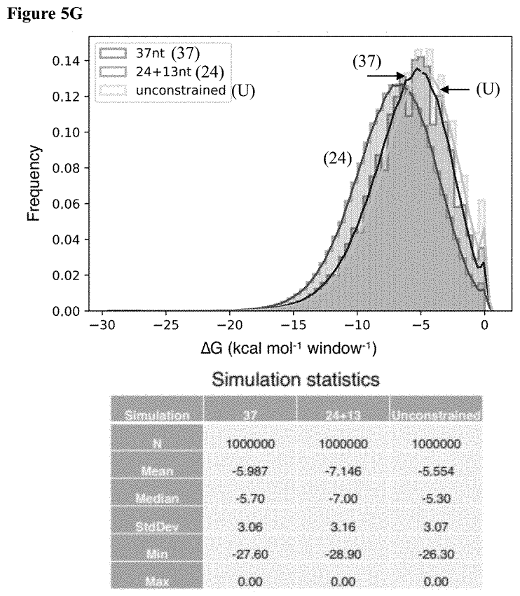

The first two bins (P1 and P2) exhibited GFP expression levels that were not higher than those in the negative wild-type bacteria controls (FIGS. 5A-G). As such, bins P1 and P2 were labeled as non-producing populations and not further analyzed. The results from the other bins (P3-P8), however, revealed significant correlation between observed GFP levels and the calculated mean ΔG_{fold} of the ˜3×10³ unique sequences in each bin (Spearman correlation p=1, n=6, p-value=0.0028) (FIG. 1D). These results illustrate the inverse correlation between expression levels of the distal gene-encoded GFP and mRNA folding stability, such that sequences with lower stability in the variable region were significantly enriched in high GFP-producing populations, and vice versa (FIG. 5E).

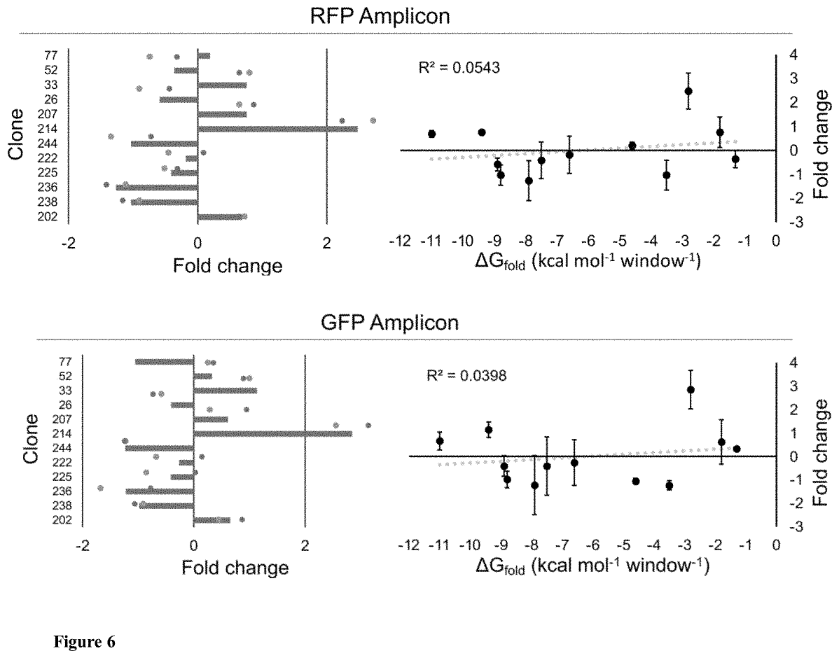

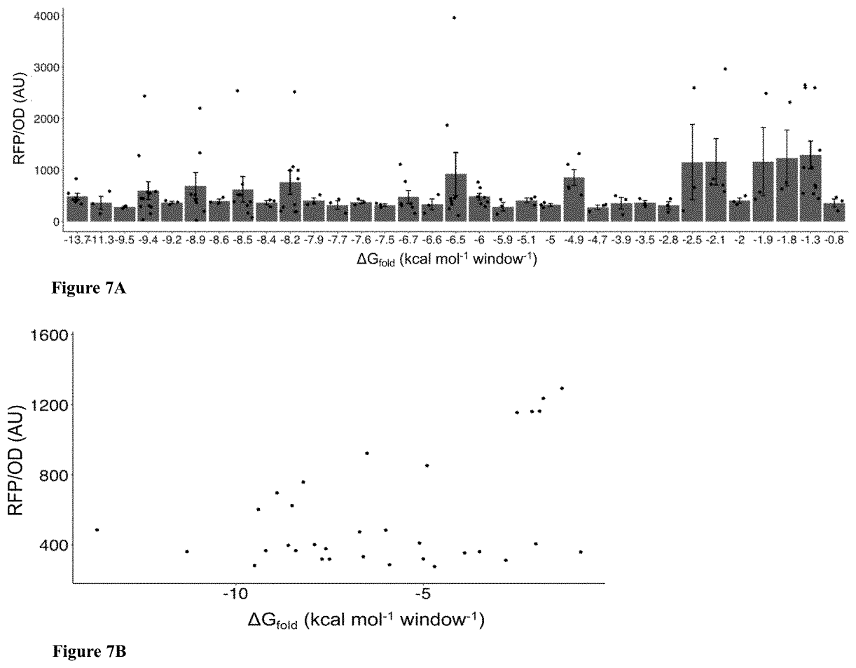

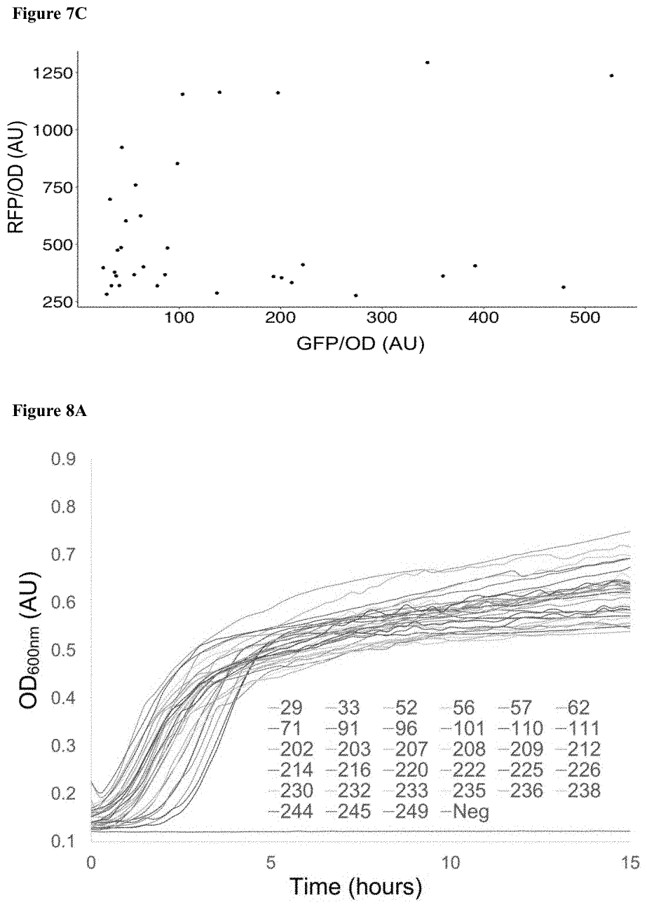

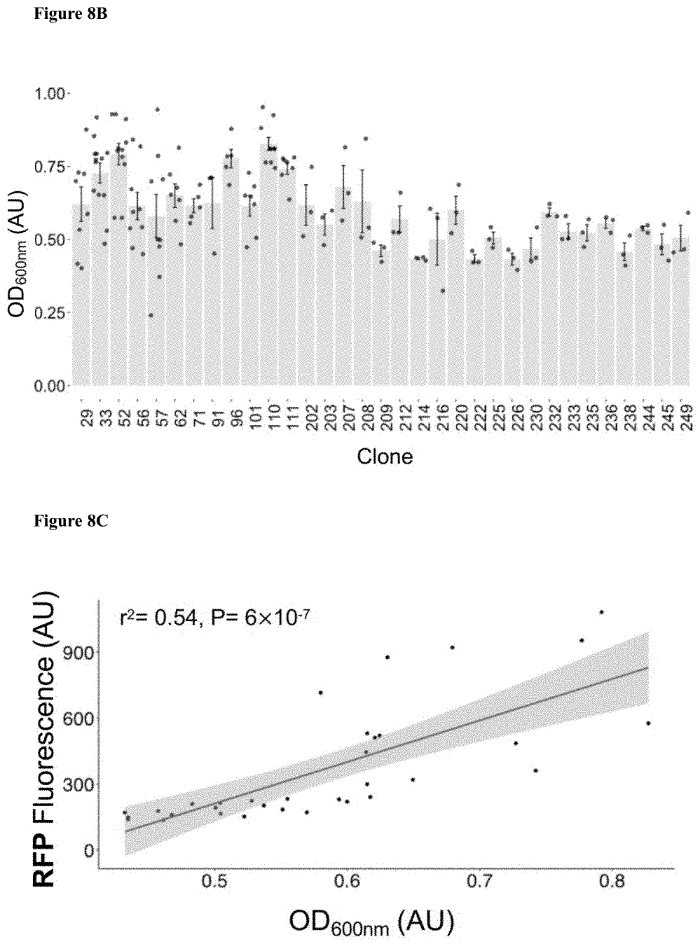

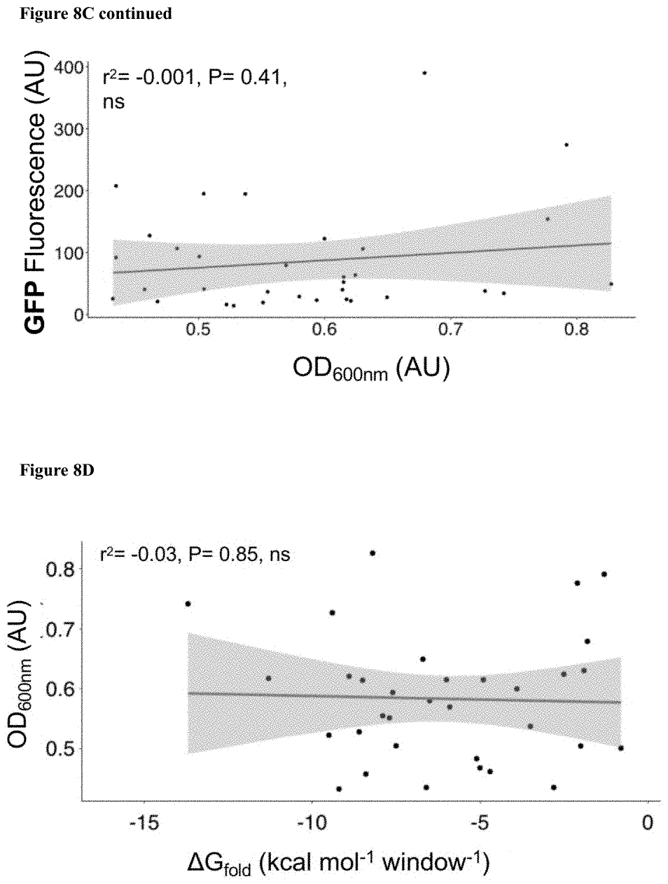

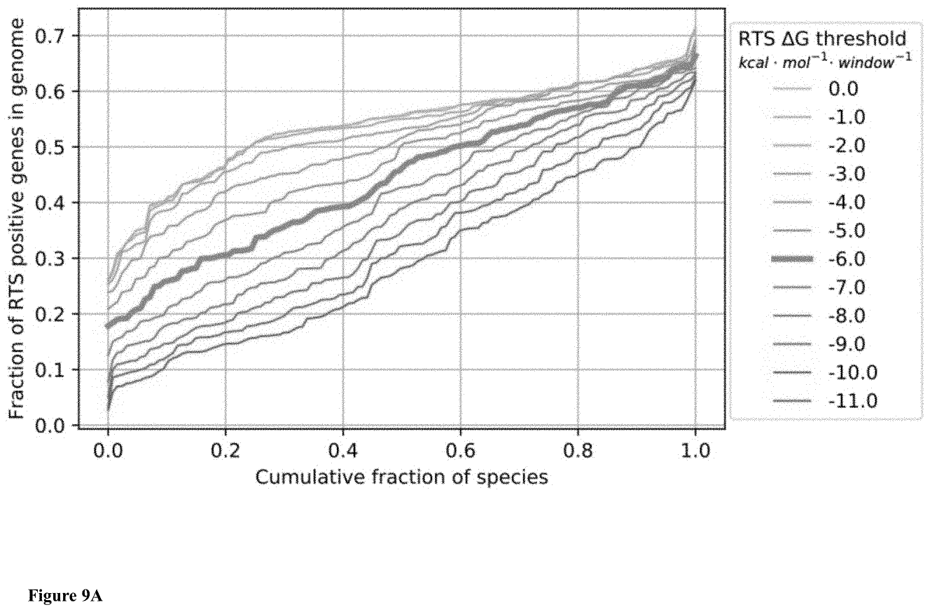

Next, individual clones from each bin were sorted and sequenced. Thirty-three clones in which the variable inter-cistronic sequence encodes at least one of the six most abundant start codons for translation initiation also lacked additional in-frame stop codons and presented a unique ΔG_{fold}. These clones were isolated, and their GFP expression levels were quantified (Table 1). Upon assessing the relation between ΔG_{fold} of the variable sequence and GFP expression, clear correlation was revealed (Spearman correlation ρ=−0.78, n=33, p-value<10⁻⁷) (FIG. 1E). Such correlation was independent of mRNA abundance (FIG. 6), expression of the upstream RFP gene (FIG. 7A-C), or of the location or identity of the start codon and adjacent Shine-Dalgarno (SD) sequence in the downstream GFP gene to which the ribosome binds (Table 2). No significant effect on growth rate was observed among the clones. Rather, the character of the clone-specific intergenic sequence had a significant impact on GFP levels but not on growth (FIG. 8A-D).

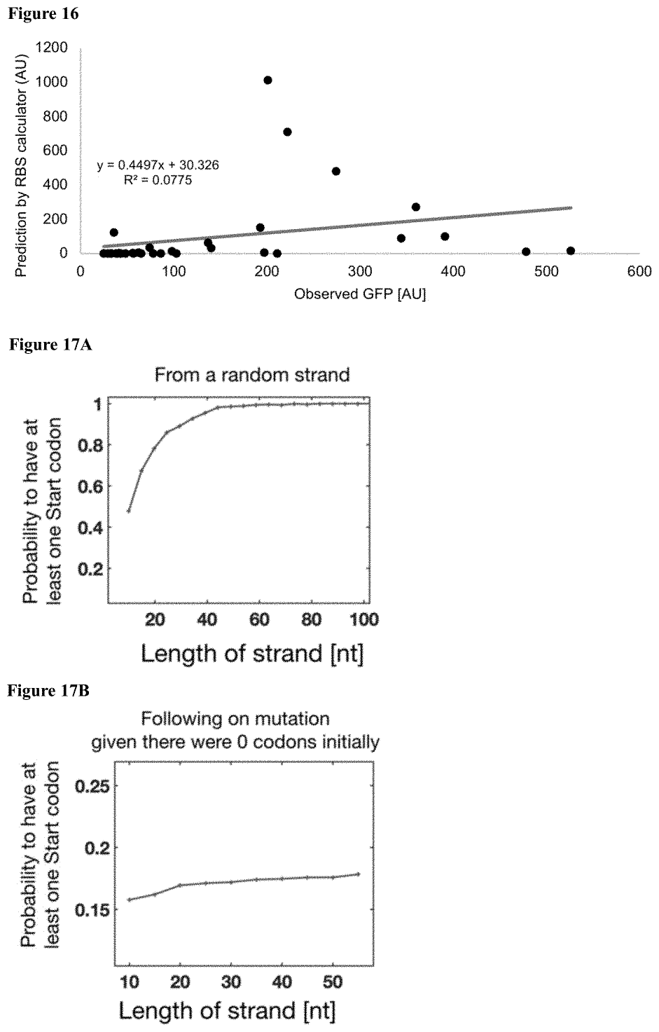

In a distinct subset of eight clones where variability in the start codon was further limited to only one of the three most used GFP-start codons (AUG, GUG, UUG), and variability in their position was limited to only three or four codons downstream of the RFP stop codon, the correlation was strengthened (Spearman correlation ρ=−0.98, n=8, p-val=4×10⁴) (FIG. 1F). In this subset, in which the SD sequence was identical for all clones, the GFP expression trend was confirmed at the population level using fluorescence-activated cell sorting (FACS) analysis (FIG. 5E). The results thus showed that distal operonic GFP gene expression is negatively affected by a stable mRNA secondary structure in the region directly downstream of the stop codon of the preceding gene (FIG. 1G). This structure was termed the ‘Ribosome Termination Structure (RTS), with the likelihood of RTS presence and its strength being defined by the magnitude of ΔG_{fold} (FIG. 1H). Correlation between observed GFP levels and those predicted upon de novo initiation using the RBS calculator based on the data in Table 2 in provided in FIG. 16.

TABLE 1
Characterization of individual clones
sequenced from the random library.
All sequences are available in Table 3.

| | | | | | | Start | | |
|---|---|---|---|---|---|---|---|---|
| | | | | | | codon | | |
| | | Avr. | Avr. | | Best | position | | |
| | | Fluor. | Fluor. | Avr. | re- | from | | MS |
| | | RFP/OD | GFP/OD | RFP/ | start | stop | Codon | Verifi- |
| Clone | ΔG_{fold} | [AU] ± SE | [AU] ± SE | GFP | codon | codon | rank | cation |
| 29 | −8.9 | 697 ± | 32 ± | 18.5 | I (AUU) | +8 | 6^{th} | |
| | | 254 | 4 | | | | | |
| 33 | −9.4 | 602 ± | 48 ± | 11.7 | V (GUG) | +8 | 2^{nd} | |
| | | 174 | 5 | | | | | |
| 52 | −1.3 | 1293 ± | 344 ± | 3.8 | M (AUG) | +5 | 1^{st} | |
| | | 269 | 12 | | | | | |
| 56 | −8.5 | 624 ± | 62 ± | 9.1 | V (GUG) | +6 | 2^{nd} | Yes |
| | | 248 | 4 | | | | | |
| 57 | −6.5 | 923 ± | 43 ± | 19.1 | L (UUG) | +8 | 3^{rd} | |
| | | 416 | 7 | | | | | |
| 62 | −6.7 | 474 ± | 39 ± | 12.5 | L (CUG) | +9 | 4^{th} | |
| | | 94 | 4 | | | | | |
| 71 | −4.9 | 853 ± | 98 ± | 8.8 | L (UUG) | +6 | 3^{rd} | |
| | | 153 | 4 | | | | | |
| 91 | −2.5 | 1155 ± | 103 ± | 13.4 | L (UUG) | +9 | 3^{rd} | Yes |
| | | 732 | 4 | | | | | |
| 96 | −2.1 | 1161 ± | 197 ± | 5.8 | V (GUG) | +8 | 2^{nd} | Yes |
| | | 452 | 7 | | | | | |
| 101 | −6.0 | 496 ± | 74 ± | 6.7 | L (UUG) | +6 | 3^{rd} | Yes |
| | | 64 | 24 | | | | | |
| 110 | −8.2 | 759 ± | 57 ± | 14.8 | M (AUG) | +8 | 1^{st} | |
| | | 228 | 4 | | | | | |
| 111 | −13.7 | 486 ± | 43 ± | 10.8 | I (AUU) | +5 | 6^{th} | Yes |
| | | 62 | 3 | | | | | |
| 202 | −11.3 | 362 ± | 38 ± | 9.0 | V (GUG) | +4 | 2^{nd} | |
| | | 126 | 2 | | | | | |
| 203 | −7.6 | 320 ± | 33 ± | 9.4 | L (UUG) | +7 | 3^{rd} | |
| | | 82 | 6 | | | | | |
| 207 | −1.8 | 1236 ± | 526 ± | 2.1 | M (AUG) | +3 | 1^{st} | |
| | | 541 | 53.5 | | | | | |
| 208 | −1.9 | 1163 ± | 140 ± | 6.5 | M (AUG) | +7 | 1^{st} | |
| | | 664 | 19 | | | | | |
| 209 | −4.7 | 276 ± | 274 ± | 1.1 | M (AUG) | +7 | 1^{st} | |
| | | 42 | 8 | | | | | |
| 212 | −5.9 | 287 ± | 137 ± | 2.0 | M (AUG) | +3 | 1^{st} | |
| | | 83 | 5 | | | | | |
| 214 | −2.8 | 313 ± | 478 ± | 0.7 | M (AUG) | +3 | 1^{st} | |
| | | 75 | 17 | | | | | |
| 216 | −0.8 | 360 ± | 193 ± | 1.9 | M (AUG) | +5 | 1^{st} | |
| | | 78 | 17 | | | | | |
| 220 | −3.9 | 354 ± | 201 ± | 1.7 | M (AUG) | +6 | 1^{st} | |
| | | 112 | 16 | | | | | |
| 222 | −6.6 | 333 ± | 211 ± | 1.7 | M (AUG) | +3 | 1^{st} | |
| | | 104 | 13 | | | | | |
| 225 | −7.5 | 319 ± | 78 ± | 4.0 | L (UUG) | +3 | 3^{rd} | |
| | | 23 | 4 | | | | | |
| 226 | −9.2 | 367 ± | 56 ± | 6.8 | L (UUG) | +5 | 3^{rd} | |
| | | 24 | 6 | | | | | |
| | | | | | M (AUG) | +9 | 1^{st} | |
| 230 | −5.0 | 320 ± | 41 ± | 8.2 | M (AUG) | +8 | 1^{st} | |
| | | 29 | 6 | | | | | |
| 232 | −7.6 | 378 ± | 36 ± | 10.3 | M (AUG) | +6 | 1^{st} | |
| | | 34 | 5 | | | | | |
| 233 | −8.4 | 398 ± | 25 ± | 18 | V (GUG) | +8 | 2^{nd} | |
| | | 37 | 5 | | | | | |
| 235 | −9.5 | 282 ± | 29 ± | 10 | L (CUG) | +5 | 4^{th} | |
| | | 13 | 5 | | | | | |
| 236 | −7.9 | 402 ± | 65 ± | 6.5 | L (UUG) | +3 | 3^{rd} | |
| | | 58 | 4 | | | | | |
| 238 | −8.4 | 367 ± | 86 ± | 4.4 | V (GUG) | +4 | 2^{nd} | |
| | | 41 | 5 | | | | | |
| 244 | −3.5 | 362 ± | 360 ± | 1 | M (AUG) | +4 | 1^{st} | |
| | | 54 | 5 | | | | | |
| 245 | −5.1 | 411 ± | 222 ± | 1.9 | M (AUG) | +7 | 1^{st} | |
| | | 48 | 11 | | | | | |
| 249 | −1.9 | 406 ± | 391 ± | 1.1 | M (AUG) | +7 | 1^{st} | |
| | | 50 | 18 | | | | | |

TABLE 2
RBS calculator predictions compared to observed measurements. Candidate
ribosome binding sequences (RBS), including their Shine Dalgarno (SD)
sequences, were predicted using the RBS calculator (19) that both
identifies and scores possible translation initiation sites, based on
the 30S binding model for de novo translation initiation. The de novo
initiation predictions showed no significant correlation with the ob-
served GFP levels(r² = 0.08), with the levels of expression observed
being generally more substantial than the predictions. This strengthens
the argument that the expression of the distal operon gene encoding
GFP to be mainly the result of re-initiation and not de-novo initiation.

| | | | Start | | | | |
|---|---|---|---|---|---|---|---|
| | | Best re- | codon | | | | RBS |
| | | initiation | position, | | | | binding |
| | | start codon | relative | | Observed | Predicted | energy |
| | | candidate | to stop | Codon | translation | translation | ΔGₜₒₜₐₗ |
| Clone | ΔG_{fold} | (s) | codon | rank | rate [AU] | rate [AU] | [kcal/mol] |
| 29 | −8.9 | I (AUU) | +8 | 6^{th} | 32 ± 4 | 0 | NA |
| 33 | −9.4 | V (GUG) | +8 | 2^{nd} | 48 ± 5 | 1.34 | 15.16 |
| 52 | −1.3 | M (AUG) | +5 | 1^{st} | 344 ± 12 | 90.75 | 5.80 |
| 56 | −8.5 | V (GUG) | +6 | 2^{nd} | 62 ± 4 | 6.31 | 11.72 |
| 57 | −6.5 | L (UUG) | +8 | 3^{rd} | 43 ± 7 | 0.49 | 17.40 |
| 62 | −6.7 | L (CUG) | +9 | 4^{th} | 39 ± 4 | 0 | NA |
| 71 | −4.9 | L (UUG) | +6 | 3^{rd} | 98 ± 4 | 13.79 | 9.98 |
| 91 | −2.5 | L (UUG) | +9 | 3^{rd} | 103 ± 4 | 0.96 | 15.91 |
| 96 | −2.1 | V (GUG) | +8 | 2^{nd} | 197 ± 7 | 5.76 | 11.92 |
| 101 | −6.0 | L (UUG) | +6 | 3^{rd} | 74 ± 24 | 35.90 | 7.86 |
| 110 | −8.2 | M (AUG) | +8 | 1^{st} | 57 ± 4 | 0.66 | 16.72 |
| 111 | −13.7 | I (AUU) | +5 | 6^{th} | 43 ± 3 | 0 | NA |
| 202 | −11.3 | V (GUG) | +4 | 2^{nd} | 38 ± 2 | 0.33 | 18.29 |
| 203 | −7.6 | L (UUG) | +7 | 3^{rd} | 33 ± 6 | 2.10 | 14.16 |
| 207 | −1.8 | M (AUG) | +3 | 1^{st} | 526 ± 53.5 | 16.52 | 9.58 |
| 208 | −1.9 | M (AUG) | +7 | 1^{st} | 140 ± 19 | 32.12 | 8.11 |
| 209 | −4.7 | M (AUG) | +7 | 1^{st} | 274 ± 8 | 479.88 | 2.10 |
| 212 | −5.9 | M (AUG) | +3 | 1^{st} | 137 ± 5 | 62.34 | 6.63 |
| 214 | −2.8 | M (AUG) | +3 | 1^{st} | 478 ± 17 | 11.74 | 10.34 |
| 216 | −0.8 | M (AUG) | +5 | 1^{st} | 193 ± 17 | 150.90 | 4.67 |
| 220 | −3.9 | M (AUG) | +6 | 1^{st} | 201 ± 16 | 1011.93 | 0.44 |
| 222 | −6.6 | M (AUG) | +3 | 1^{st} | 211 ± 13 | 1.78 | 14.53 |
| 225 | −7.5 | L (UUG) | +3 | 3^{rd} | 78 ± 4 | 1.61 | 14.76 |
| 226 | −9.2 | L (UUG) | +5 | 3^{rd} | 56 ± 6 | 0.70 | 16.59 |
| | | M (AUG) | +9 | 1^{st} | | 3.31 | 13.16 |
| 230 | −5.0 | M (AUG) | +8 | 1^{st} | 41 ± 6 | 3.72 | 12.90 |
| 232 | −7.6 | M (AUG) | +6 | 1^{st} | 36 ± 5 | 122.95 | 5.12 |
| 233 | −8.4 | V (GUG) | +8 | 2^{nd} | 25 ± 5 | 0.13 | 20.39 |
| 235 | −9.5 | L (CUG) | +5 | 4^{th} | 29 ± 5 | 0 | NA |
| 236 | −7.9 | L (UUG) | +3 | 3^{rd} | 65 ± 4 | 0.28 | 18.63 |
| 238 | −8.4 | V (GUG) | +4 | 2^{nd} | 86 ± 5 | 1.54 | 14.86 |
| 244 | −3.5 | M (AUG) | +4 | 1^{st} | 360 ± 5 | 272.86 | 3.35 |
| 245 | −5.1 | M (AUG) | +7 | 1^{st} | 222 ± 11 | 710.51 | 1.23 |
| 249 | −1.9 | M (AUG) | +7 | 1^{st} | 391 ± 18 | 98.94 | 5.61 |

### Example 2: The RTS is Conserved Across Bacterial Genomes

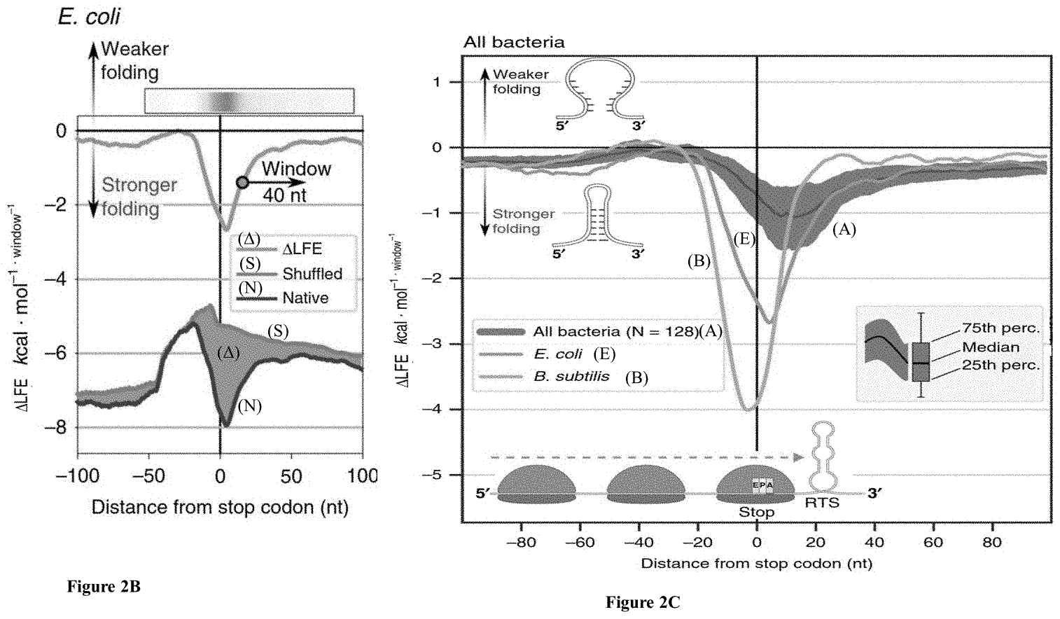

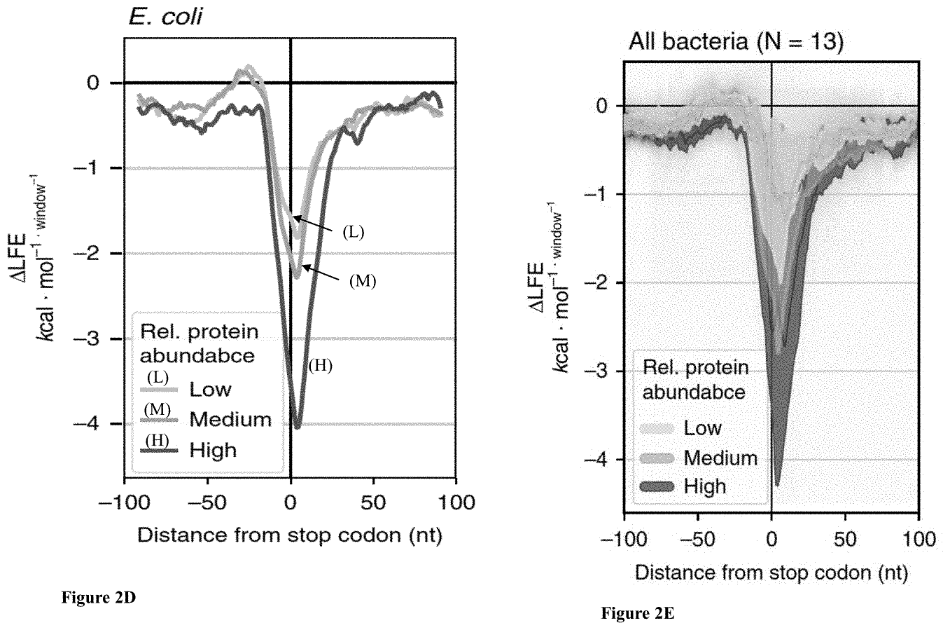

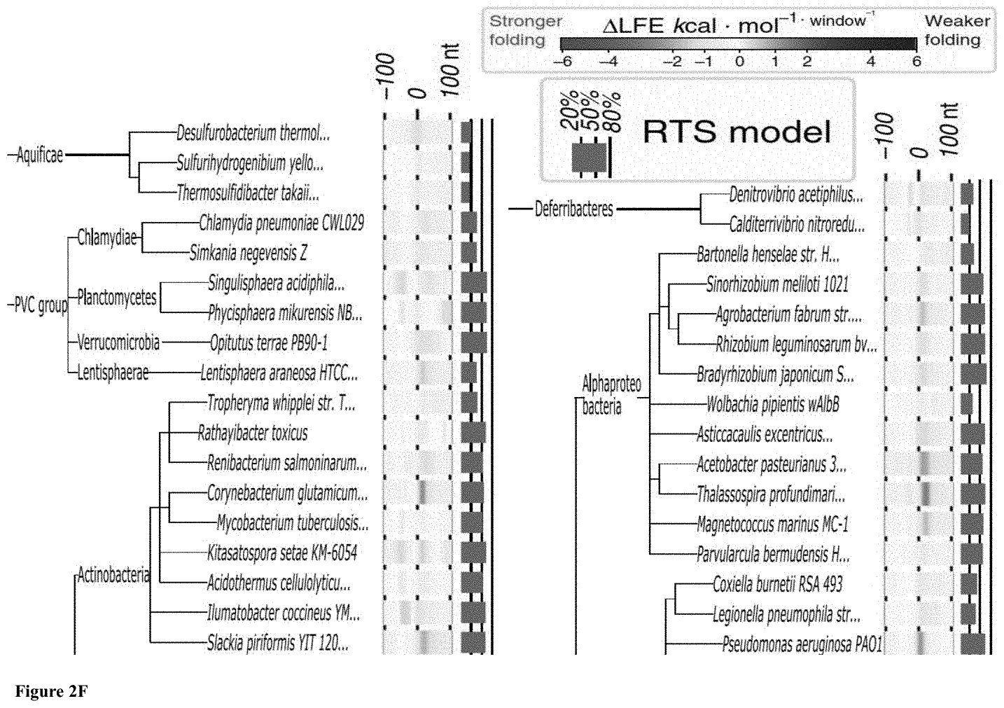

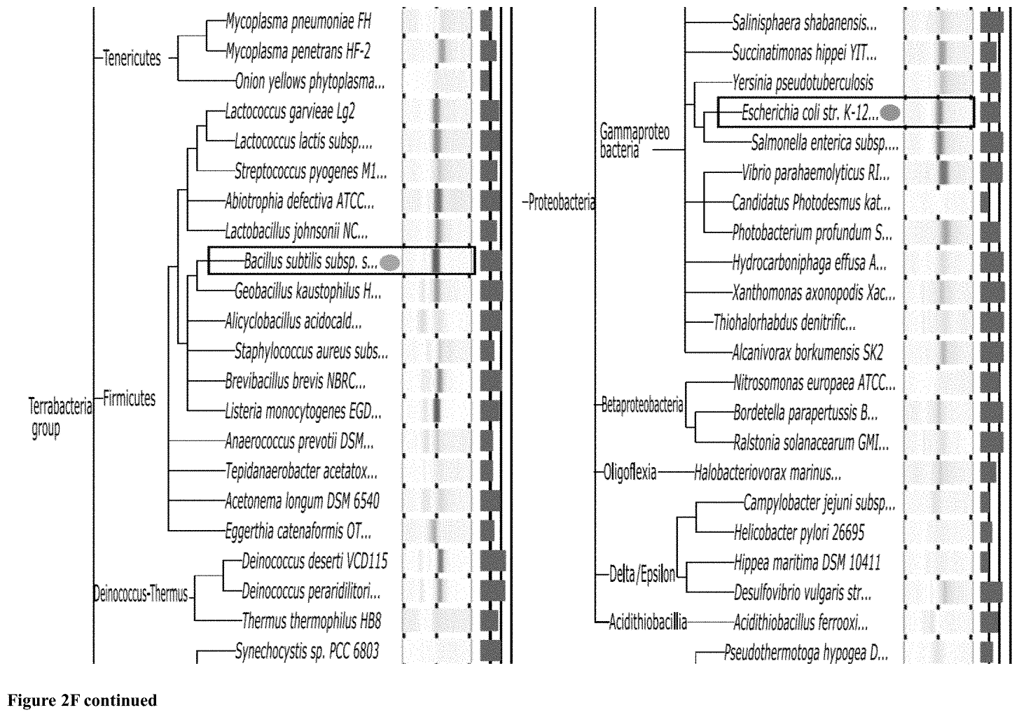

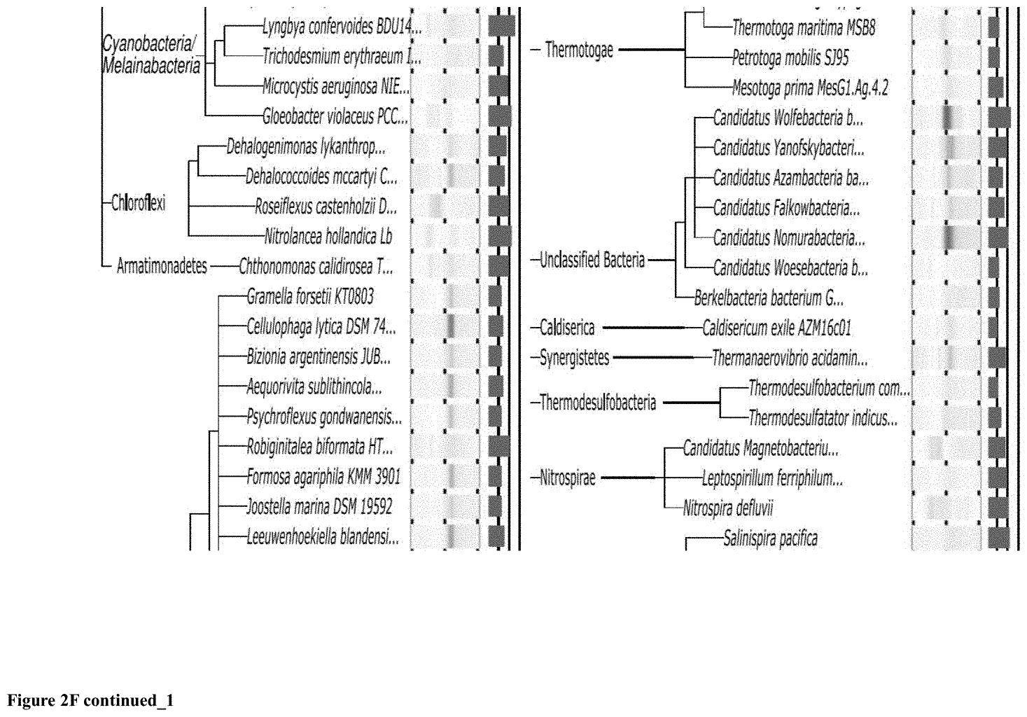

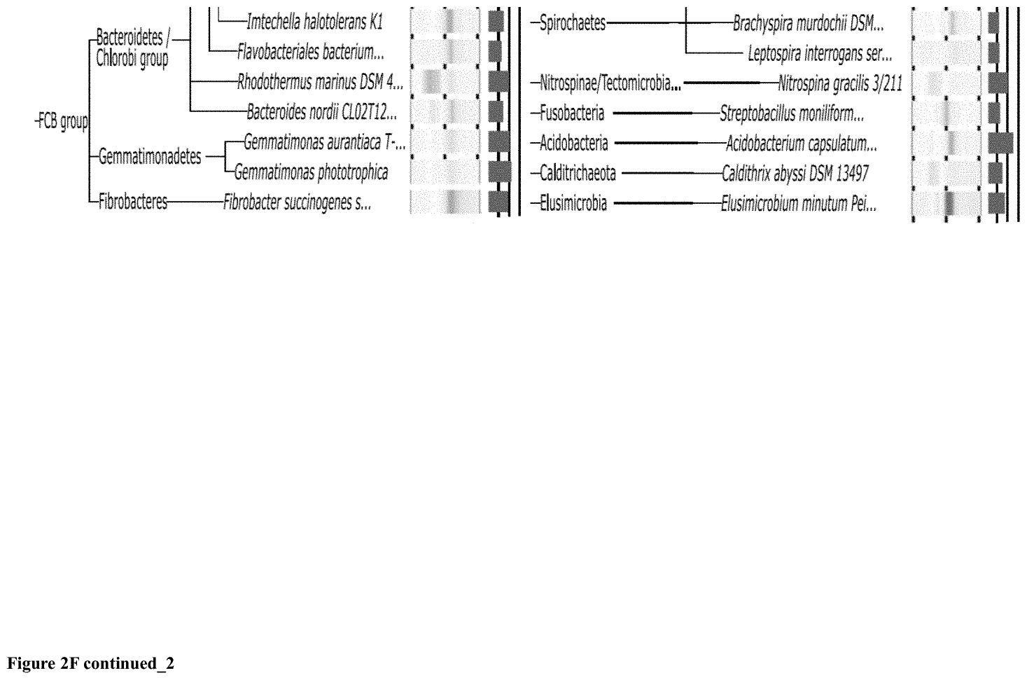

To assess the generality of the RTS, mRNA secondary structure stability (ΔG_{fold}) was calculated in a region spanning 100 nucleotides on either side of each of the ˜4,200 annotated E. coli stop codons using a 40 nucleotide-long sliding window, allowing for calculation of the mean ΔG_{fold} at each position in a genome-wide manner (FIG. 2A). Such analysis revealed an extreme drop in ΔG_{fold} (reflecting stronger mRNA folding), with a global minimum of −7.94 kcal mol⁻¹ window⁻¹ centered five nucleotides downstream of stop codons (FIG. 2B, blue line), corresponding to the expected position and magnitude and magnitude of an RTS. This demonstrates that RTS-like signals are apparent throughout the E. coli genome.

To confirm that the RTS is directly under selection and as a control for other mRNA-stability factors, the ΔG_{fold} value of each sequence (FIG. 2B, blue line) minus the ΔG_{fold} value of a shuffled version in which nucleotide and codon content but not their order are preserved, was calculated (FIG. 2B, green line). This was repeated for each position across all E. coli genes, providing an average selection landscape of mRNA structure (FIG. 2B, orange line). If only nucleotide or codon content were under selection, then the difference in local folding energy (ΔLFE) between the native and randomized sequences should equal zero. Hence, increased ΔLFE deviation in the negative direction indicates direct selection for enhanced secondary structure stability (and vice versa). The results reveal extreme selection for stable structure directly downstream of stop codons (FIG. 2B, orange line) (Wilcoxon test p-val<10⁻³⁰), irrespective of the stop codon used (FIG. 8A-D). The global minimum of ΔLFE (−2.67 kcal mol⁻¹ window⁻¹) represents strong selection for the RTS structure directly downstream of stop codons. The same signal was seen in an average of 128 other bacterial strains representing all phyla (FIG. 2C, blue line), including the evolutionary distant Gram-positive Bacillus subtilis (FIG. 2C, red line).

If RTS presence is indeed under selection, correlation to the level of gene expression would be expected, with genes encoding more abundant proteins being subjected to stronger selection pressure. To test this hypothesis, E. coli genes were grouped according to protein abundance, and the ΔLFE landscape of each was determined (FIG. 2D). Clear and significant correlation between protein abundance and ΔLFE was noted (Mann-Whitney test, p-value<10⁻³⁰), demonstrating the RTS to be an adaptive trait, controlling distal operon gene translation. This relation also holds true in B. subtilis and all 11 other bacteria for which data is available (FIG. 2E).

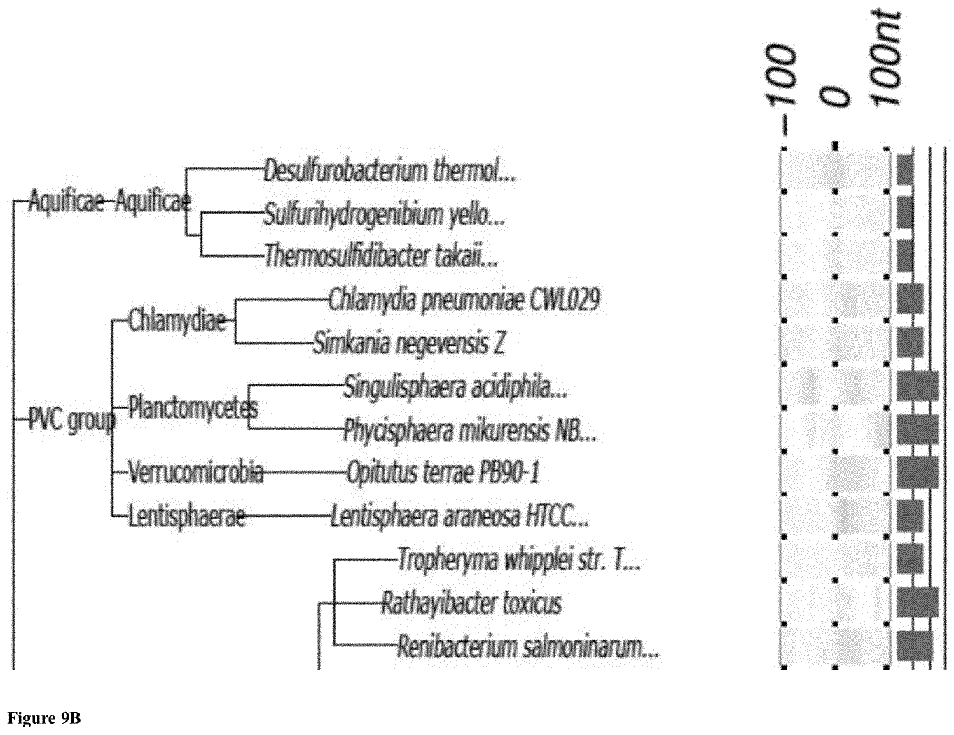

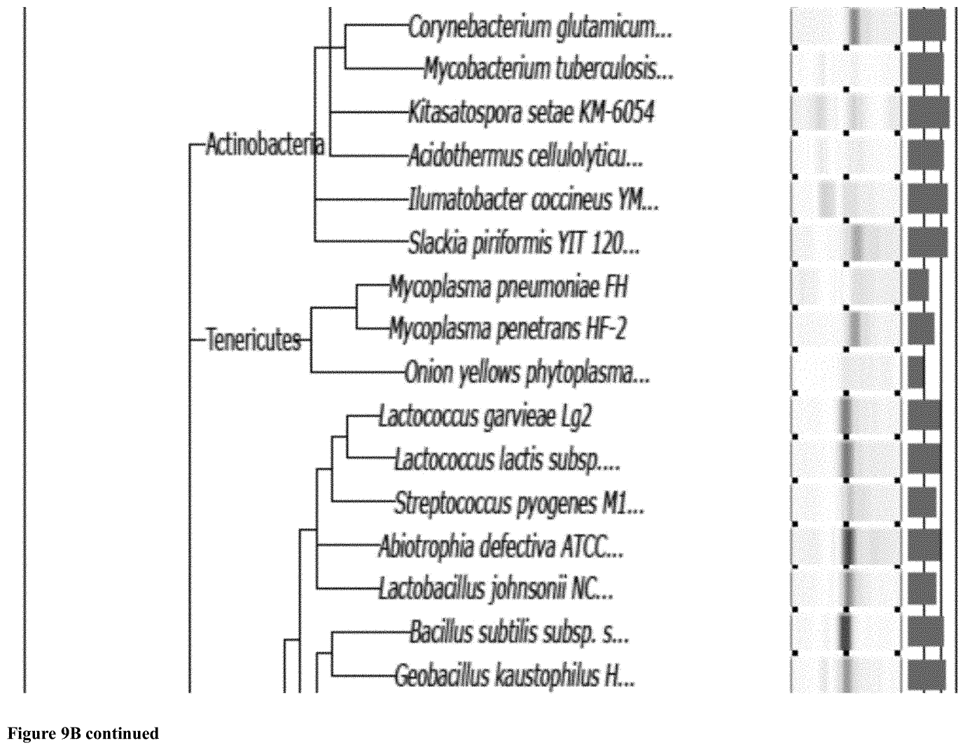

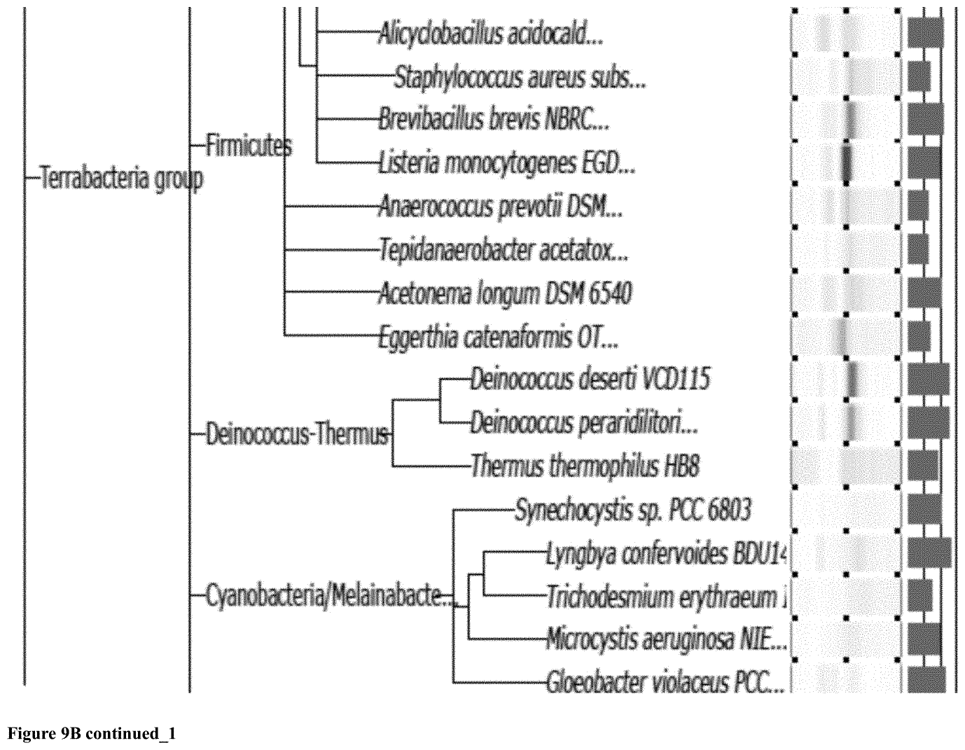

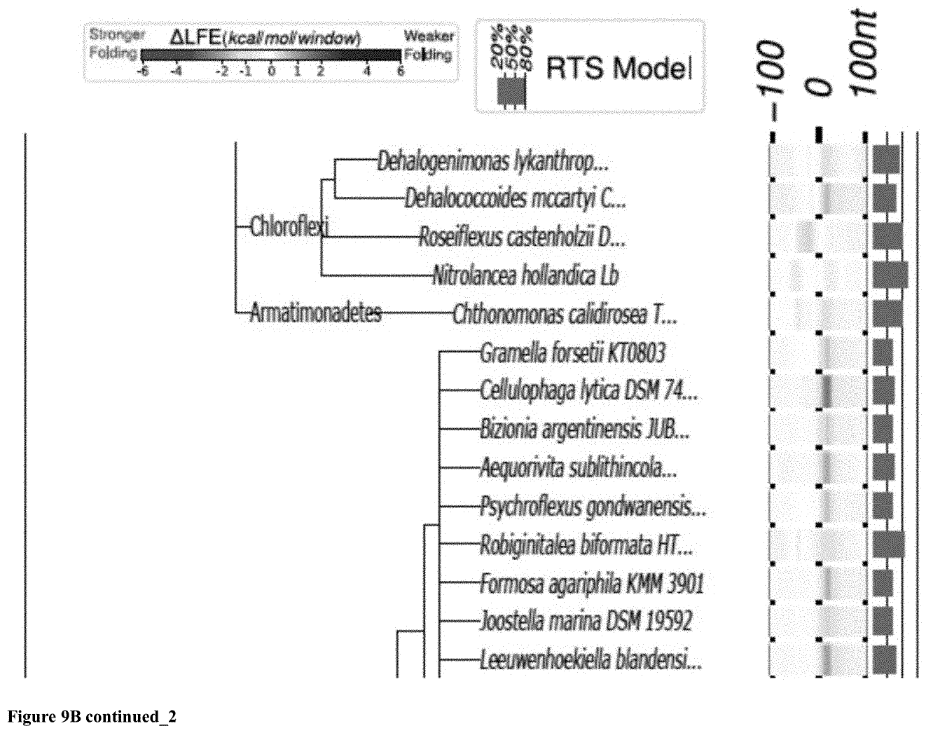

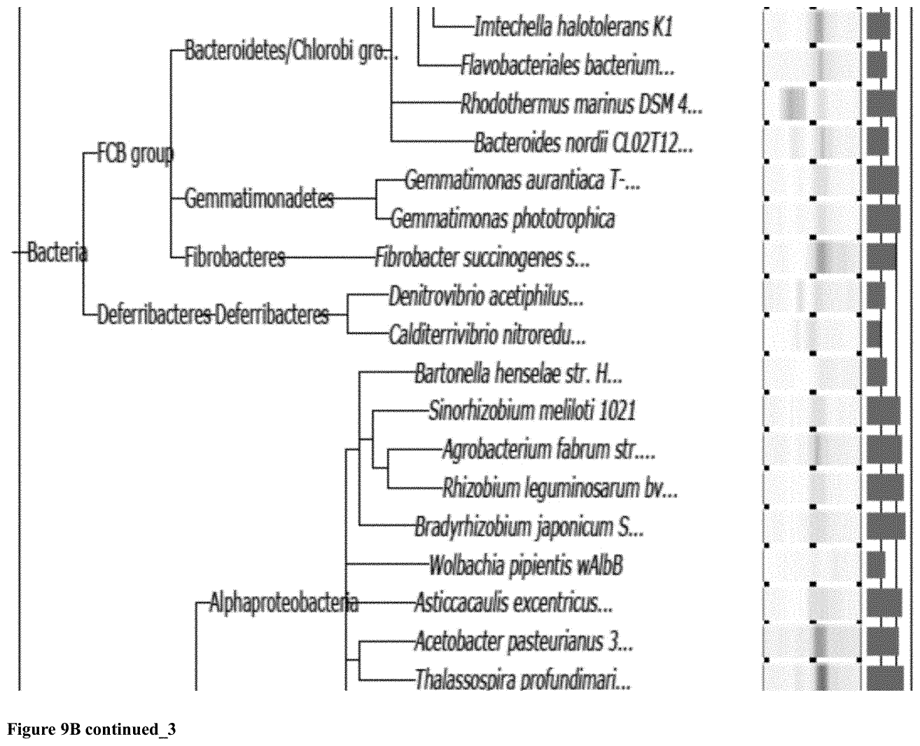

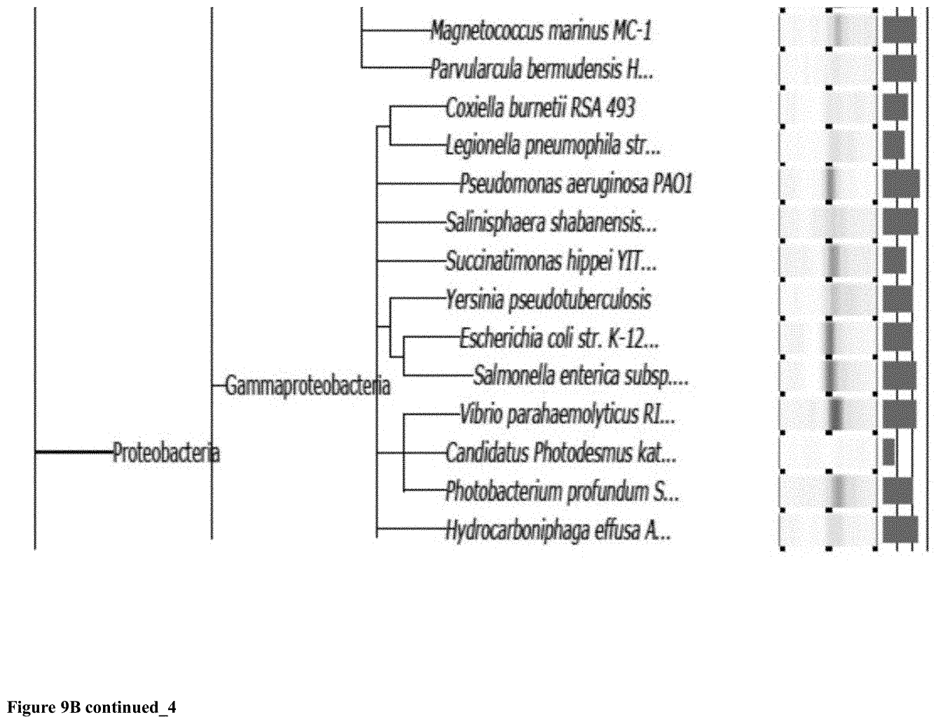

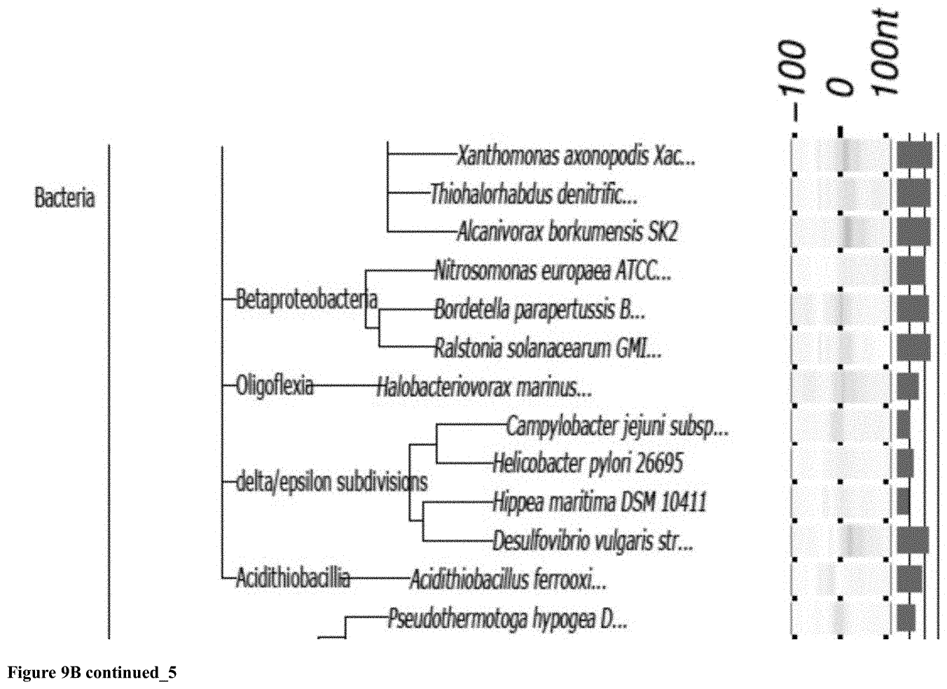

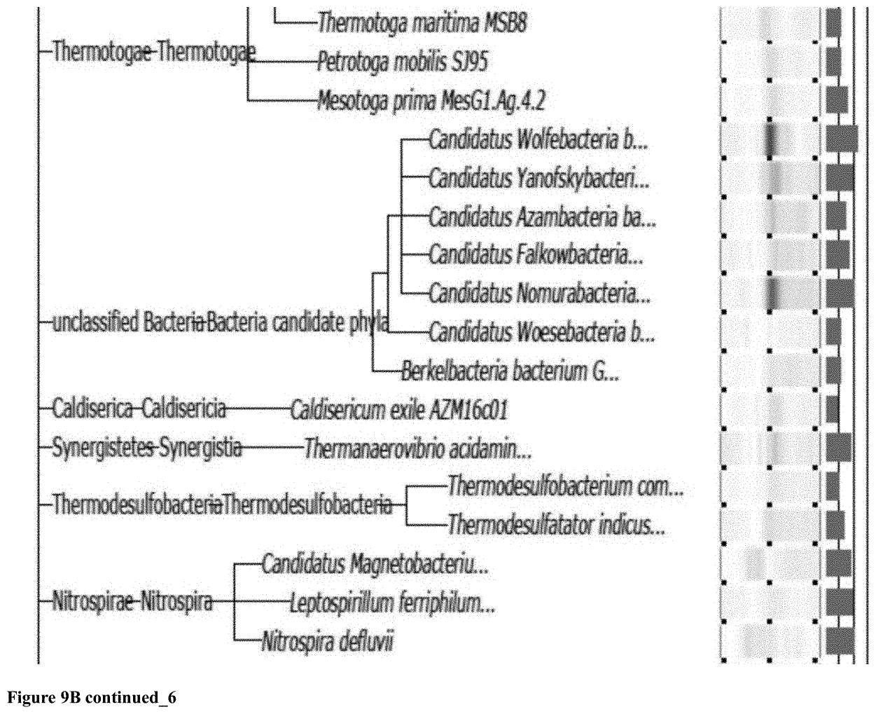

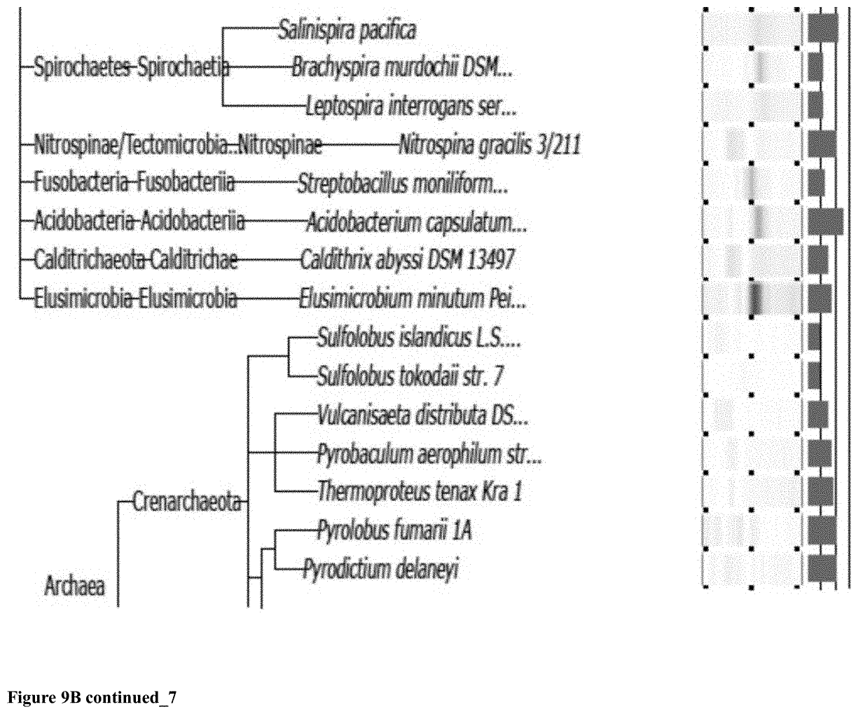

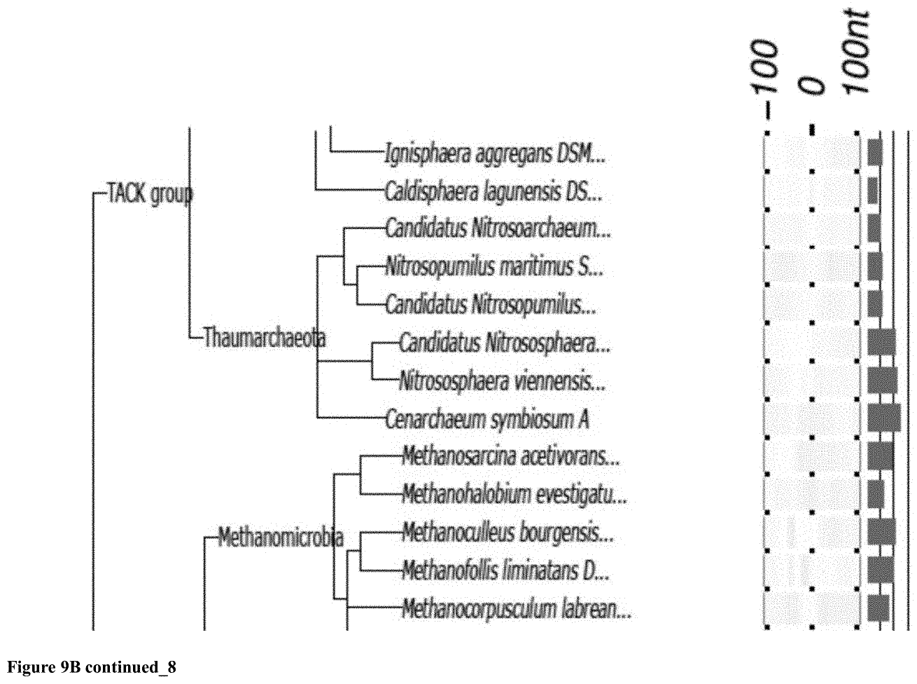

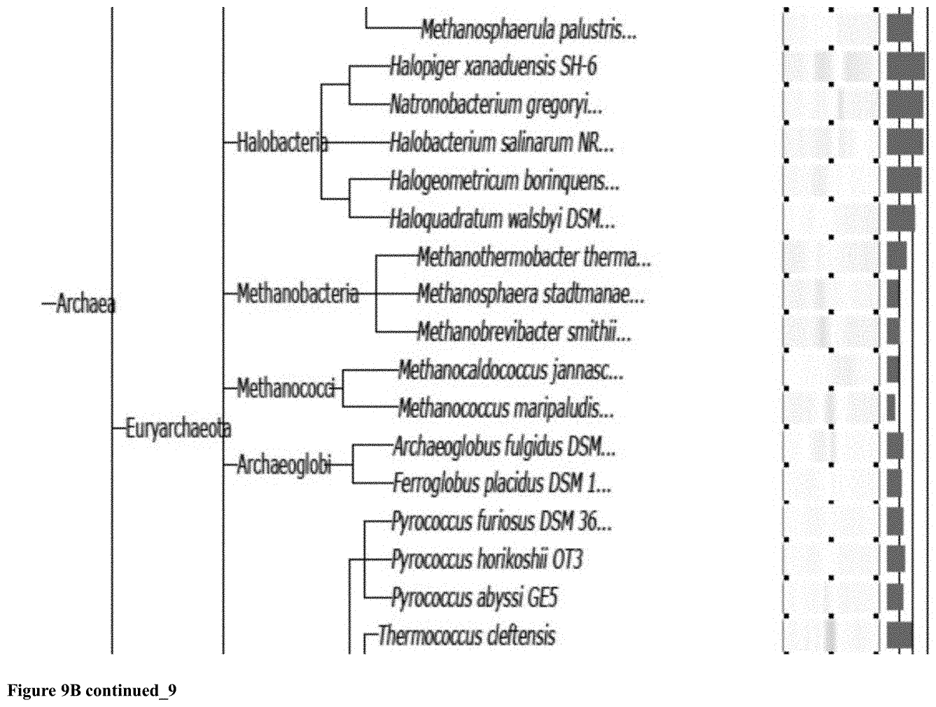

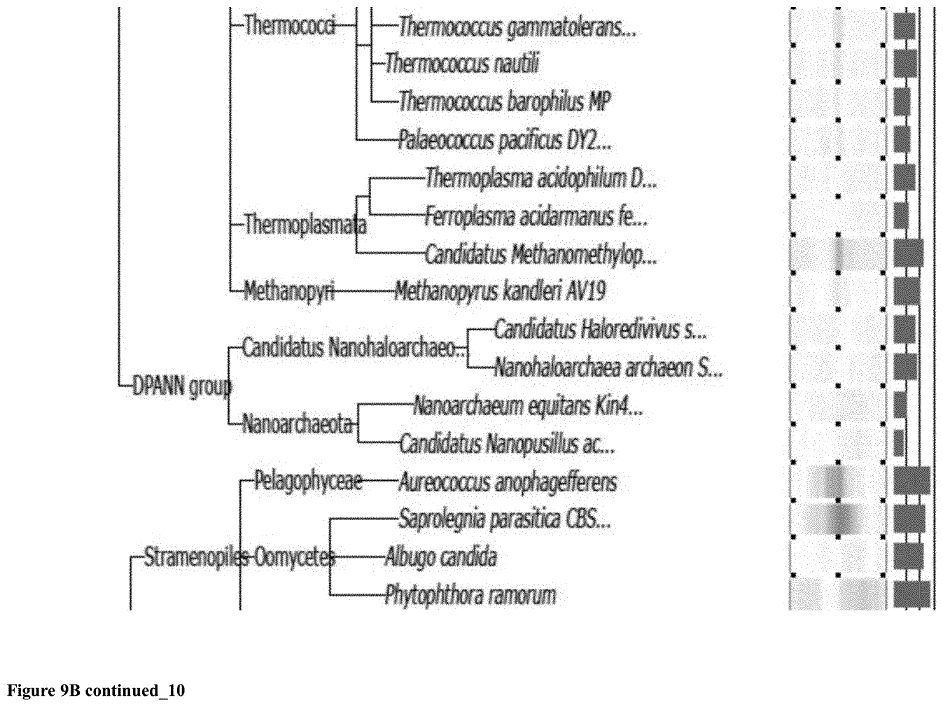

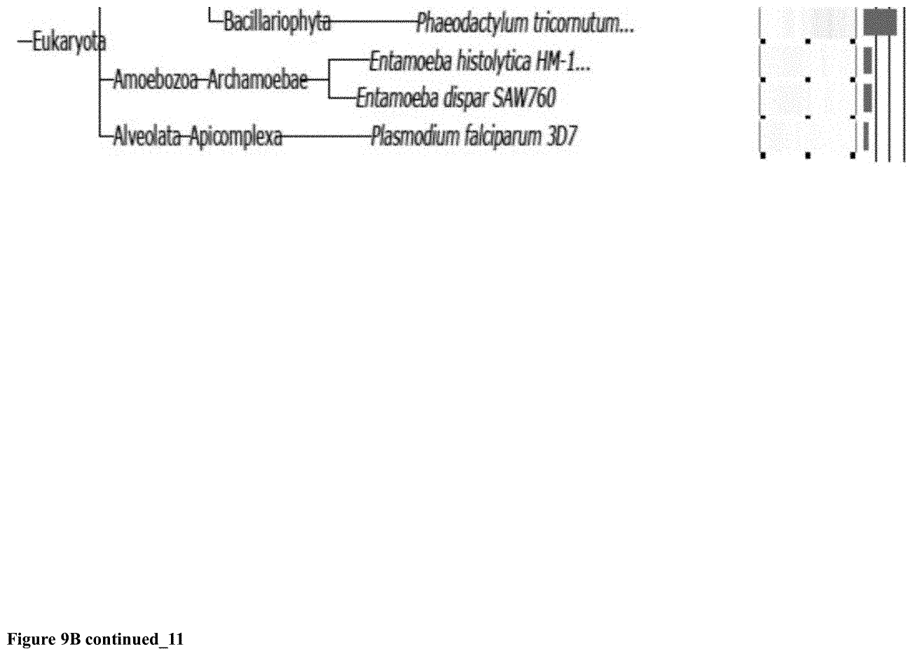

Lastly, RTS presence was quantified genome-wide across bacteria. This revealed that an RTS signal, defined by an mRNA structure (ΔG_{fold}≤−6 kcal mol⁻¹ window⁻¹) directly downstream of the stop codon that is significantly more stable than the surrounding sequences (see Materials and Methods), is present in 18%-66% of all genes, depending on the species (FIGS. 2F, and 9A-B). Genome-wide variability between species reflects a combination of selection for structural stability and the fraction of genes that are followed by an RTS.

### Example 3: Translation Re-Initiation is Controlled by RTS

The precise role of the RTS was considered by examining variability in ΔLFE, distinguishing between genes followed by an RTS or not. Such analysis showed the standard deviation of ΔLFE to spike in the vicinity at the stop codon (FIG. 3A), yielding a bi-modal pattern of gene distribution only around the stop codon (FIG. 3B). The parameter best defining the two groups of gene distribution is the inter-cistronic distance separating neighboring genes (FIG. 3B, inset). E. coli gene pairs separated by shorter distances (<25 nucleotides, n=1,537) were significantly depleted of RTSs (mean ΔLFE=+0.4 kcal/mol⁻¹, Wilcoxon test, p-value=5×10⁻¹⁹); for further-separated neighboring genes (≥25 nucleotides, N=2,581), RTSs were significantly enriched (mean ΔLFE=−4.0 kcal/mol⁻¹, Wilcoxon test, p-value<10⁻³⁰).

When the ΔLFE landscape around the stop codon between gene pairs in each group was charted (FIG. 3C), RTS depletion was noted when the intergenic distance is short, or when the two consecutive cistrons overlap. Conversely, when the intergenic distance exceeds 25 nucleotides, an RTS is present (Mann-Whitney, p-value<10⁻³⁰). This trend is conserved in 128 bacterial species analyzed (FIG. 3D). Considering that ˜25 nucleotides is the intergenic distance below which translation re-initiation is considered to be advantageous over de novo initiation, and the above-identified correlation between RTS presence and expression of the distal operonic GFP gene (FIG. 1), the RTS can be linked-to translation re-initiation. It is thus apparent that RTS enrichment in the 25 nucleotides group and depletion from the <25 nucleotides group reflects how RTS presence serves to inhibit translation re-initiation when it is not advantageous, while its absence enables this event.

Translation of the distal partner of any operon-based gene pair can be realized by de novo initiation, translation re-initiation, or stop codon read-through. Thus, discounting a link between the RTS and de novo initiation or stop codon read-through would further support a role for the RTS in translation re-initiation. Accordingly, experiments involving the synthetic operon described above (FIG. 1A) were performed, given how expression of the distal GFP gene could result from any of the above-mentioned processes.

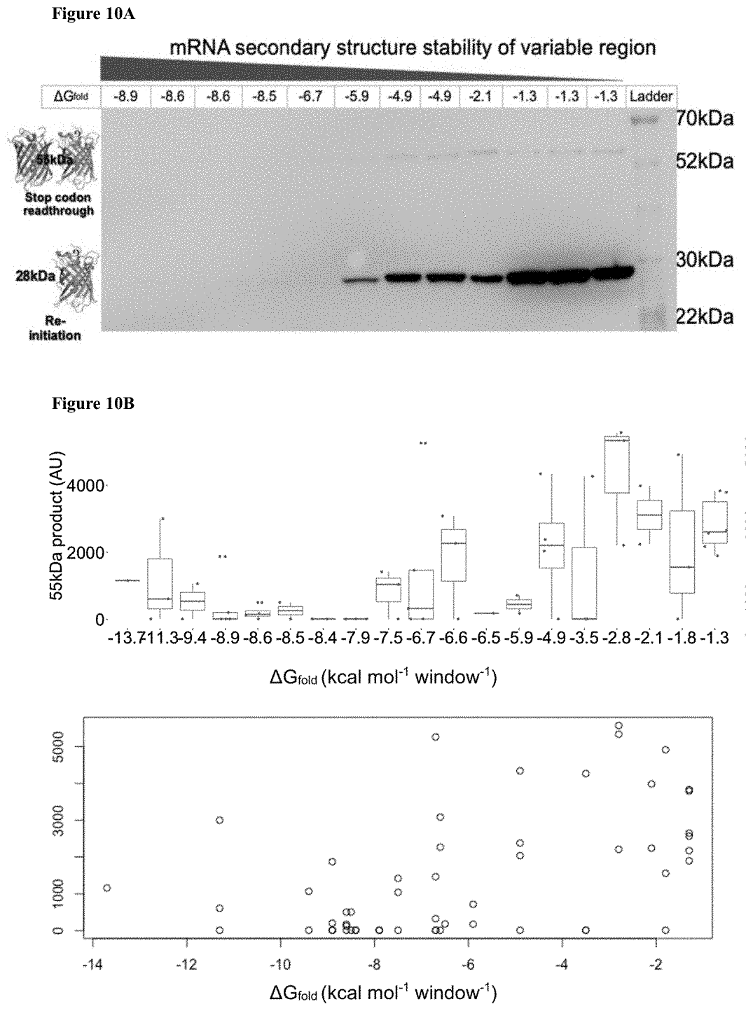

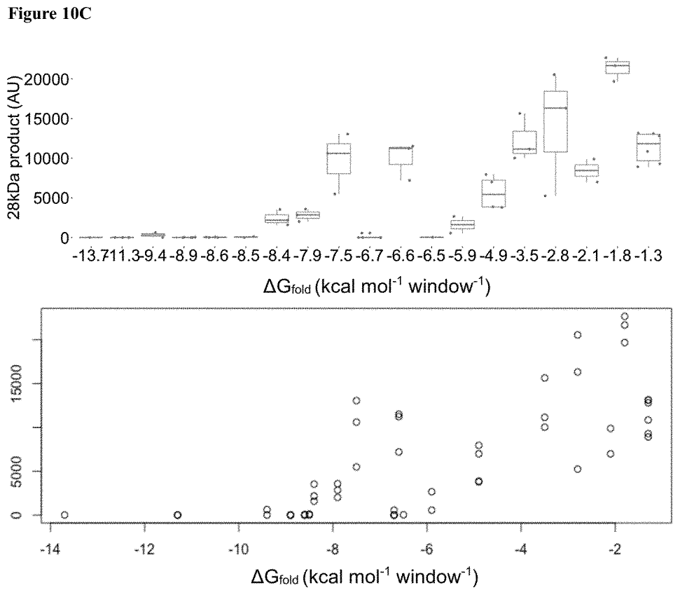

The link between the RTS and stop codon read-through was tested by Western blot analysis of a subgroup of clones described above (FIG. 1F) expressing RFP-GFP operon, normalized by OD₆₀₀, using antibodies against the GFP C-terminal poly-histidine tag. The 55 kDa RFP-GFP product resulting from stop codon read-through was barely detectable, compared to the 28 kDa GFP product resulting from de novo initiation or re-initiation (FIG. 3E). The intensities of these SDS-PAGE protein bands obtained from these clones, as well as those from other randomly selected clones, were quantified by densitometry. This confirmed that correlation between the level of the 28 kDa product and ΔG_{fold} was maintained (Spearman correlation ρ=0. 80, n=58, S=6,479 p-value<10⁻¹³) (FIG. 10A-C). Lastly, exact product masses were verified by mass spectrometry to reveal the initiation codon and its location (FIG. 3F, FIG. 10A-C, Table 1). These findings thus discount linkage between RTS presence and stop codon read-through.

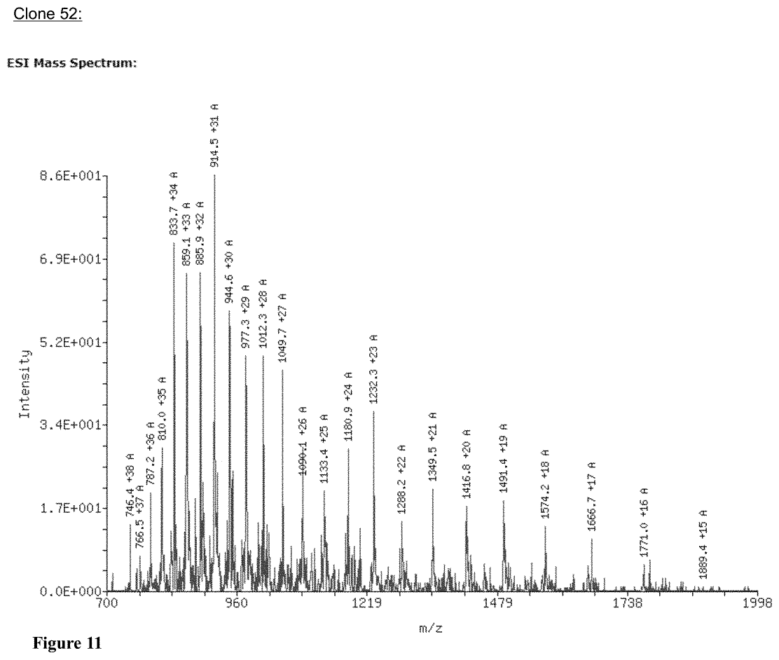

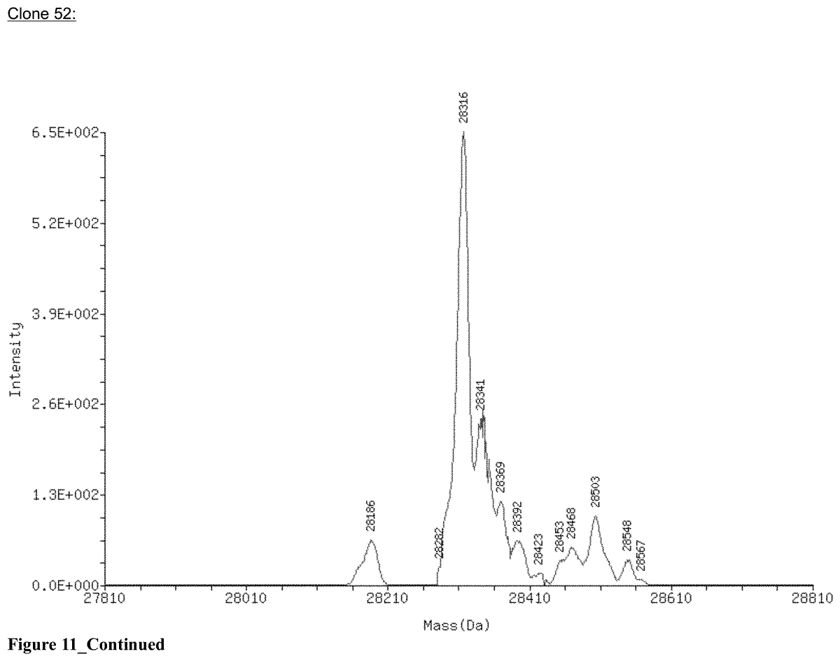

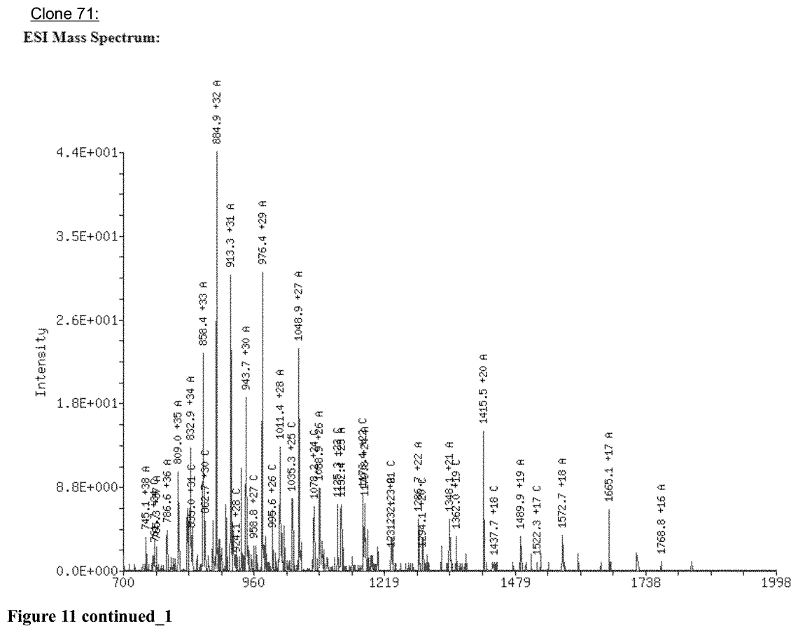

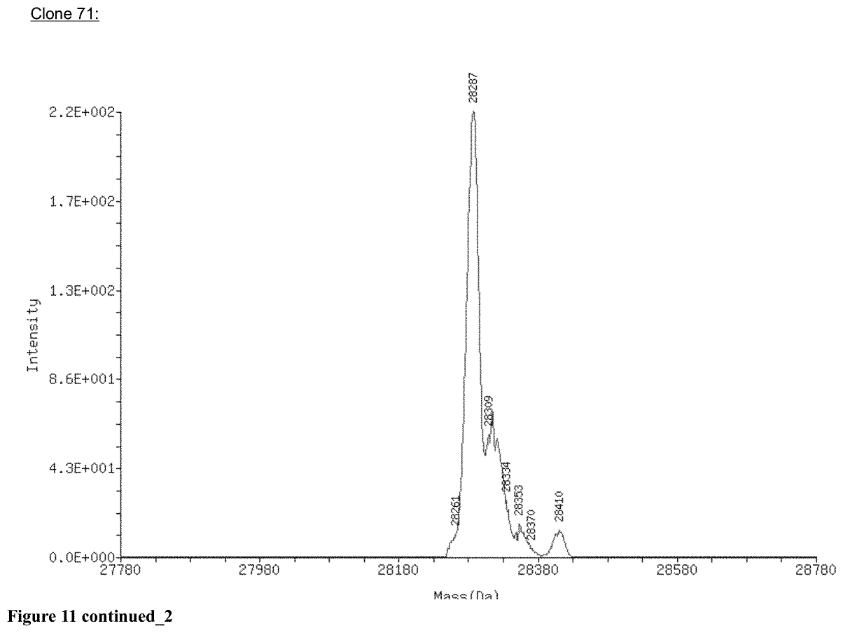

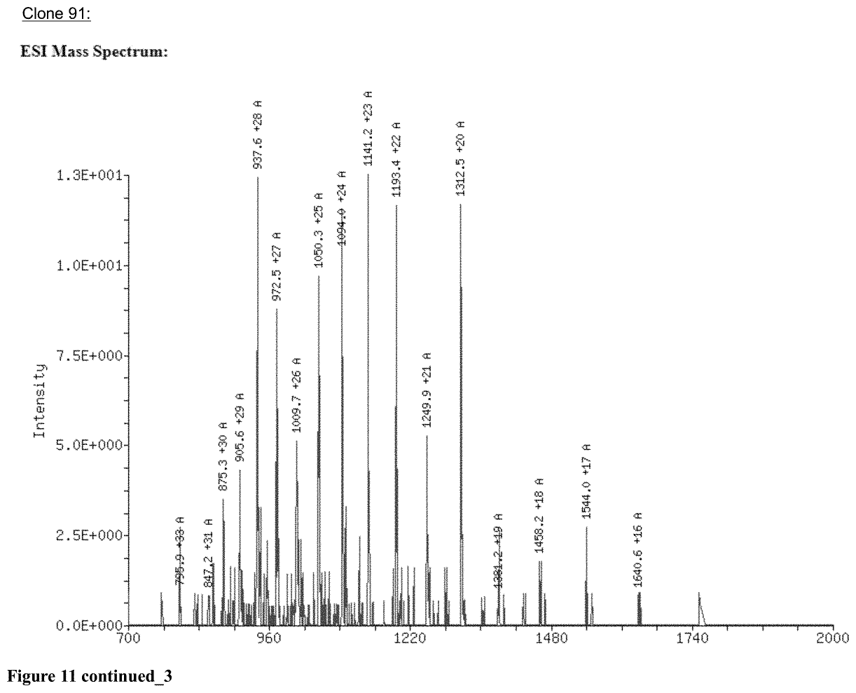

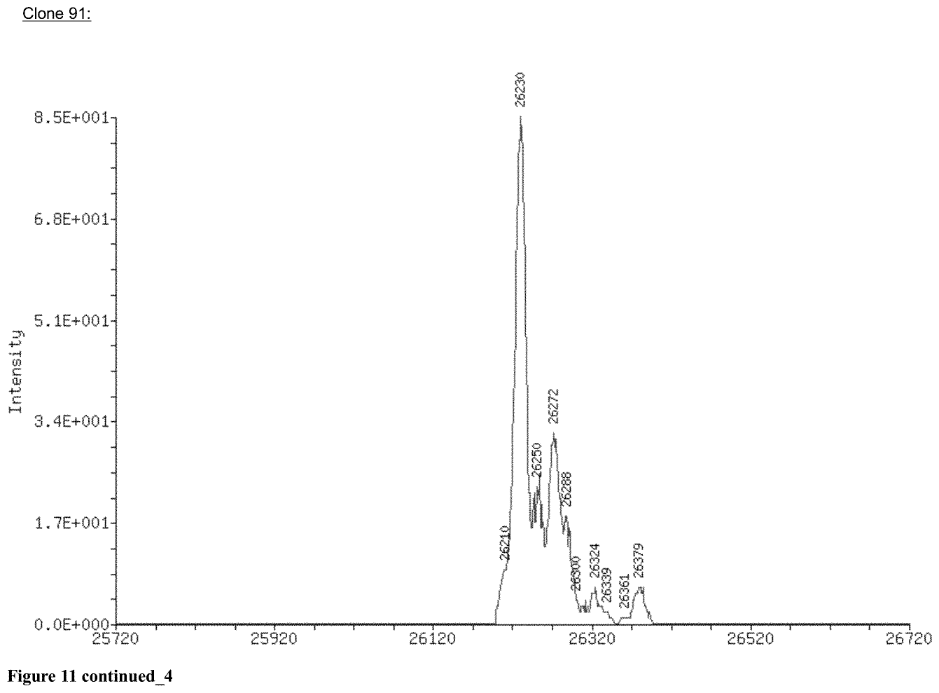

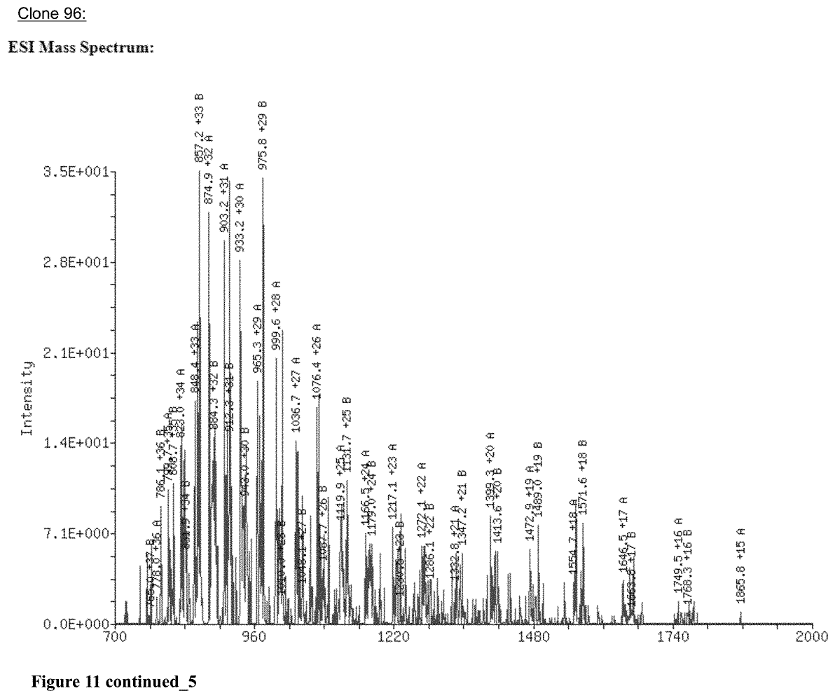

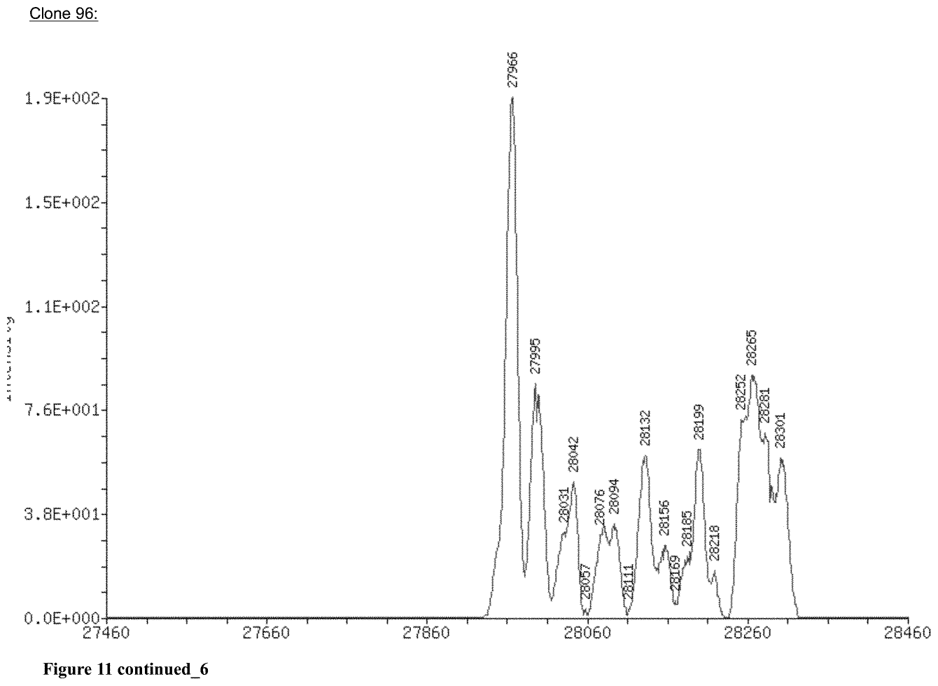

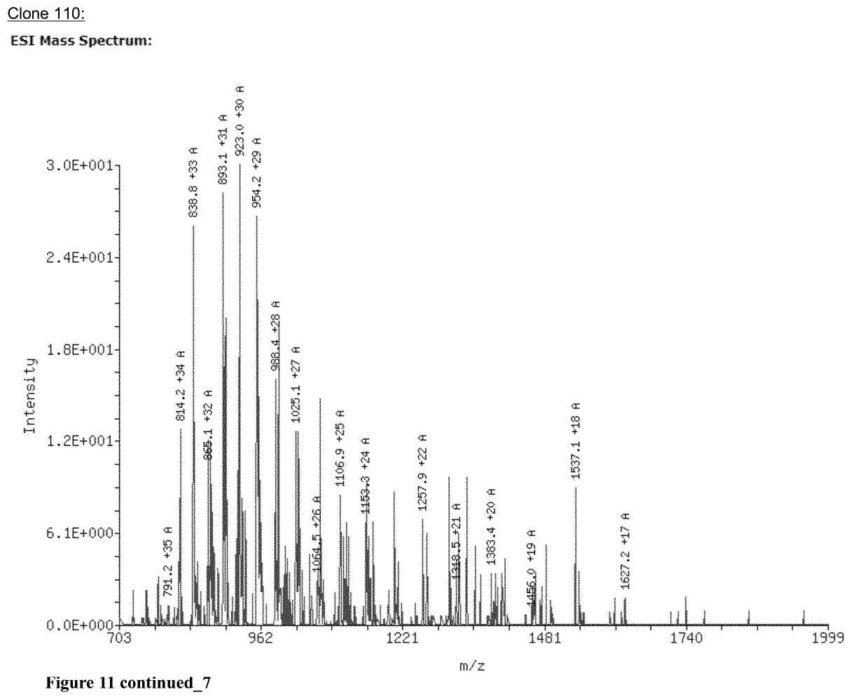

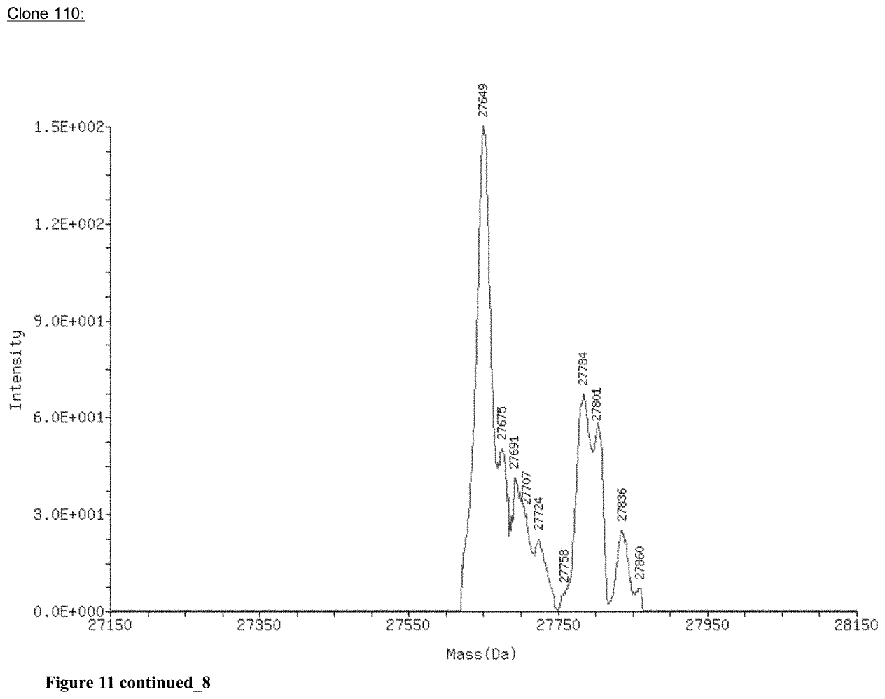

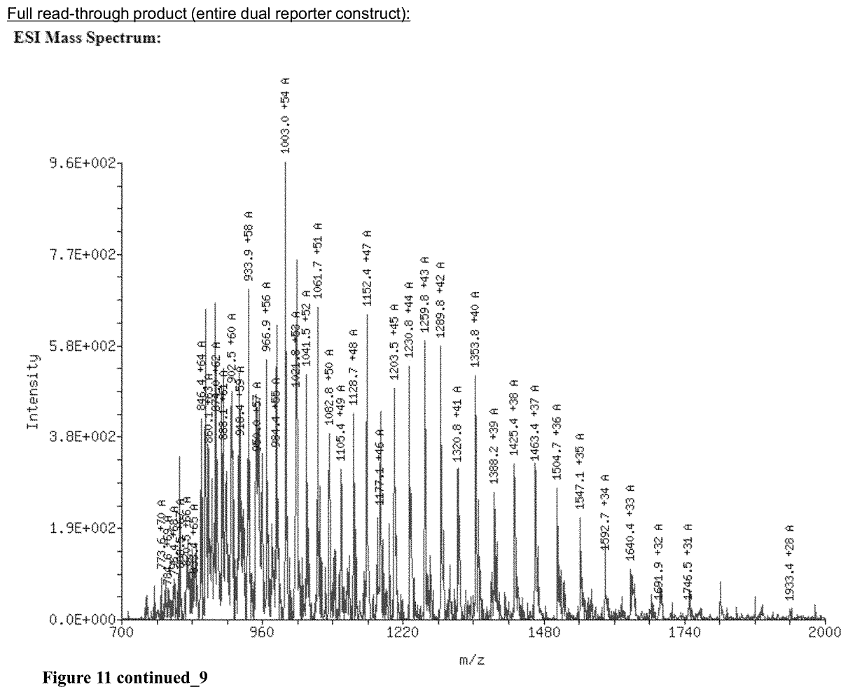

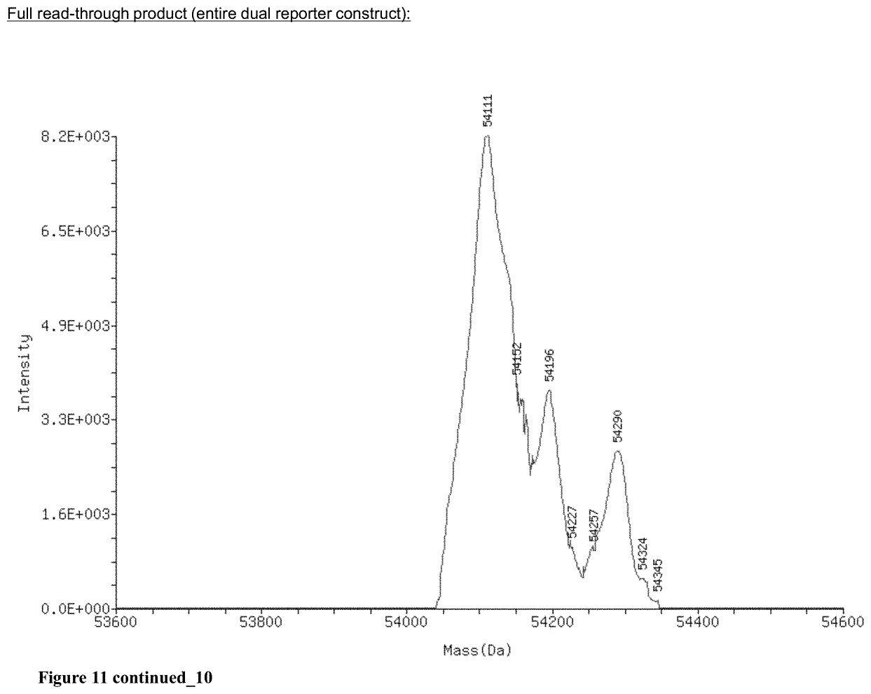

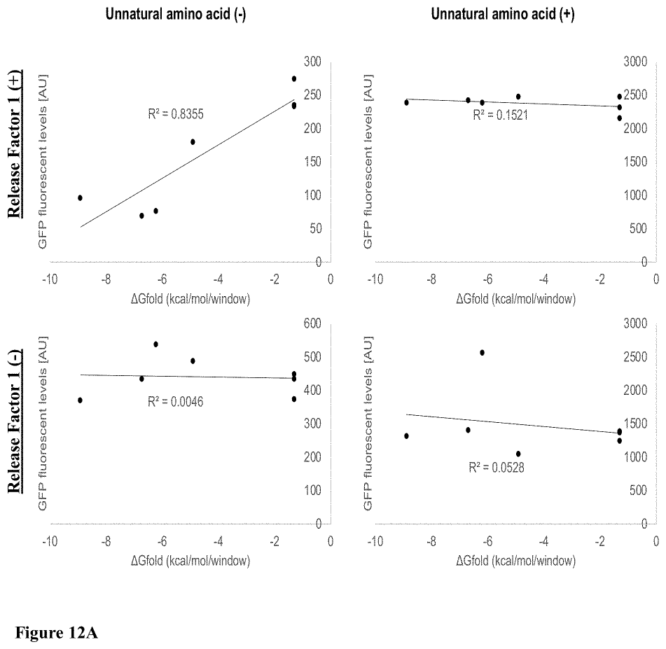

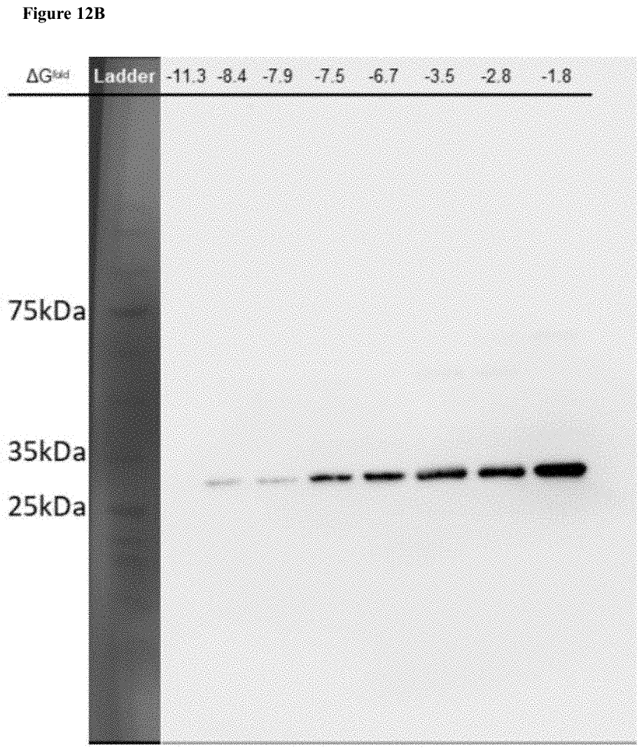

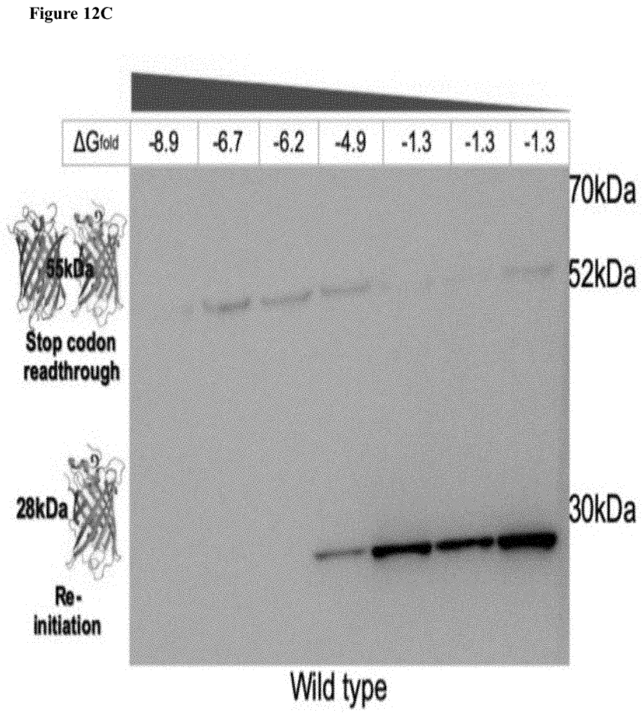

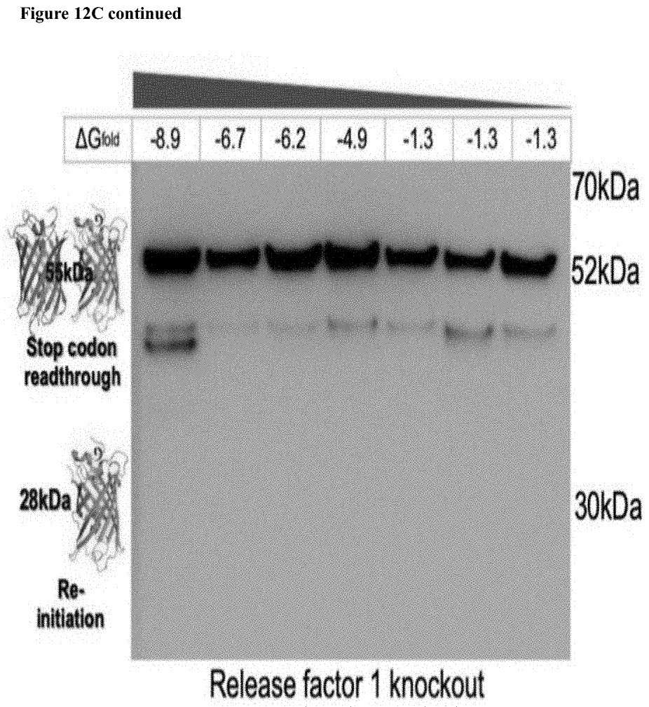

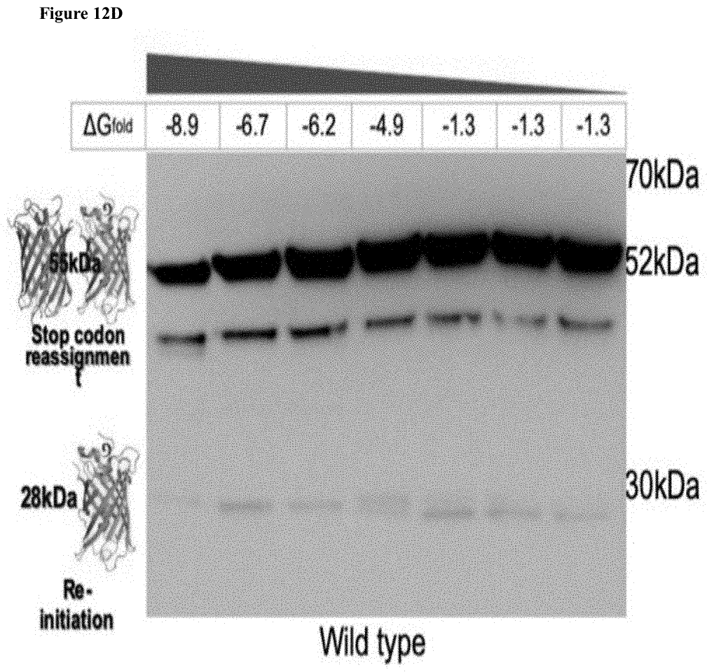

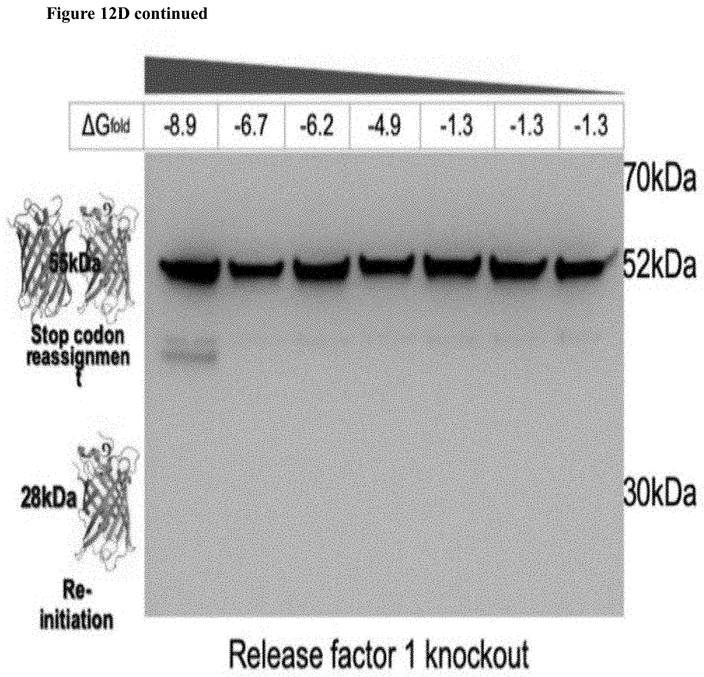

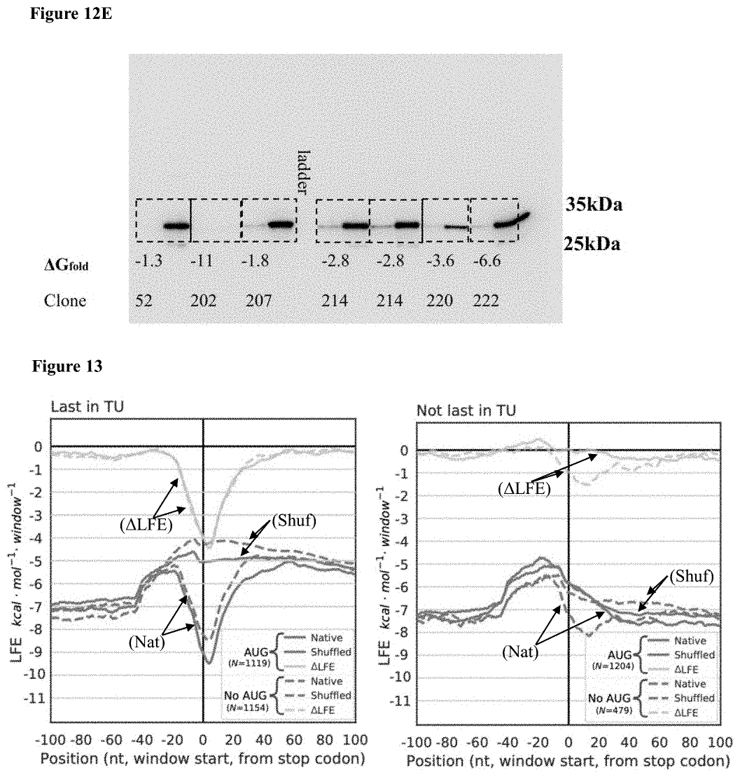

To determine whether the RTS is linked to de novo initiation or translation re-initiation, the manner of GFP translation initiation was assessed using the release factor 1 (RF1)-deficient E. coli C321.ΔprfA EXP strain and Western blot analysis of random clones, as above. In the absence of RF1, the ribosome cannot efficiently terminate translation at the RFP UAG stop codon, thereby precluding translation re-initiation, which depends on such termination. Instead, GFP expression can only be driven by read-through or de novo initiation in the mutant strain. Western blot analysis detected only the read-through RFP-GFP product (FIG. 3G, FIG. 11). This serves as evidence that de novo initiation does not drive GFP translation. Still, the apparent lack of de novo GFP translation initiation in the deletion strain could result from physical interference of the initiation site by RFP-translating ribosomes and increased read-through. To discount this possibility, the RFP UAG stop codon in E. coli MG1655 was suppressed (see Materials and Methods) so as to mimic conditions of ribosomal occupancy that may occur in RF1-deficient cells. Under these conditions, isolated GFP was produced only in the E. coli MG1655 strain but not in RF1-depleted cells (FIG. 3H).

Next, to directly test the ability of the intergenic region to guide de novo initiation of translation, the RFP gene and its ribosome-binding site were deleted from the operons in six selected clones. In the resulting monocistronic GFP construct, only the 18 terminal nucleobases of the RFP gene, the fixed and variable intergenic regions, and the GFP gene that directly follows the lac operator remain (FIG. 3I). The 18 terminal nucleobases of the RFP gene were left to mimic the exact mRNA sequence-context encountered by initiating ribosomes in all clones. GFP levels were then compared between the monocistronic and operonic constructs of each clone, using both Western blot analysis (FIG. 3I) and fluorescence measurements (FIG. 3J).

The results revealed that when strong RTSs are present, both constructs exhibit similarly low levels of GFP expression, with the ratio of expression by the two being close to one. Conversely, in clones with weak RTSs, the operonic constructs showed significantly higher levels of GFP expression, reaching levels over five-fold higher than that of the monocistronic constructs. This observation correlates well with the ΔG_{fold} of each pair of clones (FIG. 3K) (Spearman correlation ρ=0.94, S=2, n=6, P=0.017). Such correlation indicates that when the RTS is less stable, the difference in GFP expression between monocistronic and operonic constructs increases, as expected according to the hypothesis that a weak RTS allows for increased translation re-initiation. These results thus demonstrate how de novo initiation is not affected by the RTS in the same manner as is translation re-initiation. Moreover, they show that the monocistronic clones recruited new ribosomes for translation initiation with very low efficiency. This low efficiency confirms that a significant part of the observed GFP expression phenotype is dependent on the presence of the upstream RFP gene and, as such, is not likely a result of de novo initiation.

The fact that de novo initiation does not correlate with RTS strength, does not result in efficient expression in the monocistronic clones tested, and could not be detected when RF1 was knocked out, argue against de novo initiation as a viable mechanism to explain the dependence of operonic distal GFP expression on the RTS. As such, it was concluded that translation re-initiation is the process by which the RTS controls expression of the operonic distal GFP gene.

### Example 4: RTS is Dependent on the Operonic Position of a Gene

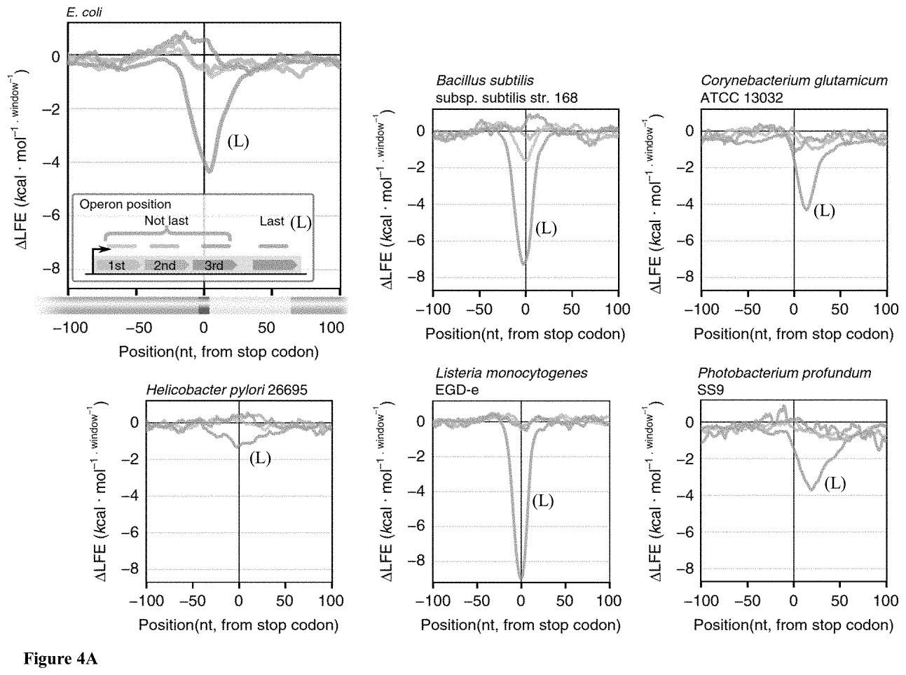

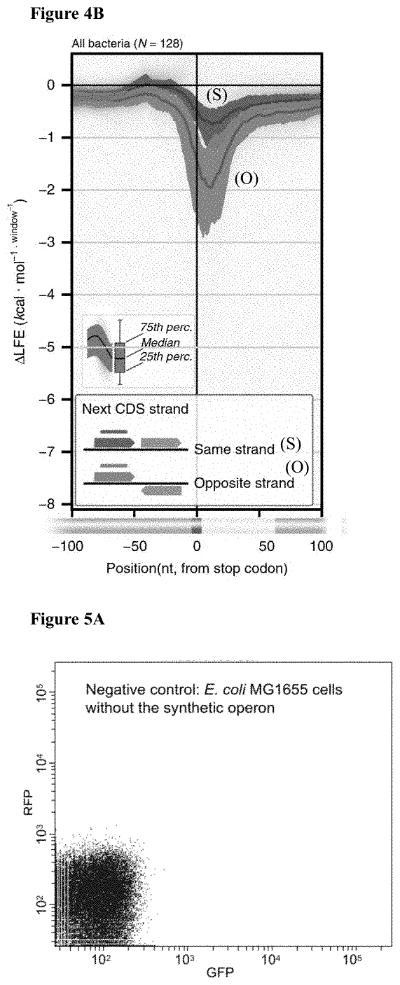

Finally, to determine whether the translation re-initiation-controlling role assigned to the RTS can be generalized, “transcriptional unit” data cataloging the arrangement of E. coli genes into operons was assessed (FIG. 4A).

Such analysis revealed that downstream of all operon terminal genes, where re-initiation is deleterious, the presence of an RTS after the stop codon, possibly insulating against re-initiation, is favored. In contrast, RTSs are depleted after the stop codon of all other operonic genes, thus encouraging re-initiation (Mann-Whitney, p-value<10⁻³⁰). These results were strengthened by observing that RTS presence after terminal operonic genes is independent of the presence or absence of start codons in the 50 nucleotide-long stretch downstream of the stop codon, while significant, such dependence was seen for other operon genes (FIG. 13). The same held true in B. subtilis and four other bacterial species for which experimental operon arrangement data exists (FIG. 4A).

Gene annotations in 128 bacterial species were analyzed for RTS presence as a function of neighboring gene strand directionality. Such analysis allowed for assessing operons in genomes where no operons are annotated, based on the assumption that neighboring genes on opposite DNA strands are less likely to be on the same operon than are gene pairs on the same strand. Accordingly, pairs of neighboring genes on the same strand, where re-initiation on the mRNA is possible, were compared to pairs on opposite strands, where such re-initiation would be useless as the two genes cannot be translated on the same mRNA (FIG. 4B). As expected, RTS presence was significantly higher within gene pairs found on opposite strands, where insulation against re-initiation could help avoid translation of the 3′ UTR in the downstream partner.

With this understanding, the source of variability between species in terms of the strength of selection for the RTS (i.e., ΔLFE values) was explored. This was performed for each of the 128 bacterial species considered, by distinguishing between gene pairs presenting intergenic distances of less than 25 nucleotides or which are on the same strand (i.e., where an RTS is less likely), and gene pairs separated by larger intergenic distances or found on opposite strands (i.e., where an RTS is more likely).

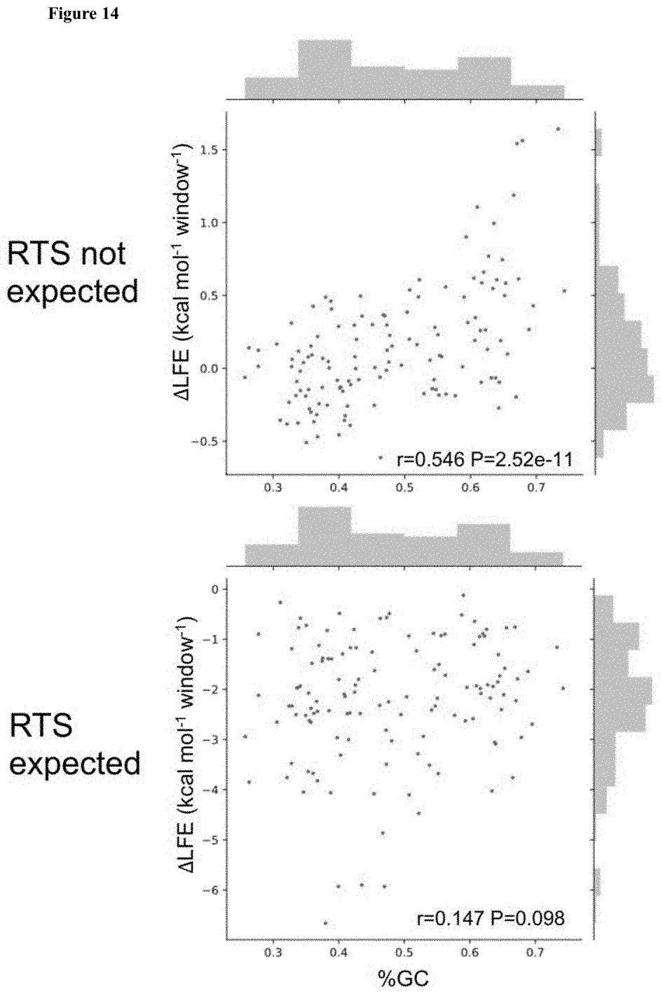

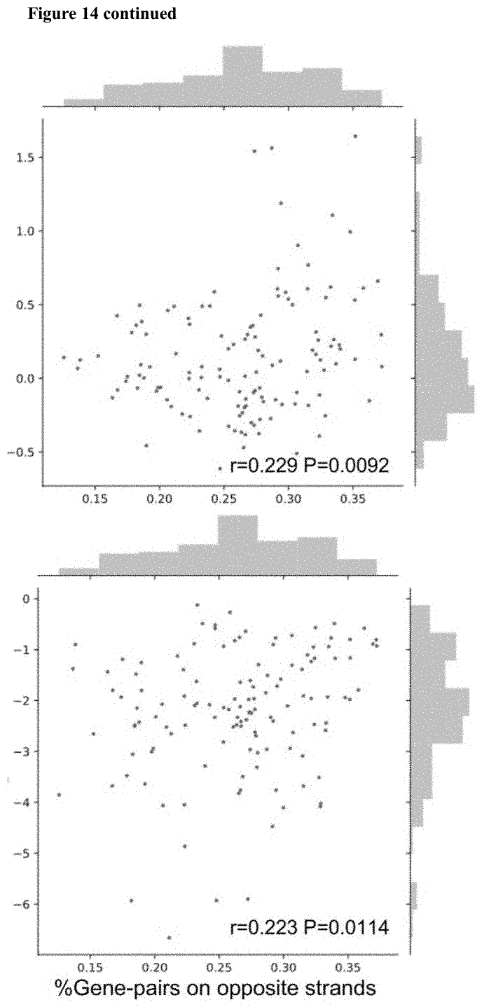

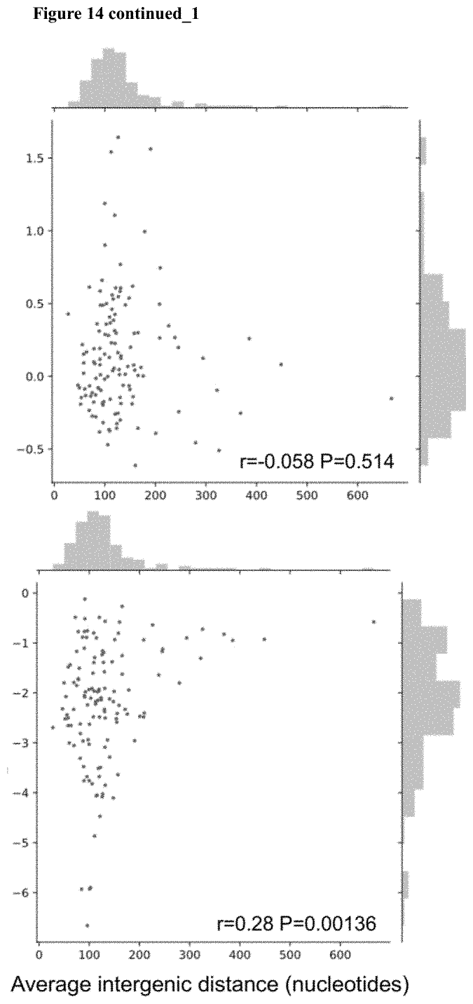

Three genome-specific parameters were examined, namely, % GC content, the number of gene pairs on opposing strands, and the average intergenic length (FIG. 14). Although inter-species variance in RTS selection was found to be correlated to all three parameters, it is of note that the high positive correlation between ΔLFE and genomic % GC content was only seen in gene pairs where an RTS is less likely to occur (Pearson, n=128, r=0.546, p-value<10⁻¹⁰) (FIG. 14). Such correlation reflects stronger selection for RTS depletion in mid-operonic genes in organisms with higher % GC content. Considering that when % GC content is high, spontaneous mRNA secondary structures are more likely to appear, we expected and indeed observed, that more substantial purifying selection is required for RTS depletion.

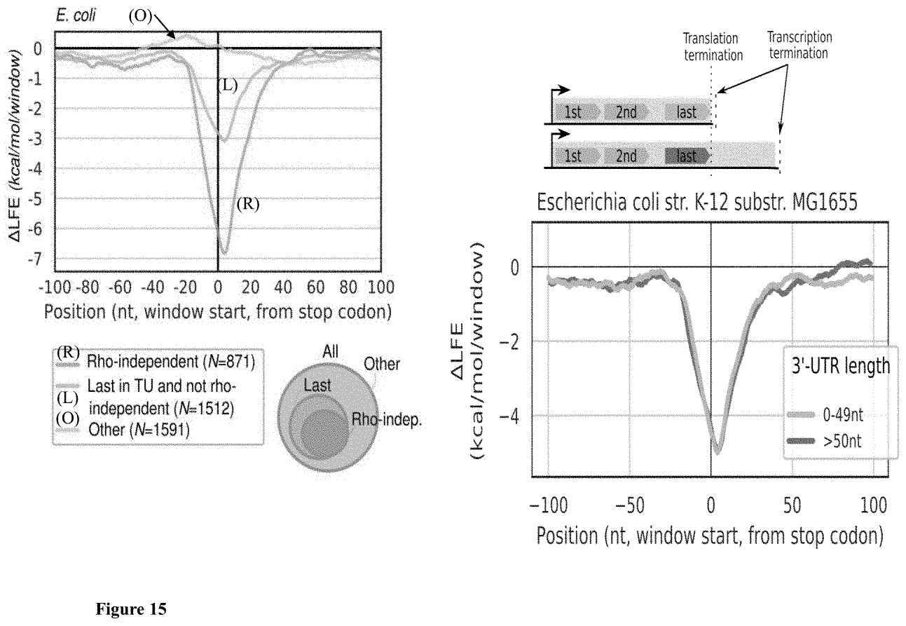

Lastly, there was explored whether RTS regions in the E. coli genome are enriched in any sequence motifs. Two uncharacterized motifs were identified but only in a small subset of genes, and as such, are unlikely to control re-initiation or account for RTS selection (FIG. 18). These results, together with the demonstrated lack of RTS linkage to transcription termination (FIGS. 6 and 15), are all consistent with the RTS playing a major role in bacterial translation re-initiation.

For each of the 128 bacterial species examined herein, all genes were separated into two groups following these conditions: Group 1) Genes with downstream intergenic distances of less than 25 nucleotides to the next CDS and are on the same strand. In this group, RTS is less expected, and enrichment of mid-operonic genes is expected. Group 2) Genes with a downstream intergenic distance of more than 25 nucleotides to the next CDS or are on opposite strands of the DNA. Three genomic traits where explored: a) % GC content, the proportion of GC in the genome (i.e., % GC); b) the proportion of genes in the genome, which are followed by a downstream gene on an opposite strand; this measure is used as a proxy to the length and number of operons in the species genome; and c) the average intergenic distance between all genes in a species genome. This measure is used as a proxy to the compression of the host genome, which is suspected of having implications regarding the usage, number, and size of operons.

The mean ΔLFE around the stop codons of all genes in each species was calculated, and the minimum ΔLFE found in the region between −10nt and 20nt relative to the first nucleotide of the 3′-UTR, was used as the ΔLFE value for each species.

With respect to a potential linkage to transcription termination, the fact that a stable mRNA structure down-stream of a stop codon could be functionally related to transcription termination since rho-independent transcription terminators can form stable mRNA hairpins was controlled for. Therefore, to distinguish the role of the RTS in regulating translation re-initiation from transcription termination, all 871 known or suspected genes that terminate with a rho-independent terminator sequence were removed from the analysis (FIG. 14, left). The RTS signal remained (Wilcoxon test, p-val<10⁻¹⁶). The reduction in the effect is probably due to the fact that rho-independent terminators affected the analysis by biasing the sequences ˜40-60 nt downstream of the stop codon to more stable structures, thus interfering with our analysis around the stop codon, as the window size used was 40 nt. To further demonstrate the absence of a link between the RTS and transcription termination, two subsets of terminal and monocistronic genes were analyzed according to their experimentally measured 3′ UTR lengths (FIG. 14, right), with one group presenting short 3′ UTRs (<50 nt) and the other possessing long 3′ UTRs (>50 nt). Were the RTS signal linked to transcription termination, one would expect to see the RTS signal closer to the stop codons in the former and further away from the stop codon in the latter. However, no change in the position or magnitude of the RTS was observed. These analyses, taken together, demonstrate that the RTS is not linked to transcription termination.

### Example 5

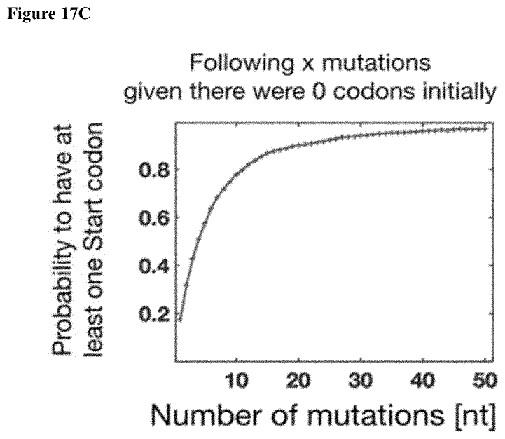

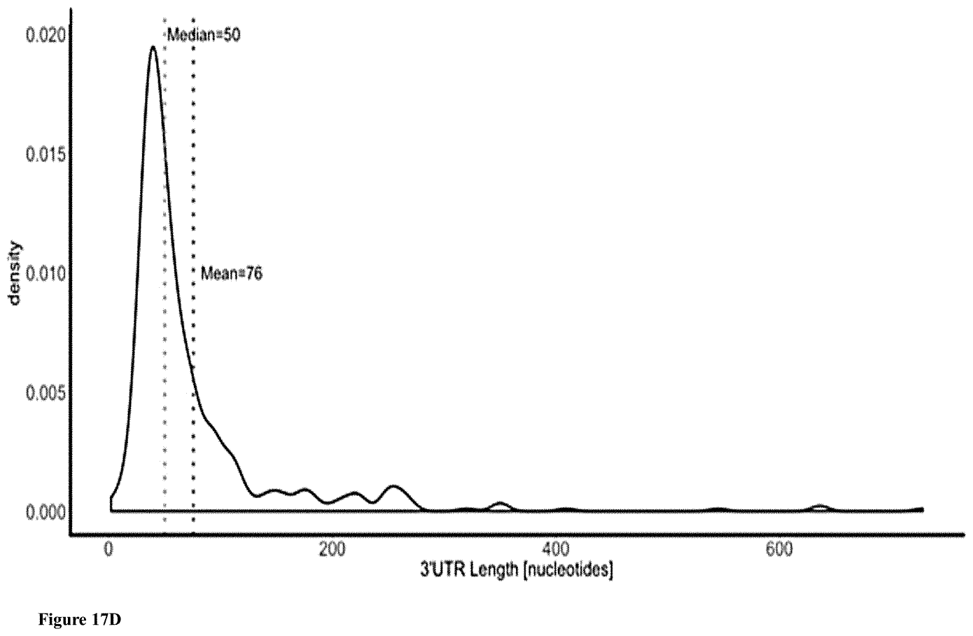

When considering the evolution of translation re-initiation, two solutions to avoid un-intended re-initiations when this is deleterious (for example, after the last gene of a polycistronic mRNA) are possible. The first involves depleting all efficient start codons. However, this is not optimal for three reasons: i) Even inefficient start codons could lead to basal expression by re-initiation; ii) ribosomes would wastefully spend time scanning for start codons which are depleted, resulting in a fitness cost; and iii) the probability of efficient start codons (one of the 6 most efficient) on a random 3′UTR sequence is >0.9 (FIG. 17A) if considering the median E. coli 3′ UTR length of 50 nucleotides (FIG. 17D). Moreover, the selection on the 3′ UTR would have to be extremely high to counter the ˜17% chance of an efficient start codon appearing after each single nucleotide mutation (FIG. 17B). This constraint is further compounded by consecutive mutations (FIG. 17C). To assess the length of E. coli 3′UTRs, we utilized RNA-seq data. The data revealed that in E. coli, the average 3′ UTR length is 76 nucleotides, with the median length being 50 nucleotides, a sufficient length to harbor significant mRNA secondary structure and require stringent selection to avoid start codon-generated mutations.

### Example 6

To test for the existence of conserved sequence motifs located near the stop codon, in the expected RTS region, which may account for the observed increase in folding energy, the MEME algorithm was used on the relevant sequences for putative RTS sequences and non-RTS sequences from all E. coli genes, all sequences are within the region of −10 to +60 bases around the stop codon of each gene (for annotation explanation see Materials and Methods). The search was limited to motifs with a length of 3-9nt and the number of motifs to 15 (top 10 results shown in FIG. 18).

The putative RTS regions contain two significantly enriched motifs. First, TTTTT was found in 359/2287 of the sequences (sites), which are the known Rho-independent terminator's uridine stretch. Second, ATAAAAAA, found in 148/2287 sequences. This motif is of unknown function. However, since it is present in a relatively small fraction of the genes, it was not further characterized.

The putative non-RTS regions also contain two significantly enriched motifs. First, GCTGGC was found in 95/1809 sequences. This motif is of unknown function. However, since it is present in a relatively small fraction of the genes, it was not further characterized. Second, ATGAA, found in 199/1809 sequences, represents a start-codon related enriched motif in downstream operon CDSs.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for producing a nucleic acid molecule optimized for expression of a second protein encoded by a second sequence comprising a translational start site (TSS) not more than 100 nucleotides away from a first stop codon of a first sequence encoding a first protein, the method comprising: introducing a mutation into a region from 7 to 75 nucleotides downstream of said first stop codon; wherein said mutation increases folding energy of said region or of RNA encoded by said region.

2. The method of claim 1, wherein said nucleic acid molecule is at least one of:
a. an RNA molecule;
b. a DNA molecule encoding a single RNA molecule comprising said first sequence encoding said first protein and said second sequence encoding said second protein;
c. devoid of an internal ribosome entry site (IRES) between said first sequence encoding said first protein and said second sequence encoding said second protein; and
d. a combination thereof.

3. (canceled)

4. The method of claim 1, wherein said first stop codon is upstream of said TSS of said sequence encoding said second protein.

5. (canceled)

6. The method of claim 1, wherein said mutation is within a sequence selected from SEQ ID NO: 44-53, and wherein said mutation produces a sequence that does not comprise any of SEQ ID NO: 44-53.

7. (canceled)

8. (canceled)

9. (canceled)

10. (canceled)

11. The method of claim 1, comprising introducing a mutation into a region from 7 to 40 nucleotides downstream of said stop codon.

12. The method of claim 1, wherein said nucleic acid molecule further comprises at least one regulatory region operatively linked to a first coding sequence encoding said first protein, wherein said at least one regulatory region is sufficient to drive expression of said first coding sequence or wherein said nucleic acid molecule is genomic DNA and said introducing a mutation comprises genome editing.

13. (canceled)

14. A nucleic acid molecule comprising:
a. at least two coding sequences, wherein a start codon of a second coding sequence is within 100 nucleotides of a stop codon of a first coding sequence; and
b. a region from 7 to 75 nucleotides downstream of said stop codon of said first coding sequence, wherein said region comprises:
i. a fragment of a naturally occurring 3′ UTR comprising a mutation that increases folding energy of said region or of RNA encoded by said region;
ii. at least a portion of said second coding sequence comprising at least one codon substituted to a different codon wherein said substitution increases folding energy of said region or of RNA encoded by said region; or
iii. an artificial sequence configured such that a folding energy of said region or RNA encoded by said region is above a predetermined threshold.

15. The nucleic acid molecule of claim 14, wherein said nucleic acid molecule is at least one of:
a. an RNA molecule;
b. a DNA molecule encoding a single RNA molecule comprising said at least two coding sequences;
c. devoid of an internal ribosome entry site (IRES) between said at least two coding sequences;
d. comprising said stop codon of said first coding sequence is upstream of a translational start site of said second coding sequence;
e. comprising said start codon of said second coding sequence is within 50 nucleotides of said stop codon of said first coding sequence; and
f. a combination thereof.

16. (canceled)

17. (canceled)

18. (canceled)

19. The nucleic acid molecule of claim 14, wherein said region comprises a sequence selected from GCTGGX12 (SEQ ID NO: 55) wherein X₁₂ is selected from C and T, ATTGAAX₁₃X₁₄ (SEQ ID NO: 56) wherein X₁₃ is A, T or C and X₁₄ is A or C, CTGX₁₅TGX₁₆ (SEQ ID NO: 57) wherein X₁₅ is A or C and X₁₆ is A, C or G, X₁₇GX₁₈X₁₉GCGX₂₀G (SEQ ID NO: 58) wherein X₁₇ is T or C, X₁₈ is T or C, X₁₉ is C or G, X₂₀ is T or C, X₂₁AX₂₂X₂₃AATX₂₄A (SEQ ID NO: 59) wherein X₂₁ is A or C, X₂₂ is A or G, X₂₃ is A or C, X₂₄ is A or G, TX₂₅GCCGC (SEQ ID NO: 60) wherein X₂₅ is C or T, X₂₆TGAAATX₂₇A (SEQ ID NO: 61) wherein X₂₆ is C or G and X₂₇ is G or A, GCCX₂₈GGC (SEQ ID NO: 62) wherein X₂₈ is T or G, TX₂₉TTTAX₃₀X₃₁G (SEQ ID NO: 63) wherein X₂₉ is T or C, X₃₀ is T or C, X₃₁ is T or C, ATGX₃₂X₃₃TX₃₄AX₃₅ (SEQ ID NO: 64) wherein X₃₂ is A, G or T, X₃₃ is G, C or T, X₃₄ is G or A and X₃₅ is A or T and X₃₆GCTGGX₁₂X₃₇X₃₈ (SEQ ID NO: 65), wherein X₃₆ is C, T or G, X₁₂ is C or T, X₃₇ is G, C or A and X₃₈ is C, T, G or A.

20. (canceled)

21. (canceled)

22. (canceled)

23. (canceled)

24. The nucleic acid molecule of claim 14, wherein said region is at least one of:
a. from 7 to 40 nucleotides downstream of said stop codon;
b. devoid of Rho-independent transcription terminators;
c. confirmed to induce ribosome translational re-initiation at said start codon of said second coding sequence;
d. configured to induce ribosome retention at said stop codon; and
e. a combination thereof.

25. The nucleic acid molecule of claim 14, wherein:
a. said fragment is a fragment of a naturally occurring bacterial 3′ UTR;
b. said fragment is between 20-100 nucleotides in length;
c. said substitution is a synonymous substitution; or
d. a combination thereof.

26. The nucleic acid molecule of claim 14, wherein:
a. said folding energy is local folding energy within a window of nucleotides;
b. said folding energy is local folding energy within a window of nucleotides and said increase or decrease is an increase or decrease of at least 1 kcal/mol/40 bp; or
c. said folding energy is local folding energy within a window of nucleotides and said predetermined threshold is −6 kcal/mol/40 bp.

27. (canceled)

28. (canceled)

29. (canceled)

30. (canceled)

31. An expression vector, comprising a nucleic acid molecule of claim 14.

32. An expression vector comprising:
a. a first region configured for insertion of a first coding sequence, or comprising a first coding sequence;
b. a second region configured for insertion of a second coding sequence, or comprising a second coding sequence, wherein a start of said second region is within 100 nucleotides from an end of said first region; and
c. a third region within 75 nucleotides downstream of said end of said first region, comprising:
i. a fragment of a naturally occurring 3′ UTR comprising a mutation that increases folding energy of said third region or RNA encoded by said third region; or
ii. an artificial sequence configured such that a folding energy of said third region or RNA encoded by said third region is above a predetermined threshold.

33. The vector of claim 32, wherein said vector is at least one of:
a. an RNA molecule;
b. a DNA molecule encoding a single RNA molecule comprising said first coding sequence and said second coding sequence;
c. devoid of an internal ribosome entry site (IRES) between said at least two coding sequences;
d. a bacterial expression vector; and
e. a combination thereof.

34. (canceled)

35. The vector of claim 32, wherein said first region comprises a first coding sequence and a stop codon of said second region is within 100 nucleotides of said stop codon or said second region comprises a second coding sequence and a translational start site (TSS) of said second coding sequence is within 100 nucleotides of said first region, said first region comprises a multiple cloning site (MCS), or both.

36. (canceled)

37. The vector of claim 32, wherein said third region comprises a sequence selected from SEQ ID NO: 55-65.

38. (canceled)

39. (canceled)

40. (canceled)

41. (canceled)

42. (canceled)

43. (canceled)

44. (canceled)

45. The vector of claim 32, wherein said fragment is
a. a fragment of a naturally occurring bacterial 3′ UTR;
b. is between 20-100 nucleotides in length, or
c. both.

46. The vector of claim 32, wherein said increase or decrease is an increase or decrease of at least 1 kcal/mol/40 bp, or wherein said predetermined threshold is −6 kcal/mol/40 bp.

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. A computer program product comprising a non-transitory computer-readable storage medium having program instructions embodied therewith, the program instructions executable by at least one hardware processor configured to perform a method of claim 1, comprising:
a. receive a sequence of a nucleic acid molecule comprising at least two coding sequences, wherein a start codon of a second coding sequence is proximal to a stop codon of a first coding sequence;
b. determine within a region around a stop codon of the first coding sequence at least one mutation that increases folding energy of the first region or RNA encoded by the first region; and
c. output
i. a mutated sequence of the nucleic acid molecule comprising the at least one mutation, or
ii. a list of possible mutations in the region that increase folding energy of the region or RNA encoded by the region.

59. (canceled)


SEQUENCE LISTING (66 sequences)

SEQ ID NO: 1 - DNA, 48 bp, synthetic construct
  misc_feature 1..48: note = Synthetic
  source 1..48: mol_type = other DNA
ataacaatttcacacagaaacagaagctggttctggcgaatagactag

SEQ ID NO: 2 - DNA, 28 bp, synthetic construct
  misc_feature 1..28: note = Synthetic
  source 1..28: mol_type = other DNA
ttctgtttctgtgtgaaattgttatccg

SEQ ID NO: 3 - DNA, 20 bp, synthetic construct
  misc_feature 1..20: note = Synthetic
  source 1..20: mol_type = other DNA
gacggtccggttatgcagaa

SEQ ID NO: 4 - DNA, 20 bp, synthetic construct
  misc_feature 1..20: note = Synthetic
  source 1..20: mol_type = other DNA
ttcagcgtcgtagtgaccac

SEQ ID NO: 5 - DNA, 20 bp, synthetic construct
  misc_feature 1..20: note = Synthetic
  source 1..20: mol_type = other DNA
caagctcccagtaccatggc

SEQ ID NO: 6 - DNA, 20 bp, synthetic construct
  misc_feature 1..20: note = Synthetic
  source 1..20: mol_type = other DNA
gcgctcttgtacatagccct

SEQ ID NO: 7 - DNA, 18 bp, synthetic construct
  misc_feature 1..18: note = Synthetic
  source 1..18: mol_type = other DNA
cggtgaatacgttcycgg

SEQ ID NO: 8 - DNA, 19 bp, synthetic construct
  misc_feature 1..19: note = Synthetic
  source 1..19: mol_type = other DNA
ggttaccttgttacgactt

SEQ ID NO: 9 - DNA, 69 bp, synthetic construct
  misc_feature 1..69: note = Synthetic
  source 1..69: mol_type = other DNA
tagactagtttacttccctcctctattctatcaaagggcgaggagctctttactggcgtagtaccaatt

SEQ ID NO: 10 - DNA, 69 bp, synthetic construct
  misc_feature 1..69: note = Synthetic
  source 1..69: mol_type = other DNA
tagactagtggcccgtcaacttgtgtggtttataagggcgaggagctctttactggcgtagtaccaatt

SEQ ID NO: 11 - DNA, 69 bp, synthetic construct
  misc_feature 1..69: note = Synthetic
  source 1..69: mol_type = other DNA
tagactagttgggagatgaatttaaaccggaacaagggcgaggagctctttactggcgtagtaccaatt

SEQ ID NO: 12 - DNA, 69 bp, synthetic construct
  misc_feature 1..69: note = Synthetic
  source 1..69: mol_type = other DNA
tagactagtccaacactggtgtttcgcggatggaagggcgaggagctctttactggcgtagtaccaatt

SEQ ID NO: 13 - DNA, 69 bp, synthetic construct
  misc_feature 1..69: note = Synthetic
  source 1..69: mol_type = other DNA
tagactagttcccctgaacctatattgcttgctaagggcgaggagctctttactggcgtagtaccaatt

SEQ ID NO: 14 - DNA, 69 bp, synthetic construct
  misc_feature 1..69: note = Synthetic
  source 1..69: mol_type = other DNA
tagactagtctaactgtacaactcttactgtcgaagggcgaggagctctttactggcgtagtaccaatt

SEQ ID NO: 15 - DNA, 69 bp, synthetic construct
  misc_feature 1..69: note = Synthetic
  source 1..69: mol_type = other DNA
tagactagtcaaattgttttggatcggaggaggaagggcgaggagctctttactggcgtagtaccaatt

SEQ ID NO: 16 - DNA, 69 bp, synthetic construct
  misc_feature 1..69: note = Synthetic
  source 1..69: mol_type = other DNA
tagactagtggttttagggcggatcaattgttaaagggcgaggagctctttactggcgtagtaccaatt

SEQ ID NO: 17 - DNA, 69 bp, synthetic construct
  misc_feature 1..69: note = Synthetic
  source 1..69: mol_type = other DNA
tagactagtcggggaaaaagggcggtgcgatgtaagggcgaggagctctttactggcgtagtaccaatt

SEQ ID NO: 18 - DNA, 69 bp, synthetic construct
  misc_feature 1..69: note = Synthetic
  source 1..69: mol_type = other DNA
tagactagtatccgtatattgttattggtcctgaagggcgaggagctctttactggcgtagtaccaatt

SEQ ID NO: 19 - DNA, 69 bp, synthetic construct
  misc_feature 1..69: note = Synthetic
  source 1..69: mol_type = other DNA
tagactagtggcgcgcctcttaatatgggtcgtaagggcgaggagctctttactggcgtagtaccaatt

SEQ ID NO: 20 - DNA, 69 bp, synthetic construct
  misc_feature 1..69: note = Synthetic
  source 1..69: mol_type = other DNA
tagactagtgcgtctattccgccgcccagccgtaagggcgaggagctctttactggcgtagtaccaatt

SEQ ID NO: 21 - DNA, 69 bp, synthetic construct
  misc_feature 1..69: note = Synthetic
  source 1..69: mol_type = other DNA
tagactagtccagtggcttcaagctcactgcctaagggcgaggagctctttactggcgtagtaccaatt

SEQ ID NO: 22 - DNA, 69 bp, synthetic construct
  misc_feature 1..69: note = Synthetic
  source 1..69: mol_type = other DNA
tagactagtgtatgtgaagccttggcgacgtataagggcgaggagctctttactggcgtagtaccaatt

SEQ ID NO: 23 - DNA, 69 bp, synthetic construct
  misc_feature 1..69: note = Synthetic
  source 1..69: mol_type = other DNA
tagactagtatgatttctacagtcaaaagggataagggcgaggagctctttactggcgtagtaccaatt

SEQ ID NO: 24 - DNA, 69 bp, synthetic construct
  misc_feature 1..69: note = Synthetic
  source 1..69: mol_type = other DNA
tagactagtggcagacactgtatgtatatattgaagggcsaggagctctttactggcgtaswaccaatt

SEQ ID NO: 25 - DNA, 69 bp, synthetic construct
  misc_feature 1..69: note = Synthetic
  source 1..69: mol_type = other DNA
tagactagtgaggcactgataatgtgtttggacaagggcgaggagctctttactggcgtagtaccaatt

SEQ ID NO: 26 - DNA, 69 bp, synthetic construct
  misc_feature 1..69: note = Synthetic
  source 1..69: mol_type = other DNA
tagactagtcgtaaacgaatgatgtcgtggcgtaagggcgaggagctctttactggcgtagtaccaatt

SEQ ID NO: 27 - DNA, 69 bp, synthetic construct
  misc_feature 1..69: note = Synthetic
  source 1..69: mol_type = other DNA
tagactagtatgttgtgttcaaacgaaatccagaagggcgaggagctctttactggcgtagtaccaatt

SEQ ID NO: 28 - DNA, 69 bp, synthetic construct
  misc_feature 1..69: note = Synthetic
  source 1..69: mol_type = other DNA
tagactagtaaaaaaatgtggcggcaaaatggaaagggcgaggagctctttactggcgtagtaccaatt

SEQ ID NO: 29 - DNA, 69 bp, synthetic construct
  misc_feature 1..69: note = Synthetic
  source 1..69: mol_type = other DNA
tagactagttgggtatcaatggcaatttctcttaagggcgaggagctctttactggcgtagtaccaatt

SEQ ID NO: 30 - DNA, 69 bp, synthetic construct
  misc_feature 1..69: note = Synthetic
  source 1..69: mol_type = other DNA
tagactagtatggctaggttaatggctggcaacaagggcgaggagctctttactggcgtagtaccaatt

SEQ ID NO: 31 - DNA, 69 bp, synthetic construct
  misc_feature 1..69: note = Synthetic
  source 1..69: mol_type = other DNA
tagactagtttgctttcgttcaatttaaactataagggcgaggagctctttactggcgtagtaccaatt

SEQ ID NO: 32 - DNA, 69 bp, synthetic construct
  misc_feature 1..69: note = Synthetic
  source 1..69: mol_type = other DNA
tagactagttggcccttgatttcacctatgttaaagggcgaggagctytttactggcgtagtaccaatt

SEQ ID NO: 33 - DNA, 69 bp, synthetic construct
  misc_feature 1..69: note = Synthetic
  source 1..69: mol_type = other DNA
tagactagtcggtcgattagttggatgtatgctaagggcgaggagctctttactggcgtagtaccaatt

SEQ ID NO: 34 - DNA, 69 bp, synthetic construct
  misc_feature 1..69: note = Synthetic
  source 1..69: mol_type = other DNA
tagactagtgtaaatttaatgagttctcgtgagaagggcgaggagctctttactggcgtagtaccaatt

SEQ ID NO: 35 - DNA, 69 bp, synthetic construct
  misc_feature 1..69: note = Synthetic
  source 1..69: mol_type = other DNA
tagactagttcagcacatttaggtgtgccgtacaagggcgaggagctctttactggcgtagtaccaatt

SEQ ID NO: 36 - DNA, 69 bp, synthetic construct
  misc_feature 1..69: note = Synthetic
  source 1..69: mol_type = other DNA
tagactagttctcacctggaaccgaataatgggaagggcgaggagctctttactggcgtagtaccaatt

SEQ ID NO: 37 - DNA, 69 bp, synthetic construct
  misc_feature 1..69: note = Synthetic
  source 1..69: mol_type = other DNA
tagactagtttgctttggtgtgcgaaggtcccgaagggcgaggagctctttactggcgtagtaccaatt

SEQ ID NO: 38 - DNA, 69 bp, synthetic construct
  misc_feature 1..69: note = Synthetic
  source 1..69: mol_type = other DNA
tagactagtcccgtgccatgtagaaagaatcagaagggcgaggagctctttactggcgtagtaccaatt

SEQ ID NO: 39 - DNA, 69 bp, synthetic construct
  misc_feature 1..69: note = Synthetic
  source 1..69: mol_type = other DNA
tagactagtaagatgaacctaaaaatgtctccaaagggcgaggagctctttactggcgtagtaccaatt

SEQ ID NO: 40 - DNA, 69 bp, synthetic construct
  misc_feature 1..69: note = Synthetic
  source 1..69: mol_type = other DNA
tagactagtgaggcactgcgaatgtgtttgaacaagggcgaggagctctttactggcgtagtaccaatt

SEQ ID NO: 41 - DNA, 69 bp, synthetic construct
  misc_feature 1..69: note = Synthetic
  source 1..69: mol_type = other DNA
tagactagtggcagacactgtatgtatatattgaagggcgaggagctctttactggcgtagtaccwatt

SEQ ID NO: 42 - DNA, 1479 bp, synthetic construct
  misc_feature 1..1479: note = Synthetic
  misc_feature 721..744: note = n is a, c, g, or t
  source 1..1479: mol_type = other DNA
atggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgctatgggatccgctggctccgctgctggttctggcgaatagactagtnnnnnnnnnnnnnnnnnnnnnnnnaagggcgaggagctctttactggcgtagtaccaattctcgtagagctcgatggcgatgtaaatggccataagttttccgtacgcggcgagggcgagggcgatgcaactaacggcaagctcactctcaagtttatttgtactactggcaagctcccagtaccatggccaactctcgtaactactctgacctatggcgtacaatgtttttcccgctatccagatcacatgaagcaacatgatttttttaagtccgcaatgccagagggctatgtacaagagcgcactattagctttaaggatgatggcacctataagactcgcgcagaggtaaagtttgagggcgatactctcgtaaatcgcattgagctcaagggcattgattttaaggaggatggcaatattctcggccataagctggagtataatttcaattcccataatgtatatattaccgcagataagcaaaagaatggcattaaggcgaattttaagattcgccataatgtggaggatggctccgtacaactcgcagatcattatcaacaaaatactccaattggcgatggcccagtactcctcccagataatcattatctctccactcaatccgtgctctccaaagatccaaatgagaagcgcgatcacatggtactcctggagtttgtaactgcagcaggcattactcatggcatggatgagctctataagctcgagcaccaccaccaccaccactaa

SEQ ID NO: 43 - DNA, 855 bp, synthetic construct
  misc_feature 1..855: note = Synthetic
  misc_feature 97..120: note = n is a, c, g, or t
  source 1..855: mol_type = other DNA
ttgctttgtgagcggataacaattataatagattcaattgtgagcggataacaatttcacacagaaacagaagctggttctggcgaatagactagtnnnnnnnnnnnnnnnnnnnnnnnnaagggcgaggagctctttactggcgtagtaccaattctcgtagagctcgatggcgatgtaaatggccataagttttccgtacgcggcgagggcgagggcgatgcaactaacggcaagctcactctcaagtttatttgtactactggcaagctcccagtaccatggccaactctcgtaactactctgacctatggcgtacaatgtttttcccgctatccagatcacatgaagcaacatgatttttttaagtccgcaatgccagagggctatgtacaagagcgcactattagctttaaggatgatggcacctataagactcgcgcagaggtaaagtttgagggcgatactctcgtaaatcgcattgagctcaagggcattgattttaaggaggatggcaatattctcggccataagctggagtataatttcaattcccataatgtatatattaccgcagataagcaaaagaatggcattaaggcgaattttaagattcgccataatgtggaggatggctccgtacaactcgcagatcattatcaacaaaatactccaattggcgatggcccagtactcctcccagataatcattatctctccactcaatccgtgctctccaaagatccaaatgagaagcgcgatcacatggtactcctggagtttgtaactgcagcaggcattactcatggcatggatgagctctataagctcgagcaccaccaccaccaccactaa

SEQ ID NO: 44-66 - skipped (23 entries)